(19) United States
(12) Patent Application Publication
(10) Pub. No.: US 2006/0276372 A1
(43) Pub. Date: Dec. 7, 2006

(54) Carotenoids, carotenoid analogs, or carotenoid derivatives for the treatment of proliferative disorders

(76) Inventors: Samuel F. Lockwood, Lago Vista, TX (US); Geoff Nadolski, Karlua, HI (US); Dean Allen Frey, Albany, NY (US); Mark McLaws, Ballston Lake, NY (US); Timothy J. King, Kanedhe, HI (US); David Burdick, Guilderland, NY (US)

Correspondence Address: MEYERTONS, HOOD, KIVLIN, KOWERT & GOETZEL, P.C., 700 LAVACA, SUITE 800, AUSTIN, TX, 78701, US

(21) Appl. No.: 11/372,353

(22) Filed: Mar. 9, 2006

Related U.S. Application Data
(60) Provisional application No. 60/659,983, filed on Mar. 9, 2005.

(51) Int. Cl. A61K 38/16; A61K 31/7034; A61K 31/7072; A61K 31/66; A61K 31/192; A61K 31/195; A61K 31/185; A61K 31/12; A61K 31/13

(52) U.S. Cl. 514/2; 514/25; 514/43; 514/690; 514/561; 514/553; 514/125; 514/666; 514/560

(57) ABSTRACT

A method and system used for treating proliferative disorders using carotenoids, carotenoid analogs, and/or carotenoid derivatives. The method and system may be used for chemoprevention and/or chemotherapy. The method and system may induce apoptosis in target cells, tissues, and/or organs. The analog, derivative, or intermediate may be administered to a cell, a group of cells, a tissue, an organ or a subject, such that at least a portion of the undesirable consequences of the proliferative disorder are thereby reduced.

### CROSS REFERENCE TO RELATED APPLICATION

This application claims the benefit of priority under 35 U.S.C. §119(e) to Provisional Patent Application Ser. No. 60/659,983, filed Mar. 9, 2005, entitled “CAROTENOIDS, CAROTENOID ANALOGS, OR CAROTENOID DERIVATIVES FOR THE INHIBITION OF NEOPLASTIC TRANSFORMATION.” The prior application is commonly assigned with the present invention, and the contents thereof are incorporated by reference in their entirety as though fully set forth herein.

### BACKGROUND OF THE INVENTION

1. Field of the Invention

The present invention generally relates to the fields of medicinal and synthetic chemistry. Specifically, the invention relates to the synthesis and use of water-soluble and water dispersible carotenoids, including analogs, derivatives, and intermediates thereof, for the treatment and inhibition of aberrant cell growth.

2. Description of the Relevant Art

Gap junctions are specialized regions of the cell membrane with clusters of hundreds to thousands of densely packed gap junction channels that directly connect the cytoplasmic compartment of two neighboring cells. The gap junction channels are composed of two hemichannels (connexons) provided by each of two neighboring cells. Each connexon consists of six proteins called connexins (Cx). The connexins are a large family of proteins all sharing the basic structure of four transmembrane domains, two extracellular loops, and a cytoplasmic loop. There is a high degree of conservation of the extracellular loops and transmembrane domains among species and connexin isoforms. The length of the C-terminus, however, varies considerably giving rise to the classification of the connexins on the basis of the molecular weight. The gap junction channel can switch between an open and a closed state by a twisting motion. In the open state ions and small molecules can pass through the pore. The conduction of the electrical impulse and intercellular diffusion of signaling molecules take place through the gap junctions and normally functioning gap junctions are therefore a prerequisite for normal intercellular communication. Normal intercellular communication is essential for for cellular homeostasis, proliferation and differentiation.

The link between abnormalities in connexins and disease has been established in humans as will appear in the sections below. One example is Chagas” disease caused by the protozoan parasite Trypanosoma cruzi. This disease is a major cause of cardiac dysfunction in Latin America. An altered Cx43 distribution has been observed in cells infected by Trypanosoma cruzi and this alteration may be involved in the genesis of the conduction disturbances characterizing the disease.

In a multicellular organism, co-ordination between cells is of paramount importance. Among the various means of cellular cross talk, gap junctions provide the most direct pathway. Gap junctions are one type of junctional complex formed between adjacent cells and consist of aggregated channels that directly link the interiors (cytoplasm) of neighbouring cells. In the adult mammal, gap junctions are found in most cell types with one known exception being circulating blood elements.

The pore diameter of the gap junction channel formed has been reported to be in the range of 0.8-1.4 nm. Gap junctions are relatively non-selective and allow the passage of molecules up to about 1000 Daltons (Da). Such substances are, i.a., ions, water, sugars, nucleotides, amino acids, fatty acids, small peptides, drugs, and carcinogens. Channel passage does not require ATP and appears to result from passive diffusion. This flux of materials between cells via gap junction channels is known as gap junctional intercellular communication (GJIC), which plays an important role in the regulation of cell metabolism, proliferation, and cell-to-cell signaling. One of the most significant physiological implications for GJIC is that gap junction coupled cells within a tissue are not individual, discrete entities, but are highly integrated with their neighbors, a “functional syncytium”. This property facilitates homeostasis and also permits the rapid, direct transfer of second messengers between cells to coordinate cellular responses within the tissue.

The process of GJIC is regulated by a variety of mechanisms that can be broadly divided into major categories. In one type of regulation the cellular quantity of gap junctions is controlled by influencing the expression, degradation, and cellular trafficking of connexins to the plasma membrane, or assembly of connexins into functional gap junctions. Impaired GJIC caused by the down-regulation of connexin expression, e.g. in tumor cells, is an example of this mode of regulation. Another type of regulation does not generally involve any gross alteration of the cellular levels of gap junctions or connexins, but induces opening or closure (gating) of existing gap junctions. Extracellular soluble factors, such as mitogens (e.g. DDT), hormones (e.g. catecholamines), anaesthetics (e.g. halothane), intracellular biomolecules (e.g. cAMP), and cell stress (e.g. mechanical or metabolic stress) can result in this type of regulation. Additionally, GJIC is regulated during the cell cycle and during cellular migration.

The mode of GJIC regulation or junctional gating has been widely studied for gap junctions especially gap junctions composed of Cx43. Some factors exert their inhibitory effects on GJIC indirectly, for example, by altering the lipid environment and cell membrane fluidity, whereas other GJIC inhibitors include oncogenes, growth factors, and tumor promoters, which induce various modifications of the Cx43. Disruption of junctional permeability may be necessary for mediating the specific biological functions of the latter group. These agents initiate complex signaling pathways consisting of the activation of kinases, phosphatases, and interacting proteins. Understanding the mechanisms of action of these GJIC modulators will not only define their respective signaling pathways responsible for junctional regulation, but will also provide experimental tools for characterising the biological functions of GJIC and connexins. Changes in the phosphorylation of specific sites of the cytoplasmic carboxy terminal domain of Cx43 appear to be pivotal to the opening and closing of the gap junctional channel. Phosphorylation of the carboxy terminal domain may also be important to the process of bringing Cx43 gap junctional hemicomplex to the cell membrane, its internalisation and degradation. Connexins have half-lives (hours) that are much shorter than most plasma membrane proteins (days), e.g. the half-life of Cx43 in rat heart is less than 1½ hours. Thus, regulation of the turnover rate would be an important factor in regulating GJIC.

The carboxy terminal domain contains putative phosphorylation sites for multiple protein kinases (PKA, PKC, PKG, MAPK, CaMkII and tyrosine kinase). Phosphorylation of these sites of the carboxy terminal domain results in closure of gap junctional channels and various inhibitors of Cx43 gap junctional channels use different signalling pathways to induce phosphorylation of the carboxy terminal domain. The cell type and the particular inhibitor determine which signalling pathways to be used and the type of the involved protein kinase points to the intracellular messenger system utilised. Thus activation of PKA requires involvement of the cAMP second messenger system while PKC requires involvement of the phosphoinositol intracellular signalling system.

Other mechanisms regulating channel gating include intracellular levels of hydrogen and calcium ions, transjunctional voltage, and free radicals. Decreased pH or pCa induce channel closure in a cell- and connexin-specific manner.

Many physiological roles besides growth control have been proposed for GJIC. Homeostasis: GJIC permits the rapid equilibration of nutrients, ions, and fluids between cells. This might be the most ancient, widespread, and important function for these channels. Electrical coupling: Gap junctions serve as electrical synapses in electrically excitable cells such as cardiac myocytes, smooth muscle cells, and neurons. In these tissues, electrical coupling permits more rapid cell-to-cell transmission of action potentials than chemical synapses. In cardiomyocytes and smooth muscle cells, this enables their synchronous contraction. Tissue response to hormones: GJIC may enhance the responsiveness of tissues to external stimuli. Second messengers such as cyclic nucleotides, calcium, and inositol phosphates are small enough to pass from hormonally activated cells to quiescent cells through junctional channels and activate the latter. Such an effect may increase the tissue response to an agonist. Regulation of embryonic development: Gap junctions may serve as intercellular pathways for chemical and/or electrical developmental signals in embryos and for defining the boundaries of developmental compartments. GJIC occurs in specific patterns in embryonic cells and the impairment of GJIC has been related to developmental anomalies and the teratogenic effects of many chemicals.

The intercellular communication ensures that the activities of the individual cells happen in a coordinated fashion and integrates these activities into the dynamics of a working tissue serving the organism in which it is set. It is therefore not very surprising that a wide variety of pathological conditions have been associated with decreased GJIC. The link between abnormalities in connexins and a range of disease states has been established both in vitro and in vivo. One example is regulation of gap junctional communication by a pro-inflammatory cytokine in airway epithelium, where Chanson et al. (Am J Pathol 2001 May;158(5):1775-84) found that decreased intercellular communication induced by TNF-α progressively led to inflammation.

In summary, mounting evidence linking malfunction, such as gating or closure or even absence, of gap junctions to an increased risk of disease has recently been collected. Few currently available drugs for the treatment of such diseases act as a facilitators of intercellular communication by facilitating or increasing gap junction function. Development of drugs that modulate Cx activity and/or functional GJIC would therefore improve methods of therapy and treatment of human disease.

GJIC in Cancer

Aberrant expression and function of several connexin proteins frequently occurs in cells exposed to tumor-promoting agents and during oncogenesis, both in cell culture systems and in tissues and tumors explanted from test animals and patients. Restoration of normal or near-normal levels of functional connexin proteins in neoplastic cells by transfecting the cells with connexin-encoding cDNAs exerts negative growth controls on neoplastic cells, suggesting that connexin proteins share important properties with known tumor-suppressor proteins. This hypothesis is supported by data establishing that GJIC is inhibited in cells or tumors exposed to tumor-promoting carcinogens or other oncogenic agents.

It is speculated that intact GJIC is a necessary, if not sufficient, biological function of metazoan cells for the regulation of growth control, differentiation and apoptosis of normal progenitor cells. Normal, contact-inhibited fibroblasts and epithelial cells have functional GJIC, while most, if not all, tumor cells have dysfunctional homologous or heterologous GJIC. Hallmark features of tumor cells include aberrant growth inhibitory mechanisms, prolonged or immortalized life-span, their lack of ability to reach a fully differentiated state, and their loss of ability to undergo apoptosis under normal conditions.

Chemical tumor promoters, growth factors and hormones have been shown to inhibit GJIC. Moreover, activation of certain cellular oncogenes, or the reduction in cellular levels of connexin proteins using anti-sense technology have been shown to reduce GJIC. Therefore, it is of therapeutic interest to identify compounds (anti-tumor and chemopreventive compounds) that restore, and/or prevent the loss of GJIC normally seen during neoplastic transformation.

Among the leading candidates for cancer chemoprevention are dietary carotenoids—pigments that in plants play a crucial role in protection from oxidative damage (Bertram et al., 1987). There is abundant epidemiological and laboratory evidence that carotenoids possess potent cancer chemopreventive properties in humans, independent of their antioxidant activity or their potential for conversion to retinoids. Unfortunately three major clinical trials of high-dose supplemental β-carotene, the carotenoid most frequently identified as protective against lung cancer, failed to demonstrate protection. In contrast, in two of these studies conducted in high-risk smokers and/or asbestos exposed workers, lung cancer incidence actually increased (Omenn et al., 1995; Albanes et al., 1996). The third study in largely non-smoking US physicians did not demonstrate protection or risk (Hennekens et al., 1996). In studies conducted in ferrets, one of the few laboratory models which absorb β-carotene to a comparable level as do humans, β-carotene was found to induce lung pathology and molecular changes consistent with retinoic acid deficiency as a consequence of enhanced catabolism of this important regulator of cell differentiation (Wang et al., 2003). These data suggest that use of carotenoids without potential for conversion to vitamin A may provide protection and avoid this toxicity. Recent studies using lycopene, a non-pro-vitamin A carotenoid, in the ferret model showed protection against tobacco-induced pathology, without toxicity (Liu et al., 2003).

Astaxanthin (AST), another non-pro-vitamin A carotenoid, is found predominantly as a dietary source in shrimp, lobster and salmon, and as such is not a major circulating carotenoid as are lycopene and β-carotene. In experimental animal studies astaxanthin has been shown to be capable of inhibiting chemically-induced oral and bladder carcinogenesis (Tanaka et al., 1994; Tanaka et al., 1995). Astaxanthin has also been shown to be effective at stimulating the immune system (Jyonuchi et al., 1995; Jyonuchi et al, 1996; Chew et al., 1999). Similar to other carotenoids, astaxanthin is a powerful lipid-phase antioxidant, and has been reported to suppress production of inflammatory cytokines (Lee at al, 2003). Based on this evidence, astaxanthin has significant cancer chemopreventive potential.

Delivery of highly lipophilic carotenoids such as astaxanthin to biological systems has met with formidable challenges. The most commonly employed method of delivery is in “beadlet” form, a micro-disbursed solution of carotenoids in vegetable oil in a water-soluble matrix. Unfortunately, only β-carotene, canthaxanthin and lycopene have been so formulated, and studies using beadlets in most laboratory animals has been confounded by poor absorption. Delivery of astaxanthin and other carotenoids in cell culture was made possible by using the solvent tetrahydrofuran (THF), although this solvent is unsuitable for animal and clinical use (Cooney et al, 1993).

The need for a water-soluble and/or water-dispersible delivery system for carotenoids has led to the development of a highly bioavailable, water-dispersible, disodium salt disuccinate ester of astaxanthin (dAST), disclosed in United States Patent Application No.: 2004-0162329, published on Aug. 19, 2004, which is incorporated by reference as though fully set forth herein. This compound formed a pseudo-solution in water at concentrations of up to 8 mg/ml (approximately 10 mM), and bioavailability of dAST in vitro was enhanced by the addition of ethanol as a co-solvent to maintain the compound in monomeric form. Oral administration of dAST to mice resulted in rapid absorption of the compound, its cleavage to free astaxanthin, and accumulation in certain target tissues. dAST was shown to be an effective scavenger of free radicals in the aqueous phase using an in vitro human polymorphonuclear leukocyte assay with complete inhibition of the induced superoxide anion at mM concentrations. This compound has also been shown to significantly protect against cardiac ischemia-reperfusion (I/R) injury, generally considered to result from oxidative stress, at doses up to 75 mg/kg in a rat model of experimental infarction, and at 50 mg/kg in canines. Administration of an aqueous ethanolic dAST formulation to carcinogen-promoted 10T1/2 cells, an in vitro model of carcinogenesis, resulted in increased expression of the connexin protein CX43 and GJIC.

Carotenoids are a group of natural pigments produced principally by plants, yeast, and microalgae. The family of related compounds now numbers greater than 700 described members, exclusive of Z and E isomers. Fifty (50) have been found in human sera or tissues. Humans and other animals cannot synthesize carotenoids de novo and must obtain them from their diet. All carotenoids share common chemical features, such as a polyisoprenoid structure, a long polyene chain forming the chromophore, and near symmetry around the central double bond. Tail-to-tail linkage of two C₂₀ geranyl-geranyl diphosphate molecules produces the parent C₄₀ carbon skeleton. Carotenoids without oxygenated functional groups are called “carotenes”, reflecting their hydrocarbon nature; oxygenated carotenes are known as “xanthophylls.” Cyclization at one or both ends of the molecule yields 7 identified end groups (illustrative structures shown in FIG. 1). Examples of uses of carotenoid derivatives and analogs are illustrated in U.S. patent application Ser. No. 10/793,671 filed on Mar. 4, 2004, entitled “CAROTENOID ETHER ANALOGS OR DERIVATIVES FOR THE INHIBITION AND AMELIORATION OF DISEASE” by Lockwood et al. published on Jan. 13, 2005, as Publication No. US-2005-0009758 and PCT International Application Number PCT/US2003/023706 filed on Jul. 29, 2003, entitled “STRUCTURAL CAROTENOID ANALOGS FOR THE INHIBITION AND AMELIORATION OF DISEASE” by Lockwood et al. (International Publication Number WO 2004/011423 A2, published on Feb. 5, 2004) both of which are incorporated by reference as though fully set forth herein.

5-Lipoxygenase in Cancer

Cancer of the prostate is the most commonly diagnosed malignancy among men in the United States and Europe, killing thousands every year. Metastatic prostate cancer responds initially to androgen withdrawal therapy, but hormone resistance frequently (and some reports state universally) develops. Chemotherapeutic agents currently available have little or no impact on the survival of the patients with hormone-refractory prostate cancer. For this reason, metastatic prostate cancer almost always has a fatal outcome. Although the incidence of the localized, latent form of prostate cancer is the same globally regardless of ethnic origin, there is significant variation in the occurrence of metastatic disease between Western countries and Eastern countries, suggesting involvement of environmental factors in metastatic progression. The underlying molecular mechanism involved in the progression phase of the disease is an active area of current research.

Epidemiological evidence suggests a link between the incidence of prostate cancer and dietary fat intake. It has been documented that arachidonic acid can directly stimulate in vitro growth of both hormone-responsive and -nonresponsive human prostate cancer cells, which suggests a causal link between dietary fat and prostate cancer progression.

Arachidonic acid can be metabolized to produce a host of proinflammatory substances, called eicosanoids, that act as potent autocrine and paracrine regulators of cell biology. These substances are known to modulate diverse physiologic and pathologic responses, including growth and invasiveness of tumor cells as well as suppression of immune surveillance. Release of arachidonic acid and formation of eicosanoids have also been implicated in the action of a number of cytokines, including epidermal growth factor, platelet derived growth factor, and bombesin. The specific eicosanoid responsible for mitogenesis varies with the cytokine and the cell lineage involved, and has included prostaglandin E2 (PGE₂) as well as several lipoxygenase products. In addition to their role in regulating mitogenesis, various eicosanoids can either trigger or block apoptosis. As with mitogenesis, the specific eicosanoid involved in triggering or blocking apoptosis is cell lineage-dependent. For example, synthesis of PGE₂ plays a central role in the apoptosis required for egg release during ovulation. In contrast, PGE₂ blocks activation-induced apoptosis in CD4⁺/CD8⁺ T lymphocytes. In addition, both FAS and TNF receptor activation are associated with arachidonic acid release and eicosanoid formation in certain cell lineages. Recent evidence indicates that arachidonic acid suppresses ceramide-induced cell death in prostate cancer cells and that this suppression depends on formation of lipoxygenase products. It has previously been shown that arachidonic acid stimulates mitogenesis of human prostate cancer cells in vitro. This mitogenesis is blocked if further metabolism of arachidonic acid through 5-lipoxygenase is interrupted. Moreover, MK886, a specific inhibitor of 5-lipoxygenase, not only blocked the growth stimulation by arachidonic acid but, at a concentration of 10 mM, killed more than 90% of the cells in culture.

Ghosh et al. (Proc. Natl. Acad. Sci. USA, Vol. 95, pp. 13182-13187, October 1998) have reported that that cultured human prostate cancer cell lines constitutively produce 5-HETE (5-hydroxyeicosatetraenoic acid), a product of arachidonate 5-lipoxygenase activity, with no external stimulus, and that addition of exogenous arachidonic acid to the cells dramatically increases 5-HETE production. It was found that inhibiting the activity of the 5-lipoxygenase enzyme in these cells blocks production of 5-HETE and induces apoptosis in both hormone-responsive and hormone-nonresponsive human prostate cancer cells. The induction of apoptosis in these cells could be prevented by the simultaneous addition of the 5-HETE series of arachidonic acid metabolites, indicating a critical role of these metabolites in the survival of human prostate cancer cells. 5-HETE and its closely related, more potent inflammatory eicosanoid 5-oxo-EET can be considered “survival factors” for human prostate cancer cells.

Antioxidant Properties of Carotenoids

Free radicals are highly reactive molecules having one or more unpaired electrons in their outer orbital. Free radicals are involved in normal metabolism, and are always present in the human body, but normally at very low concentrations. There is considerable interest in understanding free radical biochemistry, since changes in the bioavailability of these molecules are believed to be involved in the early stages and progression of several diseases, such as cancer, inflammatory disease and cardiovascular, among others.

Carotenoids are a group of natural pigments produced principally by plants, yeast, and microalgae. The family of related compounds now numbers greater than 750 described members, exclusive of Z and E isomers. Humans and other animals cannot synthesize carotenoids de novo and must obtain them from their diet. All carotenoids share common chemical features, such as a polyisoprenoid structure, a long polyene chain forming the chromophore, and near symmetry around the central double bond. Tail-to-tail linkage of two C₂₀ geranyl-geranyl diphosphate molecules produces the parent C₄₀ carbon skeleton. Carotenoids without oxygenated functional groups are called “carotenes”, reflecting their hydrocarbon nature; oxygenated carotenes are known as “xanthophylls.” “Parent” carotenoids may generally refer to those natural compounds utilized as starting scaffold for structural carotenoid analog synthesis. Carotenoid derivatives may be derived from a naturally occurring carotenoid. Naturally occurring carotenoids may include lycopene, lycophyll, lycoxanthin, astaxanthin, beta-carotene, lutein, zeaxanthin, and/or canthaxanthin to name a few.

Cyclization at one or both ends of the molecule yields 7 identified end groups (illustrative structures shown in FIG. 1). Examples of uses of carotenoid derivatives and analogs are illustrated in U.S. patent application Ser. No. 10/793,671 filed on Mar. 4, 2004, entitled “CAROTENOID ETHER ANALOGS OR DERIVATIVES FOR THE INHIBITION AND AMELIORATION OF DISEASE” by Lockwood et al. published on Jan. 13, 2005, as Publication No. US-2005-0009758 and PCT International Application Number PCT/US2003/023706 filed on Jul. 29,2003, entitled “STRUCTURAL CAROTENOID ANALOGS FOR THE INHIBITION AND AMELIORATION OF DISEASE” by Lockwood et al. (International Publication Number WO 2004/011423 A2, published on Feb. 5, 2004) both of which are incorporated by reference as though fully set forth herein.

Documented carotenoid functions in nature include light-harvesting, photoprotection, and protective and sex-related coloration in microscopic organisms, mammals, and birds, respectively. A relatively recent observation has been the protective role of carotenoids against age-related diseases in humans as part of a complex antioxidant network within cells. This role is dictated by the close relationship between the physicochemical properties of individual carotenoids and their in vivo functions in organisms. The long system of alternating double and single bonds in the central part of the molecule (delocalizing the n-orbital electrons over the entire length of the polyene chain) confers the distinctive molecular shape, chemical reactivity, and light-absorbing properties of carotenoids. Additionally, isomerism around C═C double bonds yields distinctly different molecular structures that may be isolated as separate compounds (known as Z (“cis”) and E (“trans”), or geometric, isomers). Of the more than 750 described carotenoids, an even greater number of the theoretically possible mono-Z and poly-Z isomers are sometimes encountered in nature. The presence of a Z double bond creates greater steric hindrance between nearby hydrogen atoms and/or methyl groups, so that Z isomers are generally less stable thermodynamically, and more chemically reactive, than the corresponding all-E form. The all-E configuration is an extended, linear, and rigid molecule. Z-isomers are, by contrast, not simple, linear molecules (the so-called “bent-chain” isomers). The presence of any Z in the polyene chain creates a bent-chain molecule. The tendency of Z-isomers to crystallize or aggregate is much less than the all-E isomers. Additionally, Z isomers are more readily solubilized, absorbed, and transported in vivo than their all-E counterparts. This has important implications for enteral (e.g., oral) and parenteral (e.g., intravenous, intra-arterial, intramuscular, and subcutaneous) dosing in mammals.

Carotenoids with chiral centers may exist either as the R (rectus) or S (sinister) configurations. As an example, astaxanthin (with 2 chiral centers at the 3 and 3′ carbons) may exist as 4 possible stereoisomers: 3S, 3′S; 3R, 3′S and 3S, 3′R (identical meso forms); or 3R, 3′R. The relative proportions of each of the stereoisomers may vary by natural source. For example, Haematococcus pluvialis microalgal meal is 99% 3S, 3′S astaxanthin, and is likely the predominant human evolutionary source of astaxanthin. Krill (3R,3′R) and yeast sources yield different stereoisomer compositions than the microalgal source. Synthetic astaxanthin, produced by large manufacturers such as Hoffmann-LaRoche AG, Buckton Scott (USA), or BASF AG, are provided as defined geometric isomer mixtures of a 1:2:1 stereoisomer mixture [3S, 3′S; 3R, 3′S, 3′R,3S (meso); 3R, 3′R] of non-esterified, free astaxanthin. Natural source astaxanthin from salmon fish is predominantly a single stereoisomer (3S,3′S), but does contain a mixture of geometric isomers. Astaxanthin from the natural source Haematococcus pluvialis may contain nearly 50% Z isomers. As stated above, the Z conformational change may lead to a higher steric interference between the two parts of the carotenoid molecule, rendering it less stable, more reactive, and more susceptible to reactivity at low oxygen tensions. In such a situation, in relation to the all-E form, the Z forms: (1) may be degraded first; (2) may better suppress the attack of cells by reactive oxygen species such as superoxide anion; and (3) may preferentially slow the formation of radicals. Overall, the Z forms may initially be thermodynamically favored to protect the lipophilic portions of the cell and the cell membrane from destruction. It is important to note, however, that the all-E form of astaxanthin, unlike β-carotene, retains significant oral bioavailability as well as antioxidant capacity in the form of its dihydroxy- and diketo-substitutions on the β-ionone rings, and has been demonstrated to have increased efficacy over β-carotene in most studies. The all-E form of astaxanthin has also been postulated to have the most membrane-stabilizing effect on cells in vivo. Therefore, it is likely that the all-E form of astaxanthin in natural and synthetic mixtures of stereoisomers is also extremely important in antioxidant mechanisms, and may be the form most suitable for particular pharmaceutical preparations.

The antioxidant mechanism(s) of carotenoids, and in particular astaxanthin, includes singlet oxygen quenching, direct radical scavenging, and lipid peroxidation chain-breaking. The polyene chain of the carotenoid absorbs the excited energy of singlet oxygen, effectively stabilizing the energy transfer by delocalization along the chain, and dissipates the energy to the local environment as heat. Transfer of energy from triplet-state chlorophyll (in plants) or other porphyrins and proto-porphyrins (in mammals) to carotenoids occurs much more readily than the alternative energy transfer to oxygen to form the highly reactive and destructive singlet oxygen (¹O₂). Carotenoids may also accept the excitation energy from singlet oxygen if any should be formed in situ, and again dissipate the energy as heat to the local environment. This singlet oxygen quenching ability has significant implications in cardiac ischemia, macular degeneration, porphyria, and other disease states in which production of singlet oxygen has damaging effects. In the physical quenching mechanism, the carotenoid molecule may be regenerated (most frequently), or be lost. Carotenoids are also excellent chain-breaking antioxidants, a mechanism important in inhibiting the peroxidation of lipids. Astaxanthin can donate a hydrogen (H) to the unstable polyunsaturated fatty acid (PUFA) radical, stopping the chain reaction. Peroxyl radicals may also, by addition to the polyene chain of carotenoids, be the proximate cause for lipid peroxide chain termination. The appropriate dose of astaxanthin has been shown to completely suppress the peroxyl radical chain reaction in liposome systems. Astaxanthin shares with vitamin E this dual antioxidant defense system of singlet oxygen quenching and direct radical scavenging, and in most instances (and particularly at low oxygen tension in vivo) is superior to vitamin E as a radical scavenger and physical quencher of singlet oxygen.

Carotenoids, and in particular astaxanthin, are potent direct radical scavengers and singlet oxygen quenchers and possess all the desirable qualities of such therapeutic agents for inhibition or amelioration of ischemia-reperfusion (I/R) injury. Synthesis of novel carotenoid derivatives with “soft-drug” properties (i.e. activity in the derivatized form), with physiologically relevant, cleavable linkages to pro-moieties, can generate significant levels of free carotenoids in both plasma and solid organs. This is critically important, for in mammals, diesters of carotenoids generate the non-esterified or “free” parent carotenoid, and may be viewed as elegant synthetic and novel delivery vehicles with improved properties for delivery of free carotenoid to the systemic circulation and ultimately to target tissue. In the case of non-esterified, free astaxanthin, this is a particularly useful embodiment (characteristics specific to non-esterified, free astaxanthin below):
Lipid soluble in natural form; may be modified to become more water soluble;
Molecular weight of 597 Daltons [size <600 Daltons (Da) readily crosses the blood brain barrier, or BBB];
Long polyene chain characteristic of carotenoids effective in singlet oxygen quenching and lipid peroxidation chain breaking;
No pro-vitamin A activity in mammals (eliminating concerns of hypervitaminosis A and retinoid toxicity in humans).

The administration of antioxidants, which are potent singlet oxygen quenchers and direct radical scavengers, particularly of superoxide anion, should limit hepatic fibrosis and the progression to cirrhosis by affecting the activation of hepatic stellate cells early in the fibrogenetic pathway. Reduction in the level of ROS by the administration of a potent antioxidant can therefore be crucial in the prevention of the activation of both HSC and Kupffer cells. This protective antioxidant effect appears to be spread across the range of potential therapeutic antioxidants, including water-soluble (e.g., vitamin C, glutathione, resveratrol) and lipophilic (e.g., vitamin E, β-carotene, astaxanthin) agents. Therefore, a co-antioxidant derivative strategy in which water-soluble and lipophilic agents are combined synthetically is a particularly useful embodiment.

Vitamin E is generally considered the reference antioxidant. When compared with vitamin E, carotenoids are more efficient in quenching singlet oxygen in homogeneous organic solvents and in liposome systems. They are better chain-breaking antioxidants as well in liposomal systems. They have demonstrated increased efficacy and potency in vivo. They are particularly effective at low oxygen tension, and in low concentration, making them extremely effective agents in disease conditions in which ischemia is an important part of the tissue injury and pathology. These carotenoids also have a natural tropism for the liver after oral administration. Therefore, therapeutic administration of carotenoids should provide a greater benefit in limiting fibrosis than vitamin E.

Problems related to the use of some carotenoids and structural carotenoid analogs include: (1) the complex isomeric mixtures, including non-carotenoid contaminants, provided in natural and synthetic sources leading to costly increases in safety and efficacy tests required by such agencies as the FDA; (2) limited bioavailability upon administration to a subject; and (3) the differential induction of cytochrome P450 enzymes (this family of enzymes exhibits species-specific differences which must be taken into account when extrapolating animal work to human studies).

New methods, systems and compounds capable of modulating intracellular levels of connexin proteins, gap junctions and GJIC in cells that have undergone, or that are at risk of undergoing, neoplastic transformation would be useful therapeutic agents. Carotenoid analogs or derivatives displaying properties of increased water-dispersibility and bioavailability would be beneficial for such applications.

### SUMMARY

Methods for preventing or treating diseases resulting from impaired intercellular communication or impaired gap junction function are provided for herein. Illustrative diseases include those effecting the respiratory, circulatory or nervous systems, vision and hearing, dental tissues, smooth musculature, and transplantation of cells and tissues. Such methods can be used alone as the sole therapeutic regimen or in combination with one or more other established protocols for addressing a particular disease or condition. Carotenoid analogs or derivatives useful in the treatment methods contemplated herein are characterised in functioning as facilitators of GJIC.

More specifically the presently disclosed treatment methods relate to preventing or treating proliferative disorders caused, at least in part, by impaired gap junction function by facilitating (maintaining and/or restoring) the intercellular communication in the diseased cells and tissues occurring through gap junctions, preferably by administering a therapeutically effective amount of at least one carotenoid analog or derivative which facilitates CX43 expression and gap junction intercellular communication to a patient suffering from said disease.

In some embodiments, methods of reducing neoplastic transformation of a cell, a group of cells or in a subject may include administering to the cell, group of cells or subject an effective amount of a pharmaceutically acceptable formulation including a synthetic analog or derivative of a carotenoid.

In some embodiments, methods of modulating the amount of connexin 43 protein in a cell or in a group of cells may include administering to the cell, group of cells or to a subject, an effective amount of a pharmaceutically acceptable formulation including a synthetic analog or derivative of a carotenoid.

In some embodiments, methods of modulating the number of gap junctional complexes at or near the cell membrane of a cell or a group of cells may include administering to the cell, group of cells or to a subject, an effective amount of a pharmaceutically acceptable formulation including a synthetic analog or derivative of a carotenoid.

In some embodiments, methods of modulating GJIC between adjacent or substantially adjacent cells may include administering to the cell, group of cells or to a subject, an effective amount of a pharmaceutically acceptable formulation including a synthetic analog or derivative of a carotenoid.

In a further set of embodiments, methods for the treatment or prevention of proliferative diseases, in particular cancers that are dependent on the presence of one or more metabolites of the fatty acid arachidonate such as products of the enzyme 5-Lipoxygenase (5-LO), are provided for herein.

In an embodiment, contacting a neoplastic cell, such as a prostate cancer cell, with a pharmaceutically acceptable formulation containing an effective amount of synthetic analog or derivative of a carotenoid may inhibit or reduce the activity of 5-Lipoxygenase in the neoplastic cell. Inhibition or reduction of 5-Lipoxygenase in the neoplastic cell may, in certain embodiments, result in that cell undergoing apoptosis.

In an embodiment, prostate cancer cells may be induced to undergo apoptosis by contacting the cells with a pharmaceutically acceptable formulation containing an effective amount of a synthetic analog or derivative of a carotenoid. In an embodiment, the prostate cancer cells may be part of a solid prostate tumor present in a human subject.

The presently embodied treatment methods, including the administration of pharmaceutically acceptable formulations containing synthetic carotenoid analogs or derivatives, may be provided alone as a primary therapy, or may be provided in conjunction with one more more additional therapeutic agents (e.g. androgen withdrawal therapy) and/or radiation therapy as part of a therapeutic regimen. Such determination may be made by an appropriate healthcare provider and practitioner of ordinary skill in the art.

Administration of analogs or derivatives of carotenoids according to the preceding embodiments may at least partially inhibit and/or influence the occurrence or the progression of proliferative disorders. Proliferative disorders that may be influenced by administration of analogs or derivatives of carotenoids according to some embodiments may include those disorders that are characterized by aberrant or otherwise dysregulated cell growth, such as, for example, benign or malignant neoplasms or any other disorder characterized by the proliferation of anaplastic cells, and/or invasion of such cells into surrounding tissues or distal sites. Non-limiting examples of proliferative disorders that may be influenced according to some embodiments include neoplasia, such as, for example, brain cancer, bone cancer, epithelial cell-derived neoplasia (epithelial carcinoma), such as, for example, basal cell carcinoma, adenocarcinoma, gastrointestinal cancer, such as, for example, lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamus cell and basal cell cancers, prostate cancer, renal cell carcinoma, and other known cancers that effect epithelial cells throughout the body, benign and cancerous tumors, growths, polyps, adenomatous polyps, including, but not limited to, familial adenomatous polyposis.

In some embodiments, the administration of structural analogs or derivatives of carotenoids by one skilled in the art—including consideration of the pharmacokinetics and pharmacodynamics of therapeutic drug delivery—is expected to inhibit and/or ameliorate disease conditions associated with abnormal cell division. In some of the foregoing embodiments, analogs or derivatives of carotenoids administered to cells may be at least partially water-soluble.

“Water-soluble” structural carotenoid analogs or derivatives are those analogs or derivatives that may be formulated in aqueous solution, either alone or with one or more excipients. Water-soluble carotenoid analogs or derivatives may include those compounds and synthetic derivatives which form molecular self-assemblies, and may be more properly termed “water dispersible” carotenoid analogs or derivatives. Water-soluble and/or “water-dispersible” carotenoid analogs or derivatives may be preferred in some embodiments.

Water-soluble carotenoid analogs or derivatives may have a water solubility of greater than about 1 mg/mL in some embodiments. In certain embodiments, water-soluble carotenoid analogs or derivatives may have a water solubility of greater than about 10 mg/mL. In certain embodiments, water-soluble carotenoid analogs or derivatives may have a water solubility of greater than about 20 mg/mL. In certain embodiments, water-soluble carotenoid analogs or derivatives may have a water solubility of greater than about 25 mg/mL. In some embodiments, water-soluble carotenoid analogs or derivatives may have a water solubility of greater than about 50 mg/mL.

In some embodiments, water-soluble analogs or derivatives of carotenoids may be administered to a cell, a group of cells or to a subject alone or in combination with additional carotenoid analogs or derivatives.

In some embodiments, a method of treating a proliferative disorder may include administering to the subject an effective amount of a pharmaceutically acceptable formulation including a synthetic analog or derivative of a carotenoid. The synthetic analog or derivative of the carotenoid may have the structure

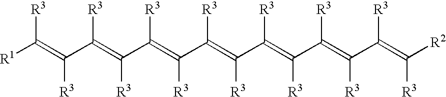

where each R³ is independently hydrogen or methyl, and where each R¹ and R² are independently: 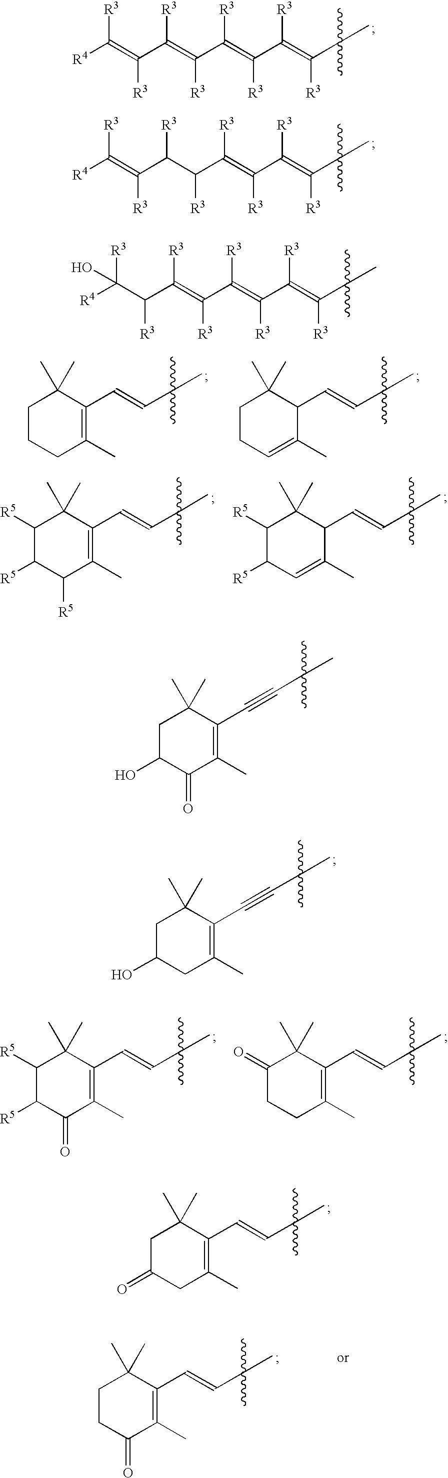 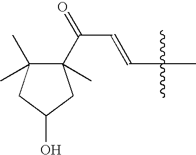
where R⁴ is hydrogen, methyl, or —CH₂OH; and where each R⁵is independently hydrogen or —OH.

In some embodiments, a method of inhibiting or reducing at least some of the side effects associated with therapeutic administration of COX-2 selective inhibitors may include administering to the subject an effective amount of a pharmaceutically acceptable formulation including a synthetic analog or derivative of a carotenoid. The synthetic analog or derivative of the carotenoid may have the structure

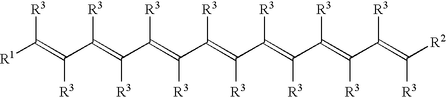

where each R³ is independently hydrogen or methyl, and where each R¹ and R² are independently: 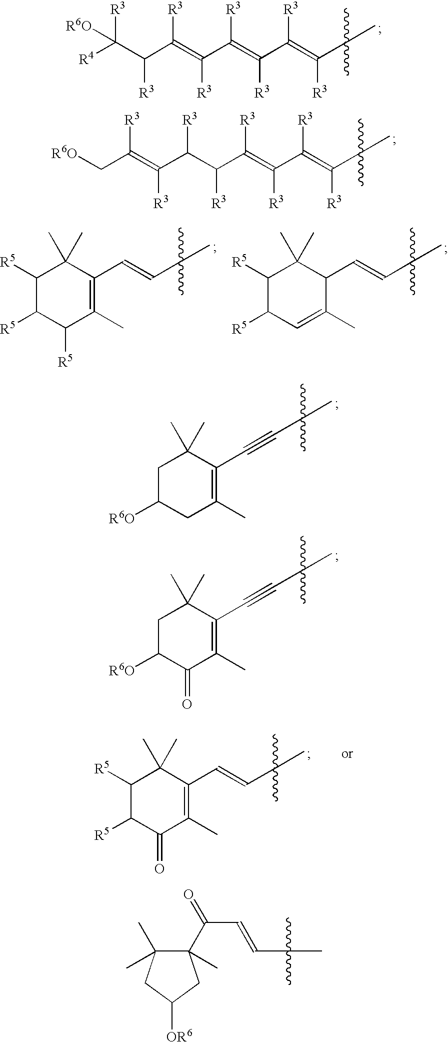
where R⁴ is hydrogen or methyl; where each R⁵ is independently hydrogen, —OH, or —OR⁶ wherein at least one R⁵ group is —OR⁶; wherein each R⁶ is independently: alkyl; aryl; -alkyl-N(R⁷)₂; -aryl-N(R⁷)₂; -alkyl-CO₂H; -aryl-CO₂H; —O—C(O)—R⁸;—P(O)(OR⁸)₂; —S(O)(OR⁸)₂; an amino acid; a peptide, a carbohydrate; —C(O)—(CH₂)ₙ—CO₂R⁹; —C(O)—OR⁹; a nucleoside residue, or a co-antioxidant; where R⁷ is hydrogen, alkyl, or aryl; wherein R⁸ is hydrogen, alkyl, aryl, benzyl, or a co-antioxidant; and where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9. Pharmaceutically acceptable salts of any of the above listed carotenoid derivatives may also be used to ameliorate at least some of the side effects associated with therapeutic administration of COX-2 selective inhibitors Each co-antioxidant may be independently Vitamin C, Vitamin C analogs, Vitamin C derivatives, Vitamin E, Vitamin E analogs, Vitamin E derivatives, flavonoids, flavonoid derivatives, or flavonoid analogs. Flavonoids include, but are not limited to, quercetin, xanthohumol, isoxanthohumol, or genistein. Selection of the co-antioxidant should not be seen as limiting for the therapeutic application of the current invention.

In some embodiments, a pharmaceutical composition is provided that may include one or more synthetic carotenoids (“a co-formulation” strategy), or synthetic derivatives or analogs thereof, in combination with one or more selective 5-LO inhibitor drugs, chemotherapeutic agents and/or in conjunction with radiation tharapy. Certain embodiments may further directed to pharmaceutical compositions that include combinations of two or more carotenoids or synthetic analogs or derivatives thereof. In an embodiment, a pharmaceutical composition may include chiral astaxanthin in combination with a 5-LO inhibitor drug or chemotherapeutic agent. In an embodiment, a pharmaceutical composition may include a synthetic derivative of lycophyll in combination with a 5-LO inhibitor drug or chemotherapeutic agent. The pharmaceutical compositions may be adapted to be administered orally, or by one or more parenteral routes of administration. In an embodiment, the pharmaceutical composition may be adapted such that at least a portion of the dosage of carotenoid or synthetic derivative or analog thereof is delivered prior to, during, or after at least a portion of the 5-LO inhibitor drugs, chemotherapeutic agents and/or radiation therapy is delivered.

In some embodiments, separate pharmaceutical compositions are provided, such that the 5-LO inhibitor drugs or additional chemotherapeutic agents are delivered separately from carotenoid, or synthetic derivatives or analogs thereof (sometimes referred to in the art as a “co-administration” strategy). The pharmaceutical compositions may be adapted to be administered orally, or by one or more parenteral routes of administration. In an embodiment, the pharmaceutical composition may be adapted such that at least a portion of the dosage of the carotenoid or synthetic derivative or analog thereof is delivered prior to, during, or after at least a portion of the 5-LO inhibitor drugs or additional chemotherapeutic agents are administered to the subject. The carotenoid, carotenoid analogs and/or derivatives may also be administered alone.

Embodiments directed to pharmaceutical compositions may further include appropriate vehicles for delivery of said pharmaceutical composition to a desired site of action (i.e., the site a subject's body where the biological effect of the pharmaceutical composition is most desired). Pharmaceutical compositions including xanthophyll carotenoids or analogs or derivatives of astaxanthin, lutein or zeaxanthin that may be administered orally or intravenously may be particularly advantageous for and suited to embodiments described herein. In yet a further embodiment, an injectable astaxanthin formulation or a structural analog or derivative may be administered with a astaxanthin, zeaxanthin or lutein structural analog or derivative and/or other carotenoid structural analogs or derivatives, or in formulation with antioxidants and/or excipients that further the intended purpose. In some embodiments, one or more of the xanthophyll carotenoids or synthetic analogs or derivatives thereof may be at least partially water-soluble.

Certain embodiments may further directed to pharmaceutical compositions including combinations two or more structural carotenoid analogs or derivatives. Pharmaceutical compositions including injectable structural carotenoid analogs or derivatives of lutein may be particularly advantageous for the methods described herein. In yet a further embodiment, an injectable lutein structural analog or derivative may be administered with a zeaxanthin structural analog or derivative and/or other carotenoid structural analogs or derivatives, or in formulation with antioxidants and/or excipients that further the intended purpose. In some embodiments, one or more of the lutein structural analogs or derivatives are water-soluble.

### BRIEF DESCRIPTION OF THE DRAWINGS

The above brief description as well as further objects, features and advantages of the methods and apparatus of the present invention will be more fully appreciated by reference to the following detailed description of presently preferred but nonetheless illustrative embodiments in accordance with the present invention when taken in conjunction with the accompanying drawings.

FIG. 1 depicts a graphic representation of several examples of the structures of several xanthophyll carotenoids and synthetic derivatives or analogs that may be used according to some embodiments. (A) astaxanthin; (B) lutein; (C) zeaxanthin; (D) disuccinic acid astaxanthin ester; (E) disodium disuccinic acid ester astaxanthin salt (Cardax™); and (F) divitamin C disuccinate astaxanthin ester; (G) tetrasodium diphosphate astaxanthin ester;

FIG. 2 depicts a time series of the UV/V is absorption spectra of the disodium disuccinate derivative of natural source lutein in water;

FIG. 3 depicts a UV/V is absorption spectra of the disodium disuccinate derivative of natural source lutein in water (λₘₐₓ=443 nm), ethanol (λₘₐₓ=446 nm), and DMSO (λₘₐₓ=461 nm);

FIG. 4 depicts a UV/V is absorption spectra of the disodium disuccinate derivative of natural source lutein in water (λₘₐₓ=442 nm) with increasing concentrations of ethanol;

FIG. 5 depicts a time series of the UV/V is absorption spectra of the disodium diphosphate derivative of natural source lutein in water;

FIG. 6 depicts a UV/V is absorption spectra of the disodium diphosphate derivative of natural source lutein in 95% ethanol (λₘₐₓ=446 nm), 95% DMSO (λₘₐₓ=459 nm), and water (λₘₐₓ=428 nm);

FIG. 7 depicts a UV/V is absorption spectra of the disodium diphosphate derivative of natural source lutein in water (λₘₐₓ=428 nm) with increasing concentrations of ethanol;

FIG. 8 depicts a mean percent inhibition (±SEM) of superoxide anion signal as detected by DEPMPO spin-trap by the disodium disuccinate derivative of natural source lutein (tested in water);

FIG. 9 depicts a mean percent inhibition (±SEM) of superoxide anion signal as detected by DEPMPO spin-trap by the disodium diphosphate derivative of natural source lutein (tested in water);

FIG. 10 depicts the chemical structures of three stereoisomers of synthetic water-soluble carotenoid analogs or derivatives according to certain non-limiting embodiments. (A) (3R,3′R)-tetrasodium diphosphate astaxanthin; (B) (3S,3′S)-tetrasodium diphosphate astaxanthin; (C) (3R,3′S; meso)-tetrasodium diphosphate astaxanthin; (D) lycophyll diphosphate

FIG. 11A is a Western blot depicting the levels of CX43 protein in 10T1/2 cells treated with AST versus pAST for four days. FIG. 11B depicts the relative induction levels of CX43 expression versus untreated control;

FIG. 12 depicts various immunofluorescence images of intercellular plaques that are reactive with anti-CX43 antibodies at regions of cell-cell contact. Cells were treated for 4 days as in FIG. 11, then fixed and immuno-stained with CX43 antibody. (A), medium control; (B), pAST 10⁻⁶ M; (C), AST 10⁻⁶ M; (D), CTX 10⁻⁵ M; E, TTNPB, 10⁻⁸ M. Arrows indicate plaque location;

FIG. 13 depicts the comparative induction of GJIC by pAST and AST. Confluent cultures of 10T1/2 cells were treated with the indicated concentrations for 7 days. Communication was assessed by scrape-loading assays as described. ▪-▪, AST; ●-●, pAST. In these cultures a dye-transfer value of 60 is equal to transfer of dye 1 mm from the region of scrape-load.

FIG. 14A is a bar graph and the corresponding Western Blots showing CX43 induction in 10T1/2 cells after treatment according to the following: Lanes: 1: untreated; 2: THF only; 3: TTNPB 10⁻⁸M; 4: lycopene 10⁻⁵ M; 5: lycopene 10⁻⁶ M; 6: lycophyll 10⁻⁵ M; 7: lycophyll 10⁻⁶M; 8: 3S,3′S astaxanthin 10⁻⁵ M; 9: 3S,3′S astaxanthin 10⁻⁶ M;

FIG. 14B is a bar graph showing relative CX43 protein inductions presented as averages of duplicate samples with standard deviation error bars. Bottom Panel (SDS-PAGE/Western Immunodetection). Lanes: 1: THF only; 2: TTNPB 10⁻⁸ M; 3: lycopene 10⁻⁵ M; 4: lycopene 10⁻⁻⁶ M; 5: lycophyll 10⁻⁵ M; 6; lycophyll 10⁻⁶ M; 7; lycophyll 10⁻⁷ M; 8: 3S,3′S astaxanthin 10⁻⁵M; 9: 3S,3′S astaxanthin 10⁻⁶ M; 10: 3S,3′S astaxanthin 10⁻⁷ M;

FIG. 14C shows two bar graphs depicting normalized results of the data shown in FIG. 14A-B;

FIG. 15 is a bar graph depicting the percentage of LNCaP human prostate tumor cells that undergo apoptosis following treatment with various carotenoids including, 3S,3′S-astaxanthin, lycophyll, lycopene and the 5-Lipoxygenase inhibitor MK886 at the indicated concentrations;

FIG. 16 shows a series of flow cytometric profiles indicating the DNA content and approximate cell cycle profile of LNCaP human prostate cancer cells treated with various carotenoids or MK886 for 24 hours in the presence of fetal calf serum. Apoptotic cells are indicated as the population of cells having sub G1 DNA content;

FIG. 17 shows two bar graphs depicting the percentage of LNCaP human prostate tumor cells having 4N DNA content indicating indicating G2/M stage of the cell cycle when treated with various carotenoids or MK886 in the presence of fetal calf serum for 24 hours (upper panel) and 72 hours (lower panel), respectively;

FIG. 18 shows the flow cytometric DNA profiles of LNCaP cells treated with 10 μM MK866 in the absence of fetal calf serum over a timecourse, at the indicated times after treatment from two independent experiments;

FIG. 19 is a bar graph depicting the percentage of LNCaP cells having 4N DNA content 24 hours after being treated with the indicated carotenoid or with MK866 at the indicated concentration;

FIG. 20 shows the DNA profiles of LNCaP cells treated for 24 hours in the presence of serum and the indicated carotenoid or MK866. Each experiment was performed in duplicate as indicated;

FIG. 21 is a bar graph showing the cell cycle stage of LNCaP cells treated with the indicated agents for 24 or 48 hours (upper four panels), and the same results in tabular form.

While the invention is susceptible to various modifications and alternative forms, specific embodiments thereof are shown by way of example in the drawings and will be described herein in detail. It should be understood that the drawings and detailed description attached thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION

Definitions

The terms used throughout this specification generally have their ordinary meanings in the art, within the context of the invention, and in the specific context where each term is used. Certain terms are discussed below, or elsewhere in the specification, to provide additional guidance to the practitioner in describing the devices and methods of the invention and how to make and use them. It will be appreciated that the same thing can be said in more than one way. Consequently, alternative language and synonyms may be used for any one or more of the terms discussed herein, nor is any special significance to be placed upon whether or not a term is elaborated or discussed in greater detail herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term.

As used herein, the term “xanthophyll carotenoid” generally refers to a naturally occurring or synthetic 40-carbon polyene chain with a carotenoid structure that contains at least one oxygen-containing functional group. The chain may include terminal cyclic end groups. Exemplary, though non-limiting, xanthophyll carotenoids include astaxanthin, zeaxanthin, lutein, echinenone, lycophyll, canthaxanthin, and the like. Non-limiting examples of carotenoids that are not xanthophyll carotenoids include β-carotene and lycopene.

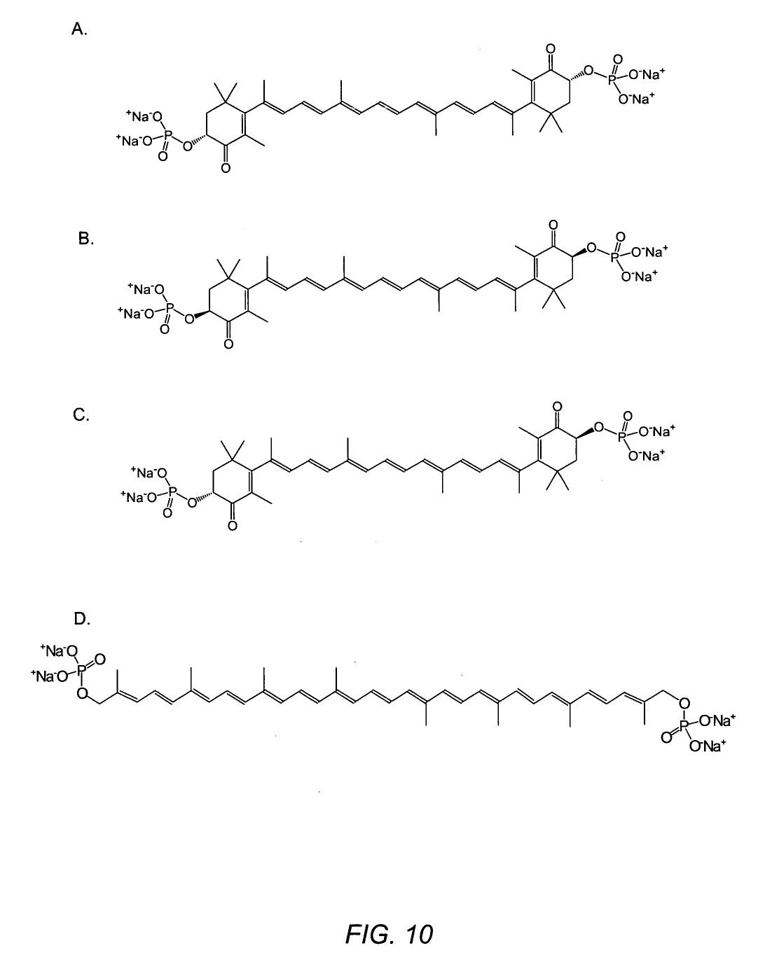

As used herein, terms such as “carotenoid analog” and “carotenoid derivative” generally refer to chemical compounds or compositions derived from a naturally occurring or synthetic carotenoid. Terms such as carotenoid analog and carotenoid derivative may also generally refer to chemical compounds or compositions that are synthetically derived from non-carotenoid based parent compounds; however, which ultimately substantially resemble a carotenoid derived analog. Non-limiting examples of carotenoid analogs and derivatives that may be used according to some of the embodiments described herein are depicted schematically in FIG. 10

The term “modulate,” as used herein, generally refers to a change or an alteration in the magnitude of a be used herein to biological parameter such as, for example, foci formation, tumorigenic or neoplastic potential, apoptosis, growth kinetics, expression of one or more genes or proteins of interest, metabolism, oxidative stress, replicative status, intercellular communication, or the like. “Modulation” may refer to a net increase or a net decrease in the biological parameter.

As used herein, the term “proliferative disorder” generally refers to a disorder, a substantial component of which involves the aberrant (typically accelerated) proliferation or growth of cells. Non-limiting examples of proliferative disorders include chronic inflammatory proliferative disorders, e.g., psoriasis and rheumatoid arthritis; proliferative ocular disorders, e.g., diabetic retinopathy; benign proliferative disorders, e.g., hemangiomas; and cancer, such as neoplasia, lymphoma, sarcoma, melanoma and other malignancies and tumors.

As used herein, the term “cancer” refers to a cellular disorder characterized by uncontrolled or dysregulated cell proliferation, decreased cellular differentiation, inappropriate ability to invade surrounding tissue, and/or ability to establish new growth at ectopic sites. The term “cancer” includes, but is not limited to, solid tumors and bloodborne tumors. The term “cancer” encompasses diseases of skin, tissues, organs, bone, cartilage, blood, and vessels. The term “cancer” further encompasses primary and metastatic cancers.

As used herein, the term “cell or a group of cells” is meant to include a single cell or group of cells that are isolated in culture as well as those cells or groups of cells naturally residing in a body or as part of a body organ or body tissue. The term “organ”, when used in reference to a part of the body of an animal or of a human generally refers to the collection of cells, tissues, connective tissues, fluids and structures that are part of a structure in an animal or a human that is capable of performing some specialized function. Groups of organs constitute one or more specialized body systems. The specialized function performed by an organ is typically essential to the life or the overall well-being of the animal or human. Non-limiting examples of body organs include the heart, lungs, kidney, ureter, urinary bladder, adrenal glands, pituitary gland, skin, prostate, uterus, reproductive organs (e.g., genitalia and accessory organs), liver, gall bladder, brain, spinal cord, stomach, intestine, appendix, pancreas, lymph nodes, breast, salivary glands, lacrimal glands, eyes, spleen, thymus, bone marrow. Non-limiting examples of body systems include the respiratory, circulatory, musculoskeletal, nervous, digestive, endocrine, exocrine, hepato-biliary, reproductive, and urinary systems. In animals the organs are generally made up of several tissues, one of which usually predominates, and determines the principal function of the organ. The term “tissue”, when used in reference to a part of a body or of an organ, generally refers to an aggregation or collection of morphologically similar cells and associated accessory cells and intercellular matter, including extracellular matix material and fluids, acting together to perform specific functions in the body. There are generally four basic types of tissue in animals and humans including muscle, nerve, epithelial, and connective tissues.

The terms “cells” or “groups of cells” as used herein further encompasses cultured cells that have been explanted from a body or tissue and that have been maintained in vitro in a cell culture system. Examples of such cells include “primary cell” cultures. Primary cells are those cells that are explanted directly from a donor organism or tissue. Primary cells may typically be capable of undergoing a limited number of divisions in culture, but they generally do not continue to grow and eventually senesce and die.

Further examples of isolated cells include “secondary cell” cultures. Secondary cells are those cells that are explanted directly from a donor organism or tissue and that are maintained and propagated in culture for a protracted period of time, typically exceeding that of primary cells. Often times, secondary cells may be propagated in vitro for up to as many as 100 generations or more. Secondary cells are typically not immortalized however, and eventually undergo senescence. The number of cell divisions that secondary cells may undergo is related to their degree of differentiation. More terminally differentiated cells undergo fewer cell divisions and senesce early. Less well-differentiated cells, such as embryonic fibroblasts and cells that have begun to undergo neoplastic transformation, typically have a higher generation potential and can undergo a greater number of divisions.

Yet further examples of isolated cells include “immortalized cells.” Immortalized cells may typically be maintained and propagated in vitro indefinitely as long as the correct culture conditions are maintained. Immortalized cell lines are commonly referred to in the art as “transformed cells.” The growth properties of such cells are altered. Typically, such cells have undergone one or more genotypic changes, such as, for example point mutations, aneuploidy or other chromosomal alterations. Immortalized cells may or may not be cancerous or malignant. Non-malignant transformed cells typically exhibit one or more of several properties when grown in vitro. Non-limiting examples of the phenotypic properties exhibited by non-malignant transformed cells include anchorage-dependent growth, growth factor dependence, and growth-arrest under conditions of nutritional deficiency. Furthermore, while transformed cells are generally not as highly differentiated as their primary or secondary counterparts, they nonetheless typically retain at least a subset of the morphological and biochemical properties of the cell type from which they are derived. Finally, non-malignant cells exhibit a growth property known in the art as “contact inhibition.” Typically, such cells will continue to grow and divide in vitro when plated at low density. When the density of cells is sufficient so that a “monolayer” of cells has formed (i.e., the borders of adjacent cells are substantially touching), growth inhibitory signals pass between the cells, the cells exit the cell cycle and cease dividing. Such “contact inhibited” cells are frequently coupled by gap junctions. Loss of contact inhibition is a widely regarded sign that cells have become cancerous or oncogenic. Such cells do not stop dividing when they form a monolayer in culture. Rather, they continue to divide and pile up on top of one another in “foci”. It is generally well accepted by ordinary practitioners of the art that cells that form foci in culture are tumor cells.

As used herein, the term “neoplastic transformation” or “oncogenic transformation,” generally refers to a proliferative disorder of cells characterized by one or more of several cellular changes. Such cellular changes are manifested by cells that have become, or are on the way to becoming, cancerous or malignant. Characteristics of cells that have undergone neoplastic transformation are well known to ordinary practitioners of the art and may include, but are not limited to, loss of contact inhibition, escape from control mechanisms, loss of GJIC, increased growth potential, increased growth rate, the ability to form colonies in soft agar, alterations in the cell surface, alterations in the expression of certain protein or gene markers, karyotypic abnormalities, aneuploidy, morphological and biochemical deviations from the norm, and other attributes that confer the ability of the cell or group of cells to invade, metastasize, and kill. Neoplastic transformation may be induced, at least in part, by exposure of a cell or group of cells to radiation, or to one or more oncogenic agents such as certain viruses or carcinogens. A “carcinogen” as used herein, generally refers to a substance that increases the likelihood that a cell or group of cells begins the process of neoplastic transformation. Carcinogens may include genotoxic agents, also known in the art as “mutagens”, and non-genotoxic agents, which induce neoplasms by non-genomic mechanisms.

As used herein, a “gap junction” generally refers to a specialized type of intercellular transmembrane protein channel that allows the direct exchange of small molecules (typically with a molecular mass not exceeding about 1.2 kDa) between adjacent cells. Gap junctions are comprised of two hemichannels (commonly referred to in the art as “connexons”), one of each of which spans the apposing membrane of adjacent cells, and that associate to form a unitary intercellular channel. Connexons, in turn, are formed by the oligomerization of at least six protein subunits, termed connexins (Cx). Connexons allow for the electrical coupling of adjacent cells, such as, for example, cardiomyocytes. Additionally, small molecules and ions such as, for example, water, salts, small organic or inorganic ions, mono- and oligosaccharides, amino acids, oligopeptides, nucleotides, and some second messenger molecules (e.g., calcium, cAMP, inositol triphosphate, and the like), or other small molecule signaling mediators can diffuse from the cytoplasm of one cell to that of an adjacent cell through the roughly 1.2 nm wide pore traversing the channel. Macromolecules, such as polypeptides, complex lipids, and polysaccharides and polynucleotides are typically too large to pass through gap junctions and are retained in the cytosol. The exchange of molecules between substantially adjacent cells via gap junctions is generally referred to herein as “gap junctional intercellular communication” (GJIC). A major biological role of GJIC is the maintenance of tissue homeostasis and proliferation, the regulation of embryonic development and differentiation, and electric coupling of electrically excitable cells such as cardiomyocytes. Recently, it has been recognized that intracellular and intercellular signaling via and through gap junctions and their component connexins plays a major role in the regulation of cell division. GJIC may be regulated at several levels, including transcription and translation of connexin genes and mRNA, regulating the processing and stability of connexin mRNA, post-translational modification of connexins, connexon assembly, trafficking, and docking, gating of the gap junction channel, and regulation of the configuration of connexons in an “open” or “closed” configuration.

As used herein, the term “connexin” generally refers to a group of homologous proteins that form the intermembrane channels of gap junctions. Connexins are the major structural and functional proteins of the connexon and gap junctions. Connexin 43 (Cx43), a 43 kDa protein translated from the GLA1 gene transcript, plays an important role in regulating gap junction function. Carboxy-terminal hyperphosphorylation of Cx43 is correlated with incorporation of the protein into functional gap junctions. Reduced cellular expression of certain connexin proteins is correlated with loss of growth inhibitory control and increased malignant potential or neoplastic cells.

As used herein, the term “lipoxygenase” or “LO” generally refers to a class of enzymes that catalyze the oxidative conversion of arachidonic acid to the hydroxyeicosetrinoic acid (HETE) structure in the synthesis of leukotrienes. The term “5-lipoxygenase”, or “5-LO” generally refers to one member of this class of enzymes that has lipoxygenase and dehydrase activity, and that catalyzes the conversion of arachidonic acid to 5-hydroperoxyeicatetraenoic acid (HPETE). 5-HPETE can then be converted to 5-HETE and/or various leukotrienes (e.g., leukotriene A₄ (LTA₄)) that can cause inflammation and asthmatic constriction of the bronchioles in one important pathological human setting. Leukotrienes participate in numerous physiological processes, which may include host defense reactions and pathophysiological conditions such as immediate hypersensitivity and inflammation. Leukotrienes may have potent actions on many essential organs and systems, which may include the cardiovascular, pulmonary, and central nervous system as well as the gastrointestinal tract and the immune system. 5-LO requires the presence of the membrane protein 5-Lipoxygenase-activating protein (FLAP, also known as arachidonate 5-lipoxygenase-activating protein, arachidonate 5-lipoxygenase activating protein, and ALOX5AP). FLAP binds arachidonate, facilitating its interaction with the 5-LO. 5-LO, FLAP, and Phospholipase A₂ (which catalyzes release of arachidonate from phospholipids) form a complex in association with the nuclear envelope during leukotriene synthesis in leukocytes. The activity of 5-LO may be reduced or inhibited in cells by contacting the cell with one or more 5-LO inhibitors. The term “5-lipoxygenase inhibitor” or “5-LO inhibitor” includes any agent or compound that inhibits, restrains, retards or otherwise interacts with the enzymatic action of 5-lipoxygenase, such as, but not limited to, zileuton, docebenone, piripost, and the like. The activity of 5-LO may also be reduced or inhibited in cells by contacting the cell with a FLAP inhibitor. The term “5-lipoxygenase activating protein inhibitor” or “FLAP inhibitor” includes any agent or compound that inhibits, restrains, retards or otherwise interacts with the action or activity of 5-lipoxygenase activating protein including but not limited to the association thereof with 5-LO. Exemplary FLAP inhibitors include the agents MK-591 and MK-886. It is within the skill level of a practioner having ordinary skill in the art to recognize FLAP inhibitors.

The term “apoptosis,” as used herein, generally refers to a morphologically distinct form of programmed cell death that is important in the normal development and maintenance of multicellular organisms. Dysregulation of apoptosis can take the form of inappropriate suppression of cell death, as occurs in the development of cancers, or in a failure to control the extent of cell death, as is believed to occur in acquired immunodeficiency and certain neurodegenerative disorders. Apoptosis is an active process in which cells induce their self-destruction in response to specific cell death signals or in the absence of cell survival signals. It is distinct from necrosis, which is cell death occurring as a result of severe injurious changes in the environment. Apoptosis of a cell can be characterized at least by the rapid condensation of the cell with collapse of the nucleus but preservation of membranes; or, cleavage of nuclear DNA at the linker regions between nucleosomes to produce fragments which can be easily visualized by agarose gel electrophoresis as a characteristic ladder pattern. Cells undergoing apoptosis exhibit a characteristic series of morphological changes including mitochondrial membrane swelling and rupture, leakage of cytosolic contents into the surrounding area, and inflammation in tissues. The pattern of events occurring during apoptosis is orderly and includes; cell shrinkage; appearance of bubble-like blebs on their surface; degradation of chromatin (DNA and protein) in their nucleus; mitochondrial rupture and release of cytochrome c into the cytosol; breakage of the cell into small, membrane-wrapped, fragments (commonly referred to as “apoptotic bodies” or “corpses”); exposure of phosphatidylserine on the outer leaflet of the cell membrane; and recruitment of phagocytic cells like macrophages and dendritic cells which then engulf the cell fragments.

Various pathologies occur due to a defective or aberrant regulation of apoptosis in the affected cells of an organism. For example, defects that result in a decreased level of apoptosis in a tissue as compared to the normal level required to maintain the steady-state of the tissue can promote an abnormal increase of the amount of cells in a tissue. This has been observed in various cancers, where the formation of tumors occurs because the cells are not dying at their normal rate.

As used herein, terms such as “pharmaceutical composition,” “pharmaceutical formulation,” “pharmaceutical preparation,” or the like, generally refer to formulations that are adapted to deliver a prescribed dosage of one or more pharmacologically active compounds to a cell, a group of cells, an organ or tissue, an animal or a human. The determination of an appropriate prescribed dosage of a pharmacologically active compound to include in a pharmaceutical composition in order to achieve a desired biological outcome is within the skill level of an ordinary practitioner of the art. Pharmaceutical preparations may be prepared as solids, semi-solids, gels, hydrogels, liquids, solutions, suspensions, emulsions, aerosols, powders, or combinations thereof. Included in a pharmaceutical preparation may be one or more carriers, preservatives, flavorings, excipients, coatings, stabilizers, binders, solvents and/or auxiliaries. Methods of incorporating pharmacologically active compounds into pharmaceutical preparations are widely known in the art.

As used herein, the term “organ”, when used in reference to a part of the body of an animal or of a human generally refers to the collection of cells, tissues, connective tissues, fluids and structures that are part of a structure in an animal or a human that is capable of performing some specialized physiological function. Groups of organs constitute one or more specialized body systems. The specialized function performed by an organ is typically essential to the life or to the overall well-being of the animal or human. Non-limiting examples of body organs include the heart, lungs, kidney, ureter, urinary bladder, adrenal glands, pituitary gland, skin, prostate, uterus, reproductive organs (e.g., genitalia and accessory organs), liver, gall-bladder, brain, spinal cord, stomach, intestine, appendix, pancreas, lymph nodes, breast, salivary glands, lacrimal glands, eyes, spleen, thymus, bone marrow. Non-limiting examples of body systems include the respiratory, circulatory, cardiovascular, lymphatic, immune, musculoskeletal, nervous, digestive, endocrine, exocrine, hepato-biliary, reproductive, and urinary systems. In animals, the organs are generally made up of several tissues, one of which usually predominates, and determines the principal function of the organ.

As used herein, the term “tissue”, when used in reference to a part of a body or of an organ, generally refers to an aggregation or collection of morphologically similar cells and associated accessory and support cells and intercellular matter, including extracellular matrix material, vascular supply, and fluids, acting together to perform specific functions in the body. There are generally four basic types of tissue in animals and humans including muscle, nerve, epithelial, and connective tissues.

The terms “reducing,” “inhibiting” and “ameliorating,” as used herein, when used in the context of modulating a pathological or disease state, generally refers to the prevention and/or reduction of at least a portion of the negative consequences of the disease state. When used in the context of an adverse side effect associated with the administration of a drug to a subject, the term(s) generally refer to a net reduction in the severity or seriousness of said adverse side effects.

As used herein, the term “systemically,” when used in the context of a physiological parameter, generally refers to a parameter that affects the entire body of a subject, or to a particular body system.

As used herein the terms “administration,” “administering,” or the like, when used in the context of providing a pharmaceutical or nutraceutical composition to a subject generally refers to providing to the subject one or more pharmaceutical, “over-the-counter” (OTC) or nutraceutical compositions in combination with an appropriate delivery vehicle by any means such that the administered compound achieves one or more of the intended biological effects for which the compound was administered. By way of non-limiting example, a composition may be administered parenteral, subcutaneous, intravenous, intracoronary, rectal, intramuscular, intra-peritoneal, transdermal, or buccal routes of delivery. Alternatively, or concurrently, administration may be by the oral route. The dosage administered will be dependent upon the age, health, weight, and/or disease state of the recipient, kind of concurrent treatment, if any, frequency of treatment, and/or the nature of the effect desired. The dosage of pharmacologically active compound that is administered will be dependent upon multiple factors, such as the age, health, weight, and/or disease state of the recipient, concurrent treatments, if any, the frequency of treatment, and/or the nature and magnitude of the biological effect that is desired.

As used herein, the term “treat” generally refers to an action taken by a caregiver that involves substantially inhibiting, slowing or reversing the progression of a disease, disorder or condition, substantially ameliorating clinical symptoms of a disease disorder or condition, or substantially preventing the appearance of clinical symptoms of a disease, disorder or condition.

As used herein, terms such as “pharmaceutical composition,” “pharmaceutical formulation,” “pharmaceutical preparation,” or the like, generally refer to formulations that are adapted to deliver a prescribed dosage of one or more pharmacologically active compounds to a cell, a group of cells, an organ or tissue, an animal or a human. Methods of incorporating pharmacologically active compounds into pharmaceutical preparations are widely known in the art. The determination of an appropriate prescribed dosage of a pharmacologically active compound to include in a pharmaceutical composition in order to achieve a desired biological outcome is within the skill level of an ordinary practitioner of the art. A pharmaceutical composition may be provided as sustained-release or timed-release formulations. Such formulations may release a bolus of a compound from the formulation at a desired time, or may ensure a relatively constant amount of the compound present in the dosage is released over a given period of time. Terms such as “sustained release” or “timed release” and the like are widely used in the pharmaceutical arts and are readily understood by a practitioner of ordinary skill in the art. Pharmaceutical preparations may be prepared as solids, semi-solids, gels, hydrogels, liquids, solutions, suspensions, emulsions, aerosols, powders, or combinations thereof. Included in a pharmaceutical preparation may be one or more carriers, preservatives, flavorings, excipients, coatings, stabilizers, binders, solvents and/or auxiliaries that are, typically, pharmacologically inert. It will be readily appreciated by an ordinary practitioner of the art that, pharmaceutical compositions, formulations and preparations may include pharmaceutically acceptable salts of compounds. It will further be appreciated by an ordinary practitioner of the art that the term also encompasses those pharmaceutical compositions that contain an admixture of two or more pharmacologically active compounds, such compounds being administered, for example, as a combination therapy.

The term “pharmaceutically acceptable salts” includes salts prepared from by reacting pharmaceutically acceptable non-toxic bases or acids, including inorganic or organic bases, with inorganic or organic acids. Pharmaceutically acceptable salts may include salts derived from inorganic bases include aluminum, ammonium, calcium, copper, ferric, ferrous, lithium, magnesium, manganic salts, manganous, potassium, sodium, zinc, etc. Examples include the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-dibenzylethylenediamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, etc.

As used herein the terms “subject” generally refers to a mammal, and in particular to a human.

Terms such as “in need of treatment,” “in need thereof,” “benefit from such treatment,” and the like, when used in the context of a subject being administered a pharmacologically active composition, generally refers to a judgment made by an appropriate healthcare provider that an individual or animal requires or will benefit from a specified treatment or medical intervention. Such judgments may be made based on a variety of factors that are in the realm of expertise of healthcare providers, but include knowledge that the individual or animal is ill, will be ill, or is at risk of becoming ill, as the result of a condition that may be ameliorated or treated with the specified medical intervention.

By “therapeutically effective amount” is meant an amount of a drug or pharmaceutical composition that will elicit at least one desired biological or physiological response of a cell, a tissue, a system, animal or human that is being sought by a researcher, veterinarian, physician or other caregiver.

By “prophylactically effective amount” is meant an amount of a pharmaceutical composition that will substantially prevent, delay or reduce the risk of occurrence of the biological or physiological event in a cell, a tissue, a system, animal or human that is being sought by a researcher, veterinarian, physician or other caregiver.

The term “pharmacologically inert,” as used herein, generally refers to a compound, additive, binder, vehicle, and the like, that is substantially free of any pharmacologic or “drug-like” activity.

A “pharmaceutically or nutraceutically acceptable formulation,” as used herein, generally refers to a non-toxic formulation containing a predetermined dosage of a pharmaceutical and/or nutraceutical composition, wherein the dosage of the pharmaceutical and/or nutraceutical composition is adequate to achieve a desired biological outcome. The meaning of the term may generally include an appropriate delivery vehicle that is suitable for properly delivering the pharmaceutical composition in order to achieve the desired biological outcome.

As used herein the term “antioxidant” may be generally defined as any of various substances (such as beta-carotene, vitamin C, and α-tocopherol) that inhibit oxidation or reactions promoted by Reactive Oxygen Species (ROS) and other radical and non-radical species.

As used herein the term “co-antioxidant” may be generally defined as an antioxidant that is used and that acts in combination with another antioxidant (e.g., two antioxidants that are chemically and/or functionally coupled, or two antioxidants that are combined and function with each another in a pharmaceutical preparation). The effects of co-antioxidants may be additive (i.e., the anti-oxidative potential of one or more anti-oxidants acting additively is approximately the sum of the oxidative potential of each component anti-oxidant) or synergistic (i.e., the anti-oxidative oxidative potential of one or more anti-oxidants acting synergistically may be greater than the sum of the oxidative potential of each component anti-oxidant).

The terms “Rⁿ” in a chemical formula refer to hydrogen or a functional group, each independently selected, unless stated otherwise. In some embodiments the functional group may be an organic group. In some embodiments the functional group may be an alkyl group. In some embodiments, the functional group may be a hydrophobic or hydrophilic group.

Compounds described herein embrace isomers mixtures, racemic, optically active, and optically inactive stereoisomers and compounds.

Inhibition of Neoplastic Transformation by Carotenoid Analogs or Derivatives

Recent studies have demonstrated the utility for modulating cell growth and inhibition of neoplastic transformation of increasing connexin protein expression and GJIC to normal or near-normal levels. In an embodiment, astaxanthin-, lycophyll-, lutein- and zeaxanthin-based supplementation may be used in the therapeutic treatment of proliferative disorders involving dysregulated cell growth and/or neoplastic transformation of cells. The potential utility of these formulations, as well as other carotenoid-based formulations, may be extended for clinical application by providing compounds with sufficient dispersibility in aqueous delivery vehicles so that therapeutic doses of the compounds may be delivered to a subject. Adequate aqueous dispersibility may allow for parenteral administration of carotenoid analogs or derivatives. Parenteral administration may allow for better treating the significant human population of carotenoid oral non-responders as well as acute clinical application(s) requiring rapid loading of therapeutic doses.

In a first set of non-limiting embodiments, methods are provided to increase Cx43 expression in neoplastic cells. Increased expression of Cx43 may be accomplished by contacting a transformed cell or group of cells with a pharmaceutically acceptable composition containing an effective amount of one or more carotenoid analogs or derivatives. In certain embodiments, the carotenoid analog or derivative is an analog or derivative of a xanthophyll carotenoid. In certain embodiments, the carotenoid analog or derivative is an analog or derivative of lycophyll. By way of a non-limiting embodiment, lycophyll analogs or derivatives that may be suited to at least some of the therapeutic applications comtemplated herein may include an analog or derivative of a lycophyll diphosphate. In certain embodiments, the carotenoid analog or derivative is an analog or derivative of astaxanthin. In other embodiments, the carotenoid analog or derivative is an analog or derivative of astaxanthin. By way of a further non-limiting embodiment, astaxanthin analogs or derivatives that may be suited to at least some of the therapeutic applications comtemplated herein may include an analog or derivative of an astaxanthin diphosphate. In certain embodiments, the carotenoid analog or derivative is a salt of an analog or derivative of astaxanthin.

Without being bound by any particular theory of mechanism of action, it is believed that the restoration or maintenance of growth inhibitory mechanisms may result, at least in part, from a restoration of physiologically normal amounts of one or more connexin proteins, such as, for example, connexin 43. As used herein, the term “physiologically normal amounts of” a reference protein, when used in the context of a transformed cell, generally refers to the level of the reference protein that is typically expressed in a non-transformed, or normal, cell of the same lineage as the transformed cell. Restoration of growth inhibitory mechanisms in neoplastic cells may further result from the reestablishment of GJIC between adjacent or substantially adjacent cells. The direct superoxide anion scavenging ability of the carotenoid analogs and derivatives described herein may provide further advantageous health benefits.

The carotenoid analogs or derivatives may be advantageously administered to increase Cx43 expression in a subject in whom a beneficial therapeutic or prophylactic effect can be achieved thereby, i.e., a subject in need of treatment for a proliferative disorder. A “subject” is a mammal, preferably a human or an animal in need of veterinary treatment, e.g., companion animals (e.g., dogs, cats, and the like), farm animals (e.g., cows, sheep, pigs, horses, and the like), and laboratory animals (e.g., rats, mice, guinea pigs, and the like).

Certain carotenoid analogs or derivatives are particularly useful in therapeutic applications relating to a disorder characterized by increased cellular proliferation resulting from reduced or inhibited GJIC. As used herein, the term “GJIC-mediated disorder” includes any disorder, disease or condition whose etiology is due, at least in part, to reduced Cx43 expression or activity, or a reduction in the number of functional gap juntions. The term “GJIC-mediated disorder” also includes any disorder, disease or condition in which increased Cx43 expression and GJIC is beneficial.

Increased Cx43 expression and GJIC resulting from the administration of carotenoid analogs or derivatives may also be used to achieve a beneficial therapeutic or prophylactic effect, for example, in subjects with a proliferative disorder. Non-limiting examples of proliferative disorders include chronic inflammatory proliferative disorders, e.g., psoriasis and rheumatoid arthritis; proliferative ocular disorders, e.g., diabetic retinopathy; benign proliferative disorders, e.g., hemangiomas; and cancer. Non-limiting examples of solid tumors that can be treated with the disclosed carotenoid analogs or derivatives include pancreatic cancer; bladder cancer; colorectal cancer; breast cancer, including metastatic breast cancer; prostate cancer, including androgen-dependent and androgen-independent prostate cancer; renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma.

The disclosed carotenoid analogs or derivatives and treatment methods may be particularly suited to the treatment of cancers or cell types in which Cx43 expression and/or GJIC activity is downregulated, including, without limitation, rapidly proliferating cells and drug-resistant cells, as well as retinoblastomas such as Rb negative or inactivated cells.

Induction of Apoptosis in Neoplastic Cells by Administration of Carotenoid Analogs or Derivatives

It has recently been found that the subject carotenoid analogs and derivatives can function in certain cells as inhibitors of 5-Lipoxygenase. Biochemical analyses indicate that the subject carotenoid analogs and derivatives bind to 5-LO (see for example, U.S. Patent Appl. Publ. No. 2005/0261254 by Lockwood et al., incorporated herein in its entirety). It is known in the art that administering 5-LO inhibitors to certain cancer cells results in induction of apoptosis in a significant proportion of the transformed cells. For example, as discussed above, Ghosh et al. demonstrated that prostate cancer cells treated with the specific 5-LO inhibitor MK-866 underwent massive apoptosis.

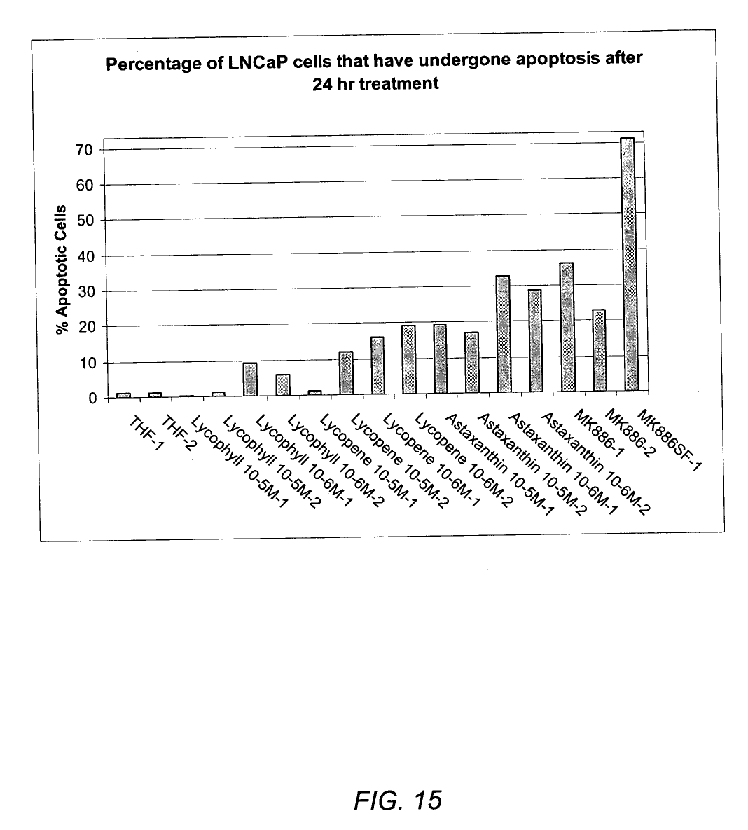

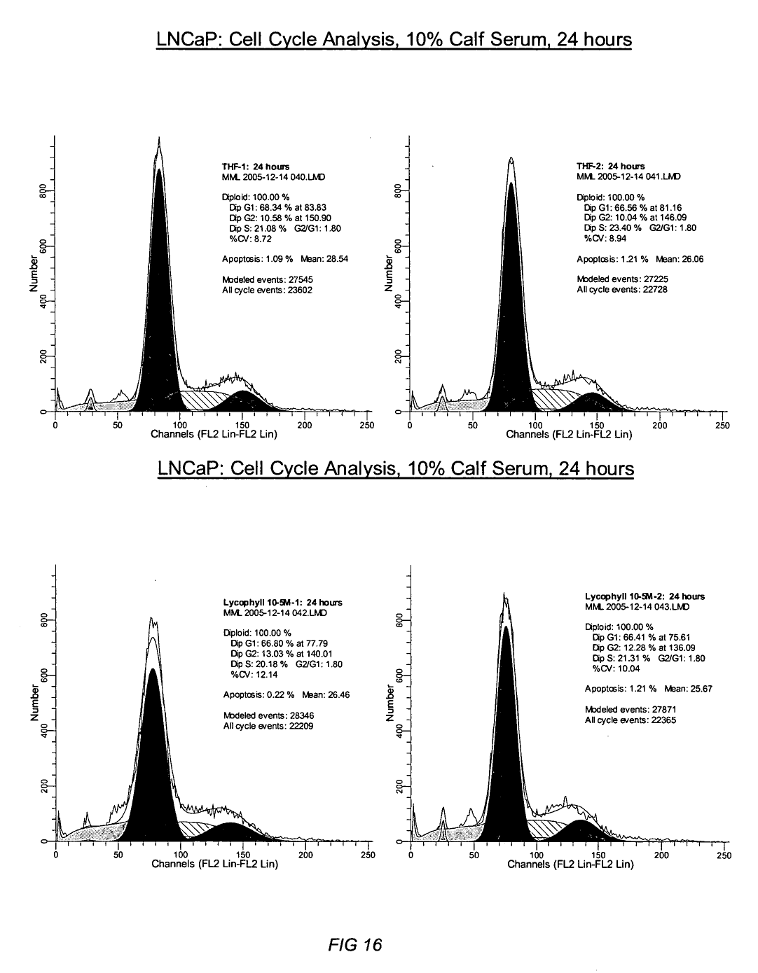

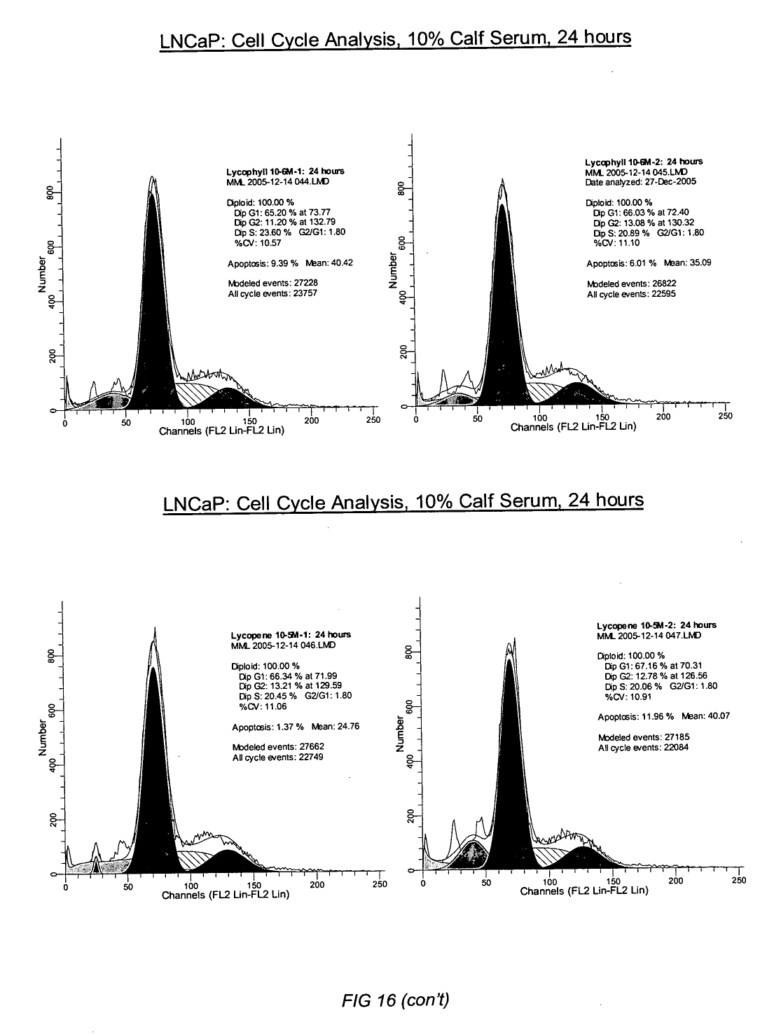

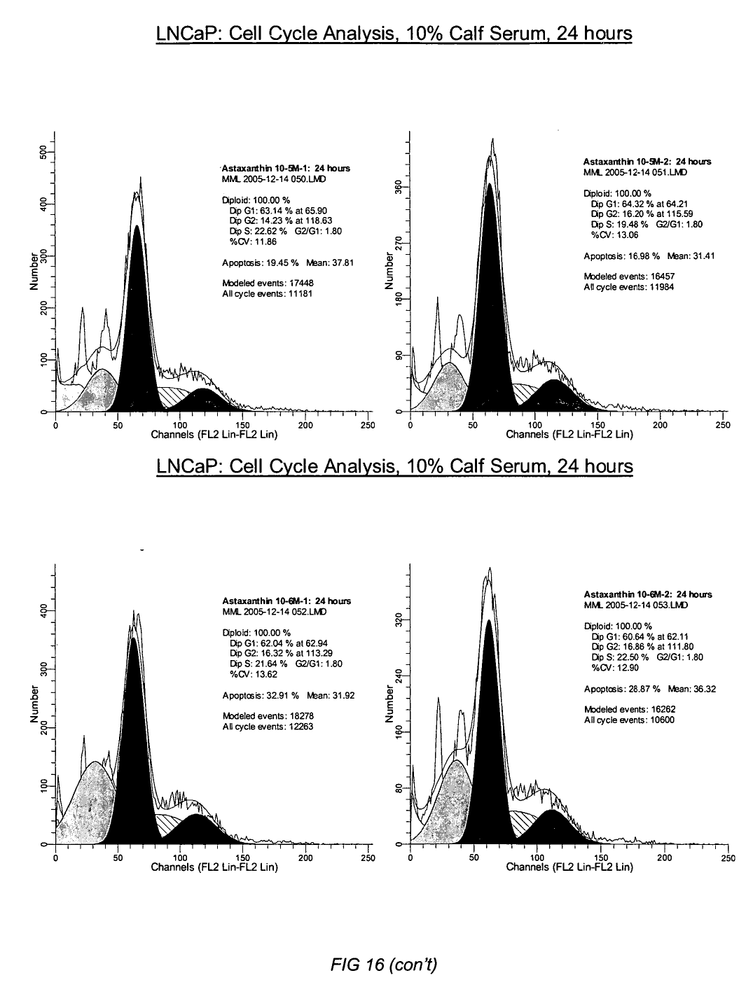

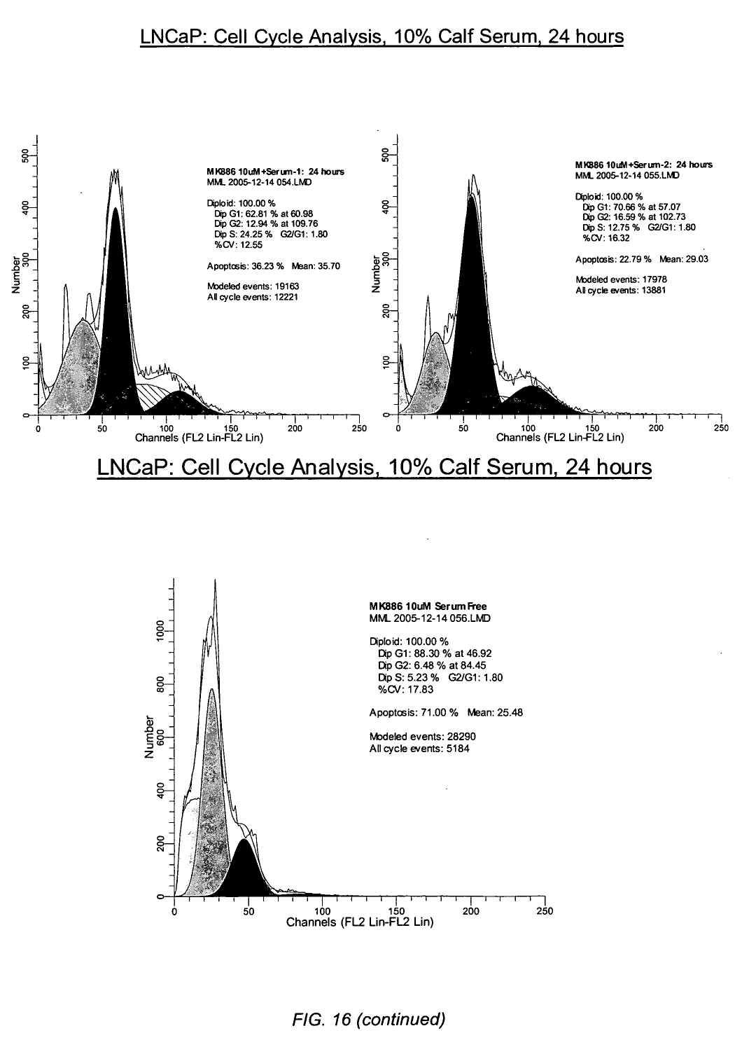

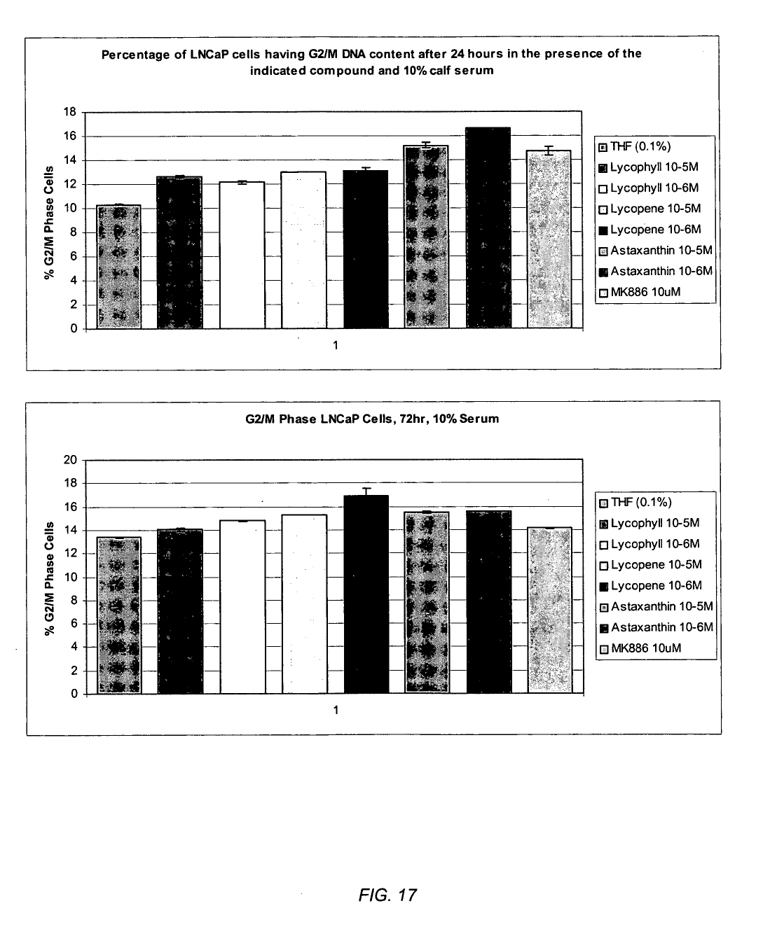

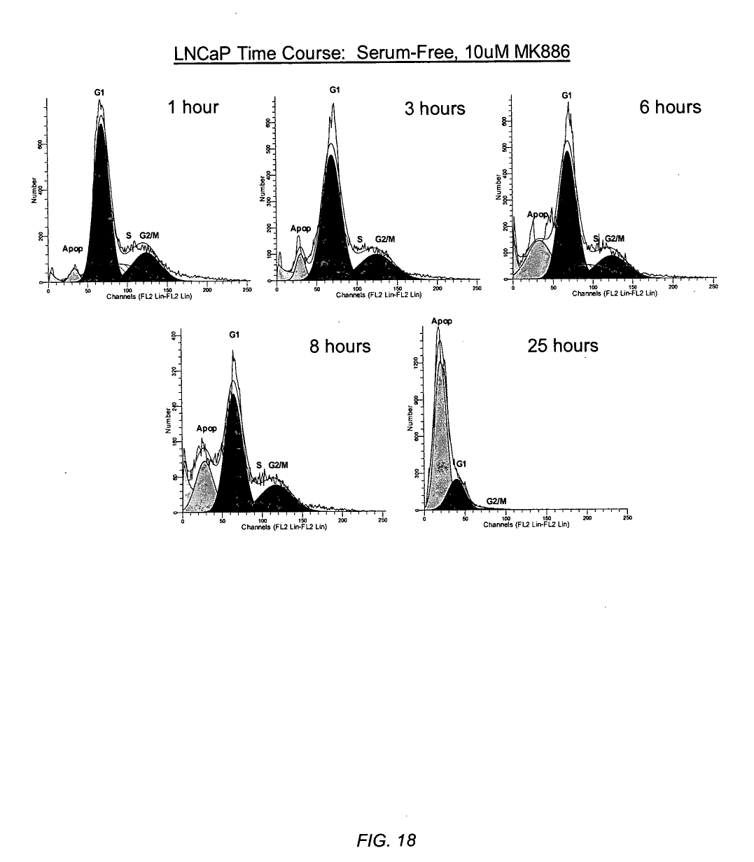

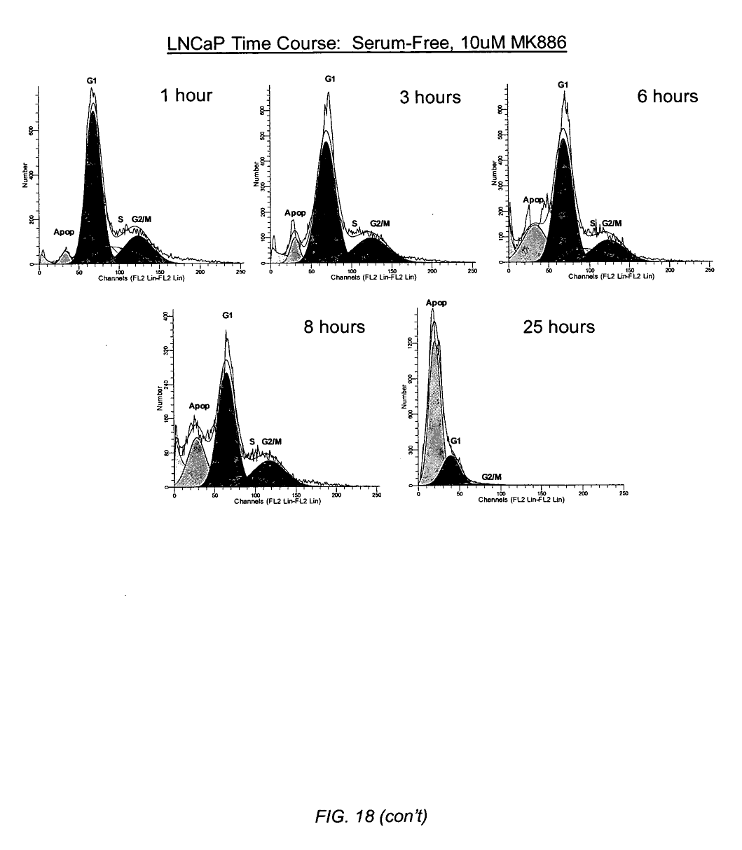

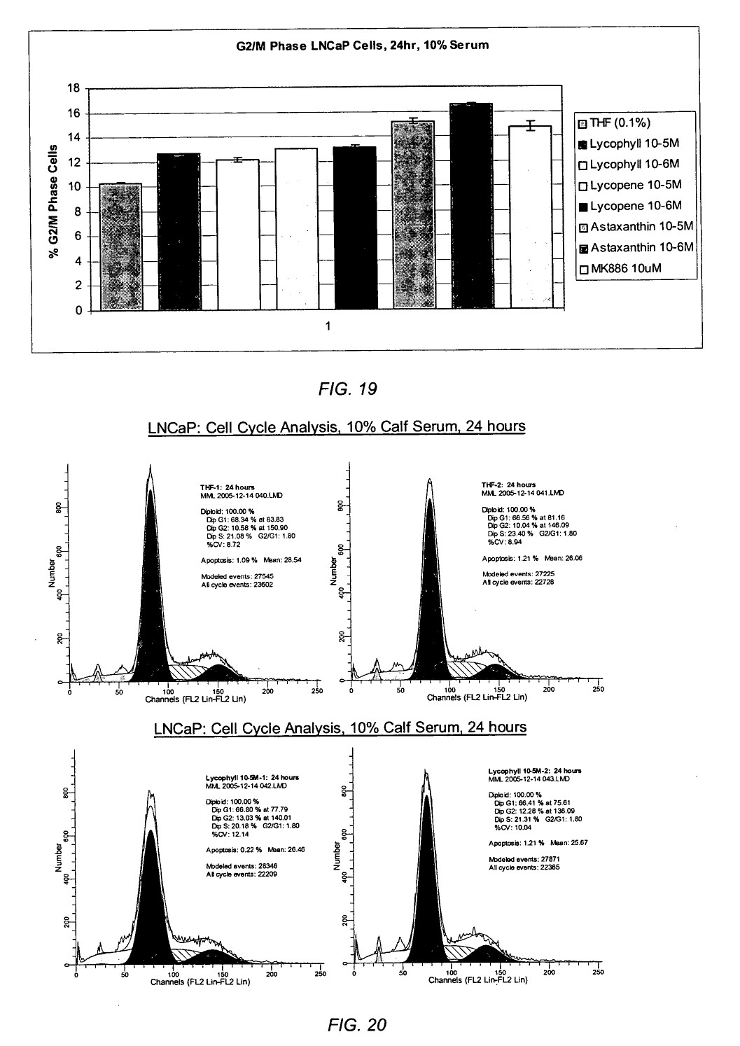

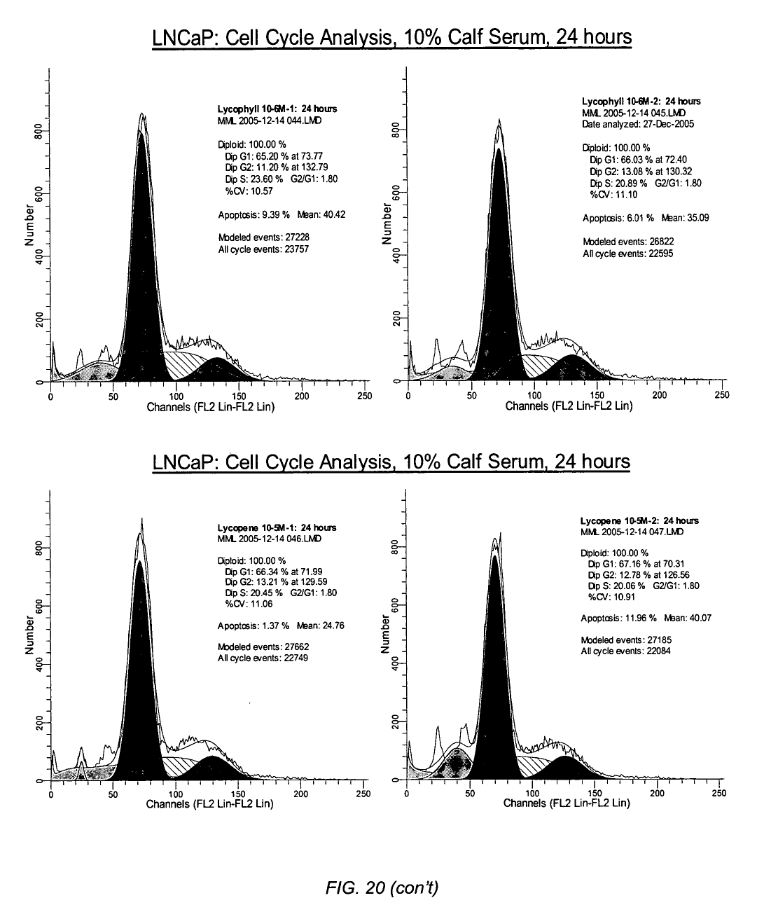

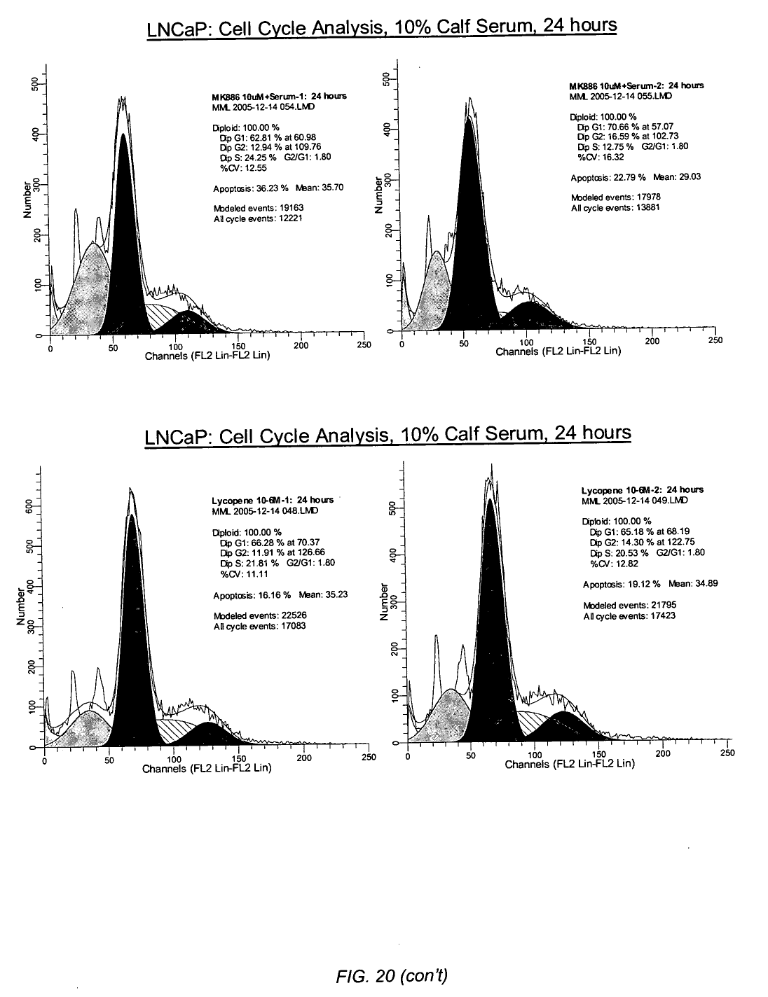

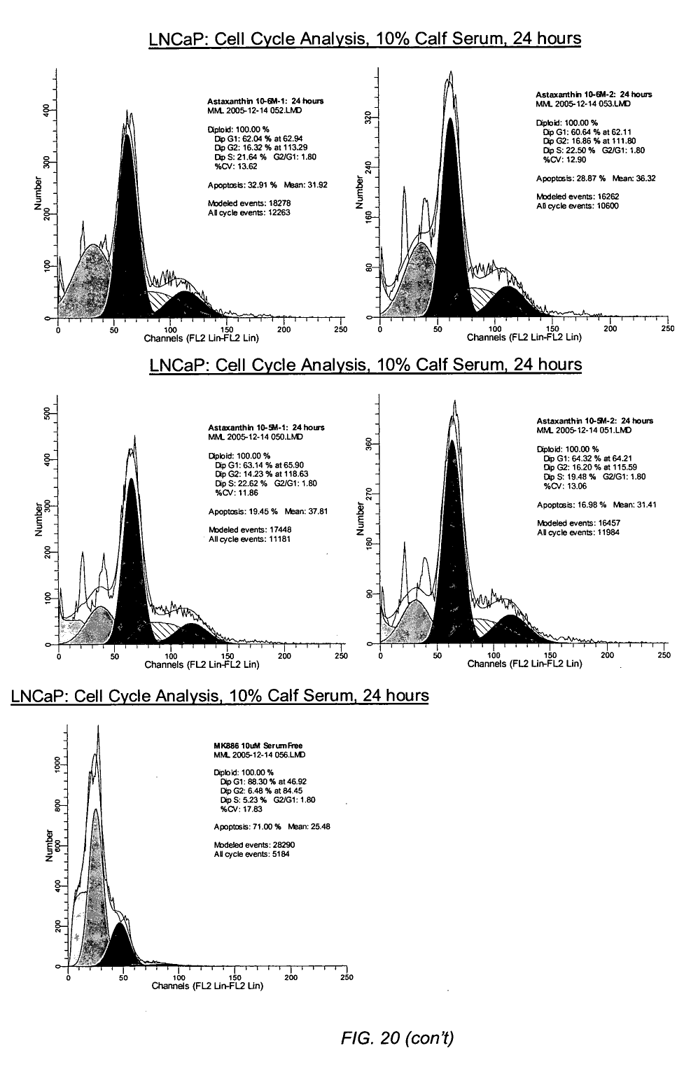

To determine whether the subject carotenoid analogs and derivatives can induce apoptosis in cancer cells, LNCaP human prostate cancer cells were contacted with an effective amount of various carotenoids and xanthophyll carotenoids (over a 2 log concentration range). Induction of apoptosis was measured by flow cytometric and cell cycle analysis of DNA content in propidium iodide-stains cells. Apoptotic cells were identified as those having sub-G1 amounts of DNA (corresponding to apoptotic bodies). The results of these studies are disclosed below and presented in FIGS. 15-20.

Thus, it appears that, in addition to the role of the the subject carotenoid analogs and derivatives in inhibiting the growth of neoplastic cells by enhancing gap junction formation and GJIC, an additional level of cancer treatment may be achieved in certain embodiments, by contacting neoplastic cells with an amount of a composition containing one or more of the subject carotenoid analogs or derivatives sufficient to inhibit 5-LO function and/or induce apoptosis.

Treatment of Proliferative Disorders with Compositions Containing Carotenoid Analogs or Derivatives

In some embodiments, the disclosed carotenoid analogs or derivatives and treatment methods may be used as the sole therapeutic regimen or in conjunction with other therapeutic agents, including anticancer agents. As used herein, the term “anticancer agent” refers to any agent that is administered to a subject with cancer for purposes of treating the cancer. Use of the subject carotenoid analogs or derivatives and methods for the treatment of cancer may be particularly advantageous and may enhance the effectiveness of the anticancer agent when combined with radiation therapy or chemotherapeutic agents that act by causing damage to the genetic material of cells (collectively referred to herein as “DNA damaging agents”); when combined with agents which are otherwise cytotoxic to cancer cells during cell division; when combined with agents which are proteasome inhibitors; when combined with agents which inhibit NF-κB (e.g., IKK inhibitors) (Bottero et al., Cancer Res., 61:7785 (2001)); or used with combinations of cancer drugs with which are not cytotoxic when administered alone, yet in combination produce a toxic effect. Anti-cancer agents having having the properties described above are collectively referred to herein as “chemotherapy agents.” In some non-limiting embodiments, carotenoid analogs or derivatives may be combined with one or more DNA damaging agent and treatment methods.

Non-limiting examples of chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

Chemotherapy agents that disrupt cell replication include: paclitaxel, docetaxel, and related analogs; vincristine, vinblastin, and related analogs; thalidomide and related analogs (e.g., CC-5013 and CC4047); protein tyrosine kinase inhibitors (e.g., imatinib mesylate and gefitinib); antibodies which bind to proteins overexpressed in cancers and thereby downregulate cell replication (e.g., trastuzumab, rituximab, cetuximab, and bevacizumab); and other inhibitors of proteins or enzymes known to be upregulated, over-expressed or activated in cancers, the inhibition of which downregulates cell replication.

The disclosed carotenoid analogs or derivatives and treatment methods are also effective when used in combination with chemotherapy agents and/or radiation therapy to treat subjects with multi-drug resistant cancers. A cancer is resistant to a drug when it resumes a normal rate of tumor growth while undergoing treatment with the drug after the tumor had initially responded to the drug. A tumor “responds to a drug” when it exhibits a decrease in tumor mass or a decrease in the rate of tumor growth. The term “multi-drug resistant cancer” refers to cancer that is resistant to two or more drugs, often as many as five or more.

As such, an “effective amount” of the a carotenoid analog or derivative suitable for the treatment methods described herein is the quantity which increases Cx43 expression and/or induced apoptosis of neoplastic cells when administered to a subject or which, when administered to a subject with cancer, slows tumor growth, ameliorates the symptoms of the disease and/or increases longevity. When used in combination with a chemotherapy agent, an effective amount of the carotenoid analog or derivative is the quantity at which a greater response is achieved when the carotenoid analog or derivative is co-administered with the chemotherapy agents and/or radiation therapy than is achieved when the chemotherapy agent and/or radiation therapy is administered alone. When used as a combination therapy, an “effective amount” of the chemotherapy agents is administered to the subject, which is a quantity that normally produces an anti-cancer effect.

A disclosed carotenoid analog or derivative may be co-administered with another therapeutic chemotheraeutic agent (e.g., DNA-damaging agent, agent that disrupts cell replication, proteasome inhibitor, NF-kB inhibitor, or other anticancer agent) as part of the same pharmaceutical composition or, alternatively, as separate pharmaceutical compositions. When administered separately, carotenoid analog or derivative may be administered prior to, at the same time as, or following administration of the other agent, provided that the enhancing effect on Cx43 expression of the carotenoid analog or derivative is retained.

The amount of carotenoid analog or derivative anti-cancer drug and radiation dose administered to the subject will depend on the type and severity of the disease or condition and on the characteristics of the subject, such as general health, age, sex, body weight and tolerance to drugs. The skilled artisan will be able to determine appropriate dosages depending on these and other factors. Effective dosages for commonly used chemotherapy agents and radiation therapy are well known to the practitioner having ordinary skill in the art.

The carotenoid analog or derivative and treatment methods described herein, and the pharmaceutically acceptable salts, solvates and hydrates thereof may be used in pharmaceutical preparations in combination with a pharmaceutically acceptable carrier or diluent. Suitable pharmaceutically acceptable carriers include inert solid fillers or diluents and sterile aqueous or organic solutions. The carotenoid analog or derivative will typically be present in such pharmaceutical compositions in amounts sufficient to provide the desired dosage amount in the range described herein. Techniques for formulation and administration of the compounds of the instant invention can be found in Remington: the Science and Practice of Pharmacy, 19th edition, Mack Publishing Co., Easton, Pa. (1995).

In some embodiments, carotenoid analogs or derivatives may be employed in “self-formulating” aqueous solutions, in which the compounds spontaneously self-assemble into macromolecular complexes. These complexes may provide stable formulations in terms of shelf-life. The same formulations may be parenterally administered, upon which the spontaneous self-assembly is overcome by interactions with serum and/or tissue components in vivo.

Some specific embodiments may include phosphate derivatives, succinate derivatives, co-antioxidant derivatives (e.g., Vitamin C, Vitamin C analogs, Vitamin C derivatives, Vitamin E, Vitamin E analogs, Vitamin E derivatives, flavonoids, flavonoid analogs, or flavonoid derivatives), or combinations thereof derivatives or analogs of carotenoids. Flavonoids may include, for example, quercetin, xanthohumol, isoxanthohumol, or genistein. Derivatives or analogs may be derived from any known carotenoid (naturally or synthetically derived). Specific examples of naturally occurring carotenoids which compounds described herein may be derived from include for example zeaxanthin, lutein, lycophyll, astaxanthin, and lycopene.

In some embodiments, one or more co-antioxidants may be coupled to a carotenoid or carotenoid derivative or analog.

The synthesis of water-soluble and/or water-dispersible carotenoids (e.g., C₄₀) analogs or derivatives—as potential parenteral agents for clinical applications may improve the injectability of these compounds as therapeutic agents, a result perhaps not achievable through other formulation methods. The methodology may be extended to carotenoids with fewer than 40 carbon atoms in the molecular skeleton and differing ionic character. The methodology may be extended to carotenoids with greater than 40 carbon atoms in the molecular skeleton. The methodology may be extended to non-symmetric carotenoids. The aqueous dispersibility of these compounds allows proof-of-concept studies in model systems (e.g. cell culture), where the high lipophilicity of these compounds previously limited their biovailability and hence proper evaluation of efficacy. Esterification or etherification may be useful to increase oral bioavailabilty, a fortuitous side effect of the esterification process which can increase solubility in gastric mixed micelles. The net overall effect is an improvement in potential clinical utility for the lipophilic carotenoid compounds as therapeutic agents.

In some embodiments, the principles of retrometabolic drug design may be utilized to produce novel soft drugs from the asymmetric parent carotenoid scaffold (e.g., RRR-lutein(β,ε-carotene-3,3′-diol)). For example, lutein scaffold for derivativization was obtained commercially as purified natural plant source material, and was primarily the RRR-stereoisomer (one of 8 potential stereoisomers). Lutein (Scheme 1) possesses key characteristics—similar to starting material astaxanthin—which make it an ideal starting platform for retrometabolic syntheses: (1) synthetic handles (hydroxyl groups) for conjugation, and (2) an excellent safety profile for the parent compound. As stated above, lutein is available commercially from multiple sources in bulk as primarily the RRR-stereoisomer, the primary isomer in the human diet and human retinal tissue.

In some embodiments, carotenoid analogs or derivatives may have increased water solubility and/or water dispersibility relative to some or all known naturally occurring carotenoids. Contradictory to previous research, improved results are obtained with derivatized carotenoids relative to the base carotenoid, wherein the base carotenoid is derivatized with substituents including hydrophilic substituents and/or co-antioxidants.

In some embodiments, the carotenoid derivatives may include compounds having a structure including a polyene chain (i.e., backbone of the molecule). The polyene chain may include between about 5 and about 15 unsaturated bonds. In certain embodiments, the polyene chain may include between about 7 and about 12 unsaturated bonds. In some embodiments a carotenoid derivative may include 7 or more conjugated double bonds to achieve acceptable antioxidant properties.

In some embodiments, decreased antioxidant properties associated with shorter polyene chains may be overcome by increasing the dosage administered to a subject or patient.

In some embodiments, a chemical compound including a carotenoid derivative or analog may have the general structure (126): 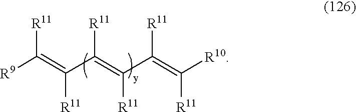
Each R¹¹ may be independently hydrogen or methyl. R⁹ and R¹⁰ may be independently H, an acyclic alkene with one or more substituents, or a cyclic ring including one or more substituents. y may be 5 to 12. In some embodiments, y may be 3 to 15. In certain embodiments, the maximum value of y may only be limited by the ultimate size of the chemical compound, particularly as it relates to the size of the chemical compound and the potential interference with the chemical compound's biological availability as discussed herein. In some embodiments, substituents may be at least partially hydrophilic. These carotenoid derivatives may be included in a pharmaceutical composition.

In some embodiments, the carotenoid derivatives may include compounds having the structure (128): 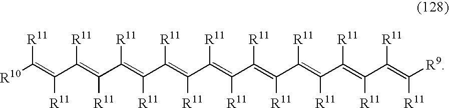
Each R¹¹ may be independently hydrogen, methyl, alkyl, alkenyl, or aromatic substituents. R⁹ and R¹⁰ may be independently H, an acyclic alkene with at least one substituent, or a cyclic ring with at least one substituent having general structure (130): 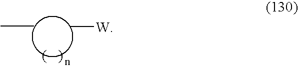
where n may be between 4 to 10 carbon atoms. W is the substituent.

In some embodiments, each cyclic ring may be independently two or more rings fused together to form a fused ring system (e.g., a bi-cyclic system). Each ring of the fused ring system may independently contain one or more degrees of unsaturation. Each ring of the fused ring system may be independently aromatic. Two or more of the rings forming the fused ring system may form an aromatic system.

In some embodiments, a chemical composition may include a carotenoid derivative having the structure 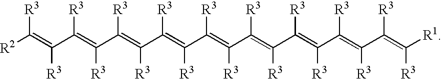
Each R³ may be independently hydrogen or methyl. R¹ and R² may be a cyclic ring including at least one substituent. Each cyclic ring may be independently: 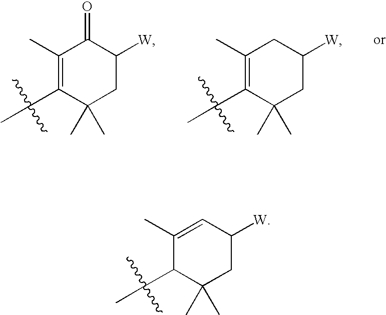
W is the substituent. In some embodiments R¹ and R² may be an acyclic group including at least one substituent. Each acyclic may be:

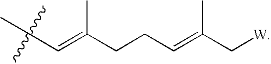

In some embodiments, a chemical composition may include a carotenoid derivative having the structure 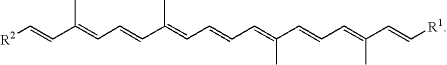
R¹ and R² may be a cyclic ring including at least one substituent. Each cyclic ring may be independently: 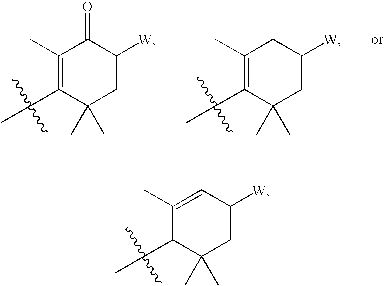
where W is the substituent. In some embodiments R¹ and R² may be an acyclic group including at least one substituent. Each acyclic group may be:

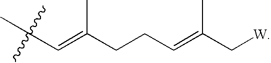

In some embodiments, a method of treating a proliferative disorder may include administering to the subject an effective amount of a pharmaceutically acceptable formulation including a synthetic analog or derivative of a carotenoid. The synthetic analog or derivative of the carotenoid may have the structure 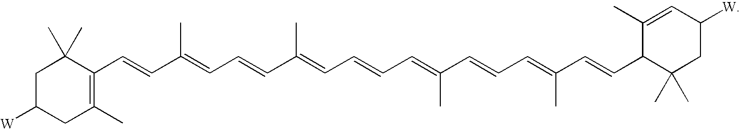
At least one substituent W may independently include 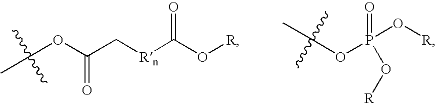
or a co-antioxidant. Each R′ may be CH₂. n may range from 1 to 9. Each R may be independently H, alkyl, aryl, benzyl, alkali metal, or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid analogs, or flavonoid derivatives. Flavonoids may include, for example, quercetin, xanthohumol, isoxanthohumol, or genistein.

Vitamin E may generally be divided into two categories including tocopherols having a general structure 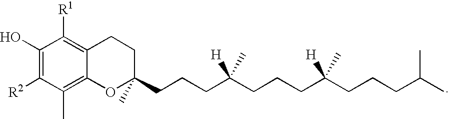
Alpha-tocopherol is used to designate when R¹═R²═CH₃. Beta-tocopherol is used to designate when R¹═CH₃ and R²═H. Gamma-tocopherol is used to designate when R¹═H and R²═CH₃. Delta-tocopherol is used to designate when R¹═R²═H.

The second category of Vitamin E may include tocotrienols having a general structure 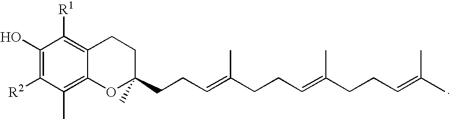
Alpha-tocotrienol is used to designate when R¹═R²═CH₃. Beta-tocotrienol is used to designate when R¹═CH₃ and R²═H. Gamma-tocotrienol is used to designate when R¹═H and R²═CH₃. Delta-tocotrienol is used to designate when R¹═R²═H.

Quercetin, a flavonoid, may have the structure

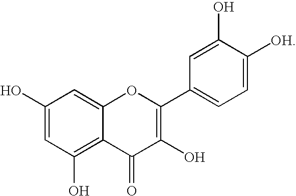

In some embodiments, the carotenoid analog or derivative may have the structure 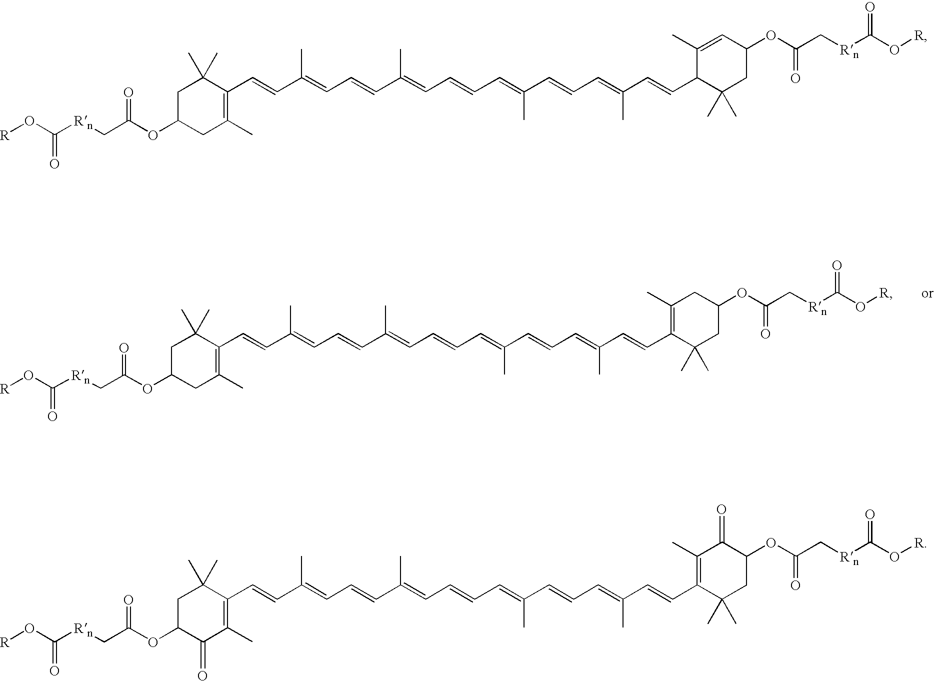
Each R may be independently H, alkyl, aryl, benzyl, alkali metal, or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid analogs, or flavonoid derivatives. Flavonoids may include, for example, quercetin, xanthohumol, isoxanthohumol, or genistein.

In some embodiments, the carotenoid analog or derivative may have the structure 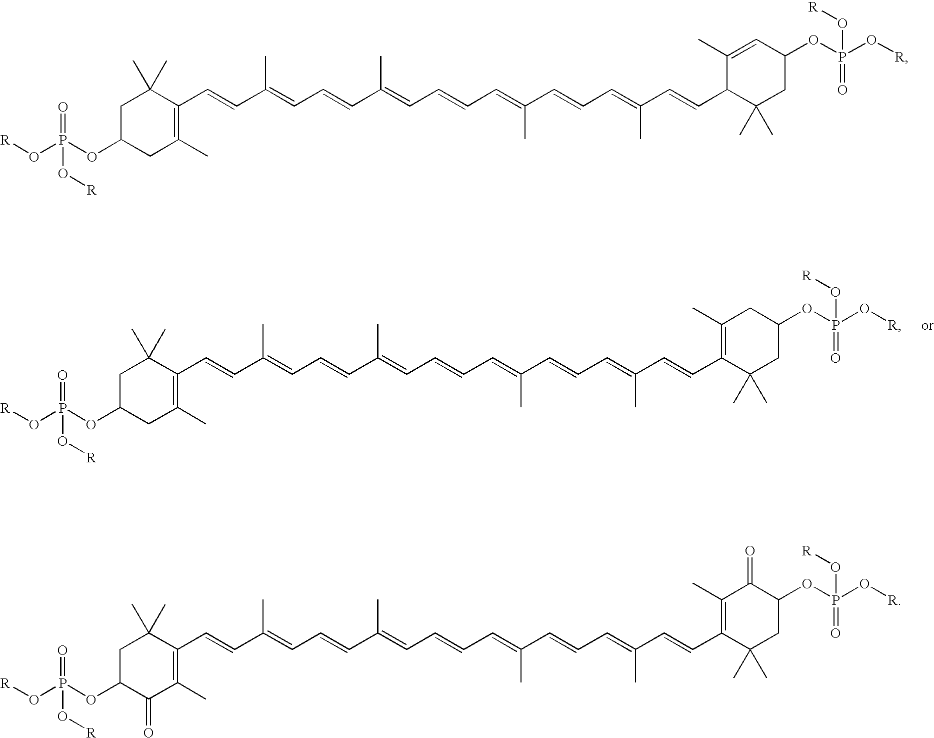
Each R may be independently H, alkyl, aryl, benzyl, alkali metal (e.g., sodium), or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid analogs, or flavonoid derivatives. Flavonoids may include, for example, quercetin, xanthohumol, isoxanthohumol, or genistein. When R includes Vitamin C, Vitamin C analogs, or Vitamin C derivatives, some embodiments may include carotenoid analogs or derivatives having the structure 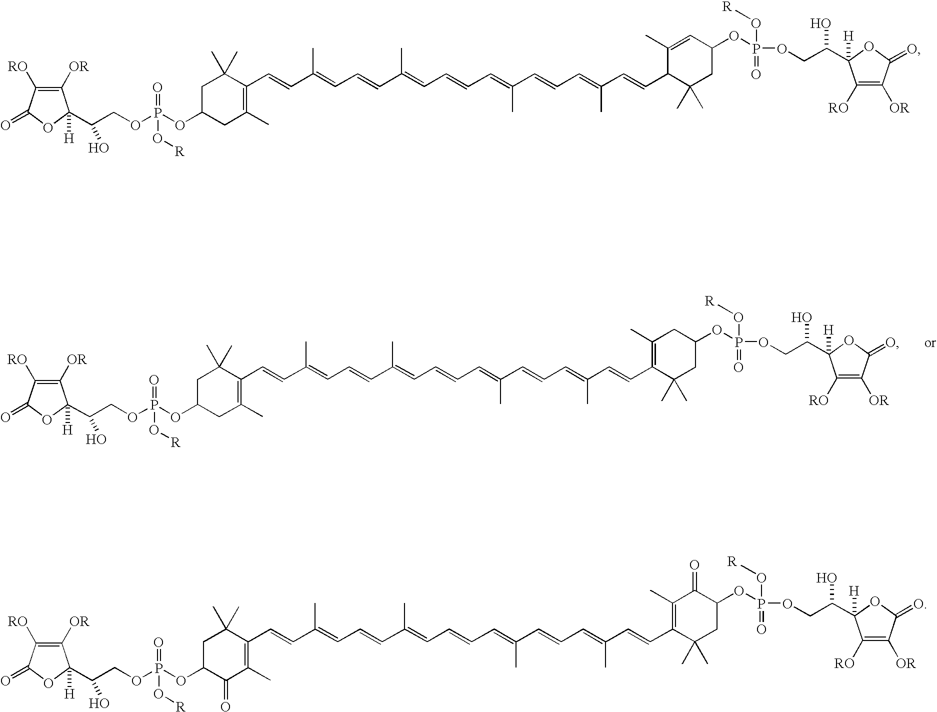
Each R may be independently H, alkyl aryl, benzyl, or alkali metal.

In some embodiments, a chemical compound including a carotenoid derivative may have the general structure (132):

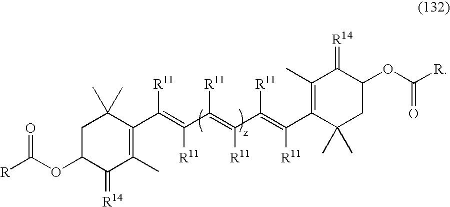

Each R¹¹ may be independently hydrogen or methyl. Each R¹⁴ may be independently O or H₂. Each R may be independently OR¹² or R¹². Each R¹² may be independently -alkyl-NR¹³₃⁺, -aromatic-N R¹³₃⁺, -alkyl-CO₂⁻, -aromatic-CO₂, -amino acid-NH₃⁺, -phosphorylated amino acid-NH₃⁺, polyethylene glycol, dextran, H, alkyl, co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives), or aryl. Each R¹³ may be independently H, alkyl, or aryl. z may range from 5 to 12. In some embodiments, z may range from about 3 to about 15. In certain embodiments, the maximum value of z may only be limited by the ultimate size of the chemical compound, particularly as it relates to the size of the chemical compound and the potential interference with the chemical compound's biological availability as discussed herein. In some embodiments, substituents may be at least partially hydrophilic. These carotenoid derivatives may be used in a pharmaceutical composition.

In some embodiments, a chemical compound including a carotenoid derivative may have the general structure (134): 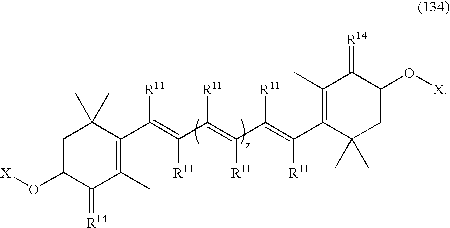
Each R¹¹ may be independently hydrogen or methyl. Each R¹⁴ may be independently O or H₂. Each X may be independently 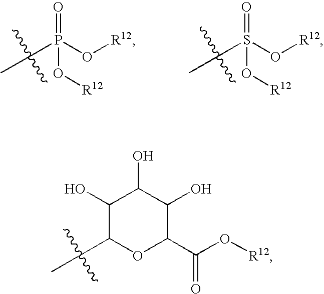
-alkyl-N R¹²₃⁺, -aromatic-NR¹²₃⁺, -alkyl-CO₂⁻, -aromatic-CO₂-, -amino acid-NH₃⁺, -phosphorylated amino acid-NH₃⁺, polyethylene glycol, dextran, alkyl, alkali metal, co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives), or aryl. Each R¹² is independently -alkyl-N R¹³₃⁺, -aromatic-N R¹³₃⁺, -alkyl-CO₂⁻, -aromatic-CO₂^{—}, -amino acid-NH₃⁺, -phosphorylated amino acid-NH₃⁺, polyethylene glycol, dextran, H, alkyl, aryl, benzyl, alkali metal, co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives), or alkali salt. Each R¹³ may be independently H, alkyl, or aryl. z may range from 5 to 12. In some embodiments, z may range from about 3 to about 15. In certain embodiments, the maximum value of z may only be limited by the ultimate size of the chemical compound, particularly as it relates to the size of the chemical compound and the potential interference with the chemical compound's biological availability as discussed herein. In some embodiments, substituents may be at least partially hydrophilic. These carotenoid derivatives may be used in a pharmaceutical composition.

In some non-limiting examples, five- and/or six-membered ring carotenoid derivatives may be more easily synthesized. Synthesis may come more easily due to, for example, the natural stability of five- and six-membered rings. Synthesis of carotenoid derivatives including five- and/or six-membered rings may be more easily synthesized due to, for example, the availability of naturally-occurring carotenoids including five- and/or six-membered rings. In some embodiments, five-membered rings may decrease steric hindrance associated with rotation of the cyclic ring around the molecular bond connecting the cyclic ring to the polyene chain. Reducing steric hindrance may allow greater overlap of any π oribitals within a cyclic ring with the polyene chain, thereby increasing the degree of conjugation and effective chromophore length of the molecule. This may have the salutatory effect of increasing antioxidant capacity of the carotenoid derivatives.

In some embodiments, a substituent (W) may be at least partially hydrophilic. A hydrophilic substituent may assist in increasing the water solubility of a carotenoid derivative. In some embodiments, a carotenoid derivative may be at least partially water-soluble. The cyclic ring may include at least one chiral center. The acyclic alkene may include at least one chiral center. The cyclic ring may include at least one degree of unsaturation. In some cyclic ring embodiments, the cyclic ring may be aromatic. One or more degrees of unsaturation within the ring may assist in extending the conjugation of the carotenoid derivative. Extending conjugation within the carotenoid derivative may have the salutatory effect of increasing the antioxidant properties of the carotenoid derivatives. In some embodiments, the substituent W may include, for example, a carboxylic acid, an amino acid, an ester, an alkanol, an amine, a phosphate, a succinate, a glycinate, an ether, a glucoside, a sugar, or a carboxylate salt.

In some embodiments, each substituent —W may independently include —XR. Each X may independently include O, N, or S. In some embodiments, each substituent —W may independently comprises amino acids, esters, carbamates, amides, carbonates, alcohol, phosphates, or sulfonates. In some substituent embodiments, the substituent may include, for example (d) through (uu): 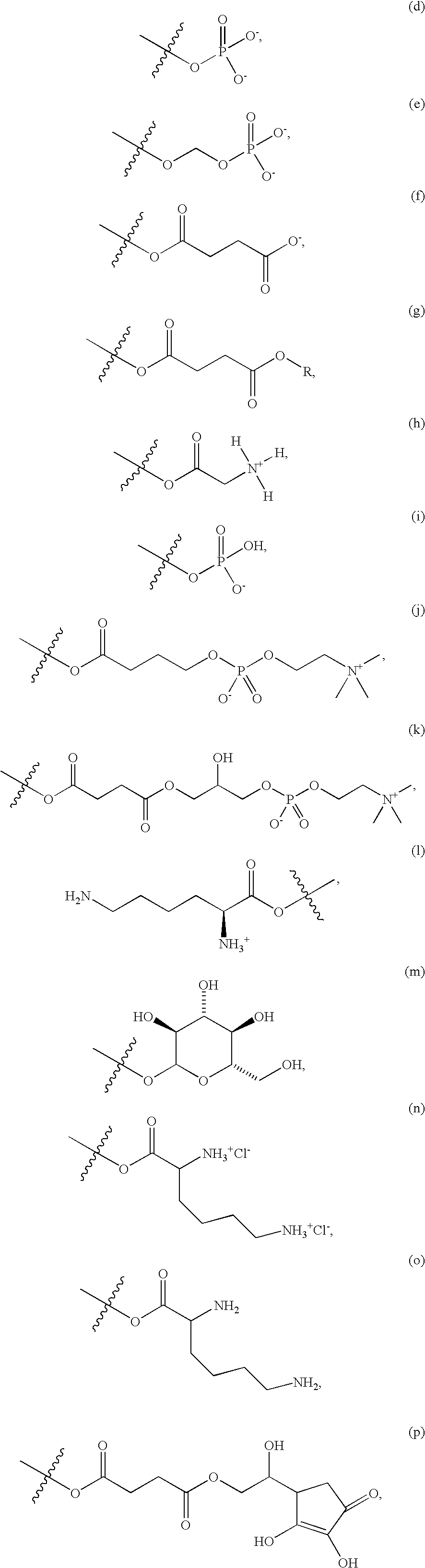 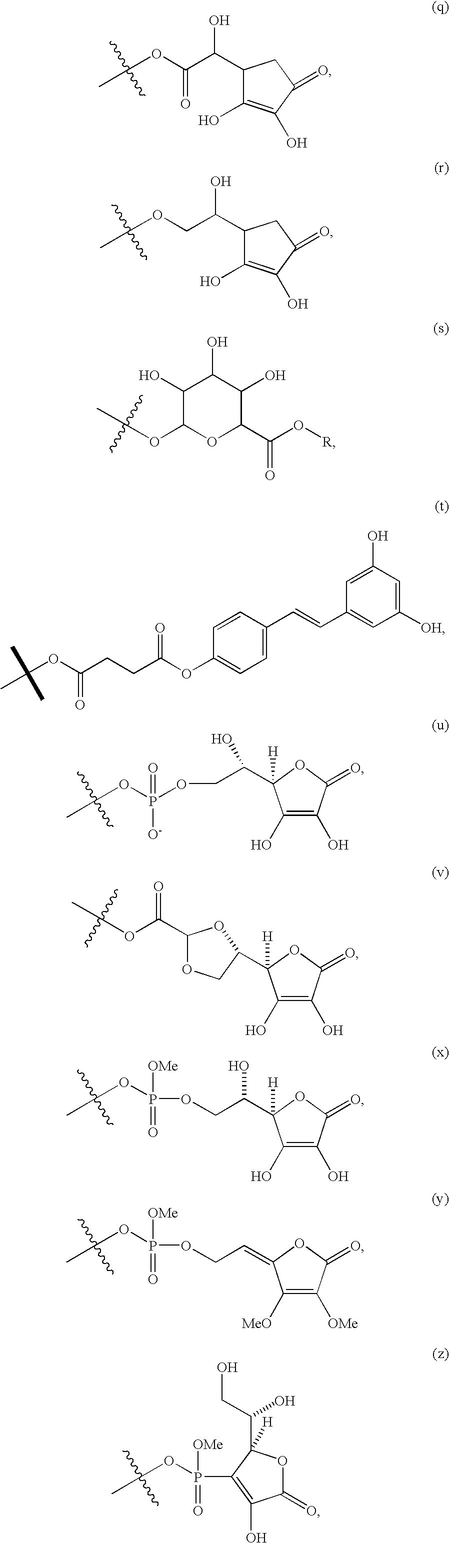 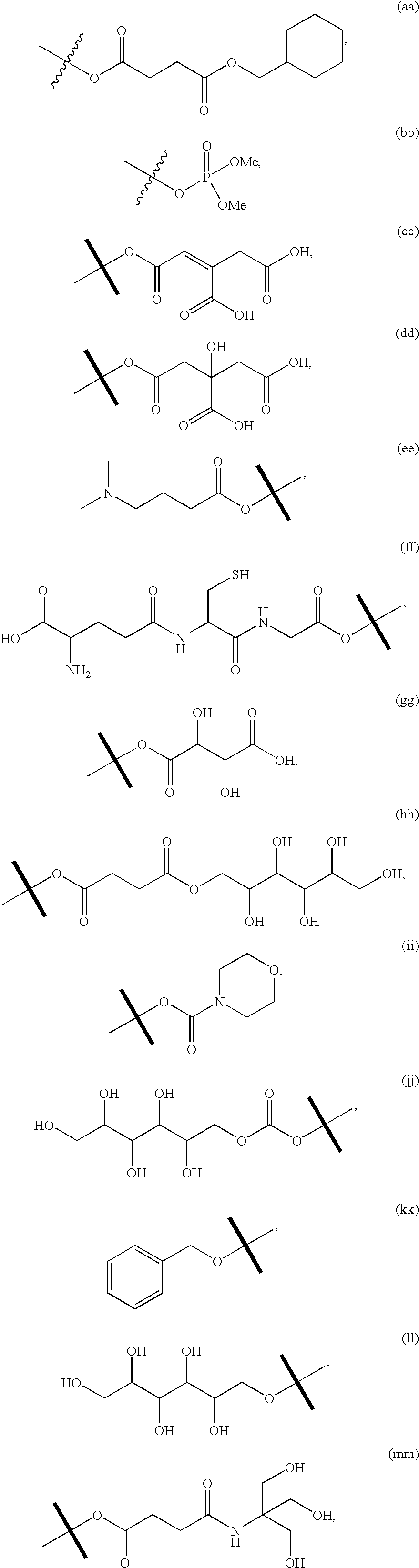 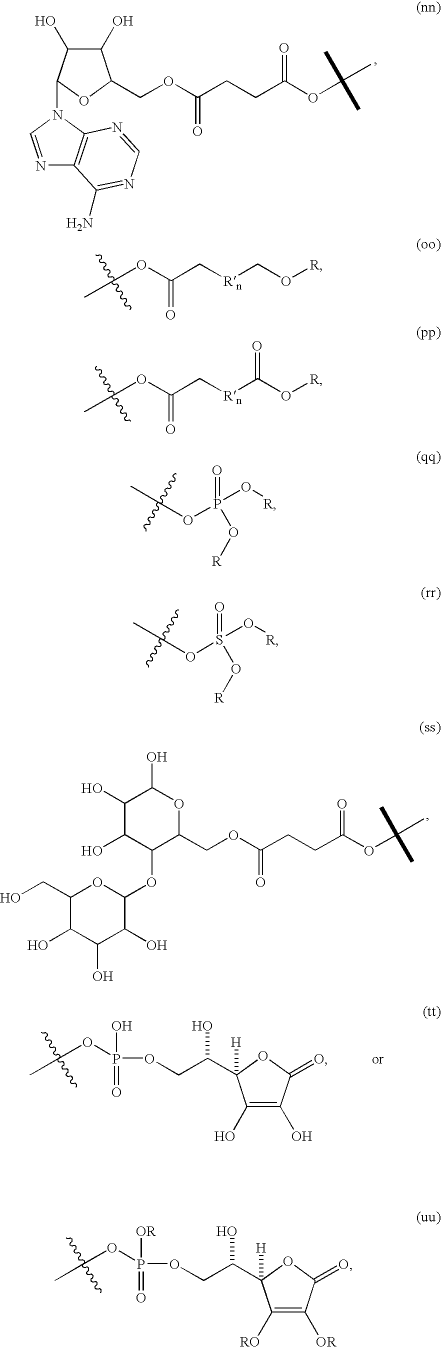
where each R is, for example, independently -alkyl-N R¹²₃⁺, -aromatic-N R¹²₃⁺, -alkyl-CO₂⁻, -aromatic-CO₂⁻, -amino acid-NH₃⁺, -phosphorylated amino acid-NH₃⁺, polyethylene glycol, dextran, H, alkyl, alkali metal, co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives), or aryl. Each R′ may be CH₂. n may range from 1 to 9. In some embodiments, substituents may include any combination of (d) through (uu). In some embodiments, negatively charged substituents may include alkali metals, one metal or a combination of different alkali metals in an embodiment with more than one negatively charged substituent, as counter ions. Alkali metals may include, but are not limited to, sodium, potassium, and/or lithium.

Water-soluble carotenoid analogs or derivatives may have a water solubility of greater than about 1 mg/mL in some embodiments. In certain embodiments, water-soluble carotenoid analogs or derivatives may have a water solubility of greater than about 5 mg/mL. In certain embodiments, water-soluble carotenoid analogs or derivatives may have a water solubility of greater than about 10 mg/mL. In some embodiments, water-soluble carotenoid analogs or derivatives may have a water solubility of greater than about 50 mg/mL.

Naturally occurring carotenoids such as xanthophyll carotenoids of the C40 series, which includes commercially important compounds such as lutein, zeaxanthin, and astaxanthin, have poor aqueous solubility in the native state. The aqueous solubility and/or dispersibility of derivatized carotenoids may be vastly increased by varying the chemical structure(s) of the esterified moieties.

In some embodiments, highly water-dispersible C40 carotenoid derivatives may include natural source RRR-lutein(β,ε-carotene-3,3′-diol) derivatives. Derivatives may be synthesized by esterification with inorganic phosphate and succinic acid, respectively, and subsequently converted to the sodium salts. Deep orange, evenly-colored aqueous suspensions were obtained after addition of these derivatives to USP-purified water. Aqueous dispersibility of the disuccinate sodium salt of natural lutein was 2.85 mg/mL; the dipshosphate salt demonstrated a >10-fold increase in dispersibility at 29.27 mg/mL. Aqueous suspensions may be obtained without the addition of heat, detergents, co-solvents, or other additives.

The direct aqueous superoxide scavenging abilities of these derivatives were subsequently evaluated by electron paramagnetic resonance (EPR) spectroscopy in a well-characterized in vitro isolated human neutrophil assay. The derivatives may be potent (millimolar concentration) and nearly identical aqueous-phase scavengers, demonstrating dose-dependent suppression of the superoxide anion signal (as detected by spin-trap adducts of DEPMPO) in the millimolar range. Evidence of card-pack aggregation was obtained for the disphosphate derivative with UV/V-Vis spectroscopy (discussed herein), whereas limited card-pack and/or head-to-tail aggregation was noted for the disuccinate derivative. These lutein-based soft drugs may find utility in those commercial and clinical applications for which aqueous-phase singlet oxygen quenching and direct radical scavenging may be required.

The absolute size of a carotenoid derivative (in 3 dimensions) is important when considering its use in biological and/or medicinal applications. Some of the largest naturally-occurring carotenoids are no greater than about C₅₀. This is probably due to size limits imposed on molecules requiring incorporation into and/or interaction with cellular membranes. Cellular membranes may be particularly co-evolved with molecules of a length of approximately 30 nm. In some embodiments, carotenoid derivatives may be greater than or less than about 30 nm in size. In certain embodiments, carotenoid derivatives may be able to change conformation and/or otherwise assume an appropriate shape which effectively enables the carotenoid derivative to efficiently interact with a cellular membrane.

Although the above structure, and subsequent structures, depict alkenes in the E configuration this should not be seen as limiting. Compounds discussed herein may include embodiments where alkenes are in the Z configuration or include alkenes in a combination of Z and E configurations within the same molecule. The compounds depicted herein may naturally convert between the Z and E configuration and/or exist in equilibrium between the two configurations.

In an embodiment, a chemical compound may include a carotenoid derivative having the structure (136) 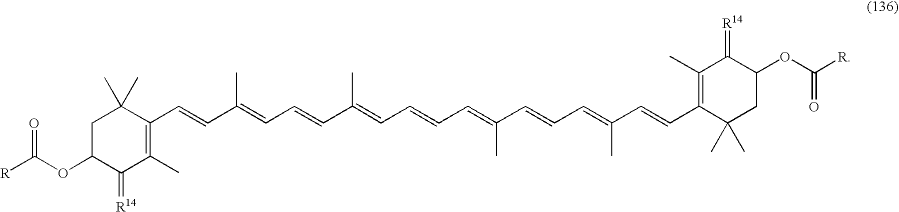
Each R¹⁴ may be independently O or H₂. Each R may be independently OR¹² or R¹². Each R¹² may be independently -alkyl-NR¹³₃⁺, -aromatic-NR¹³₃⁺, -alkyl-CO₂⁻, -aromatic-CO₂⁻, -amino acid-NH₃⁺, -phosphorylated amino acid-NH₃⁺, polyethylene glycol, dextran, H, alkyl, peptides, poly-lysine, co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives), or aryl. In addition, each R¹³ may be independently H, alkyl, or aryl. The carotenoid derivative may include at least one chiral center.

In a specific embodiment where R¹⁴ is H₂, the carotenoid derivative may have the structure (138)

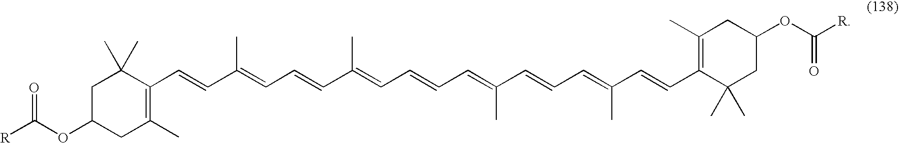

In a specific embodiment where R¹⁴ is O, the carotenoid derivative may have the structure (140)

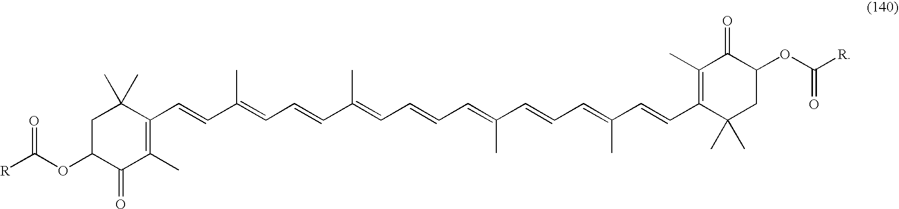

In an embodiment, a chemical compound may include a carotenoid derivative having the structure (142) 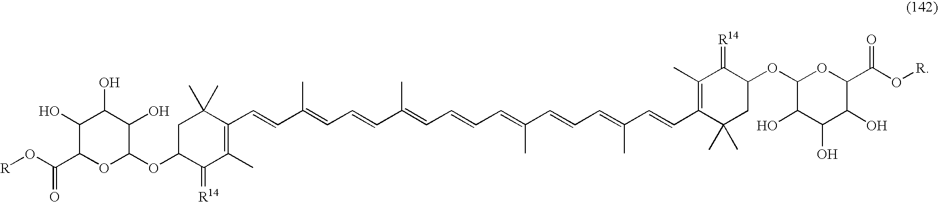
Each R¹⁴ may be independently O or H₂. Each R may be independently H, alkyl, benzyl, alkali metal, co-antioxidant, or aryl. The carotenoid derivative may include at least one chiral center. In a specific embodiment R¹⁴ may be H₂, the carotenoid derivative having the structure (144)

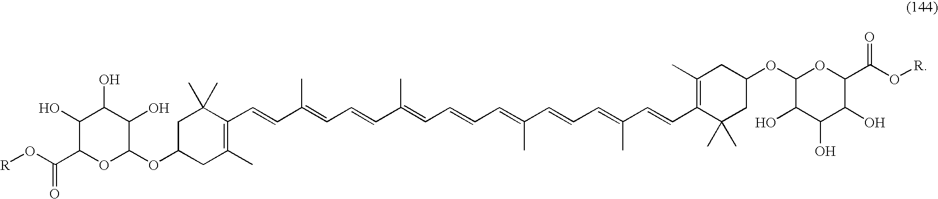

In a specific embodiment where R¹⁴ is O, the carotenoid derivative may have the structure (146)

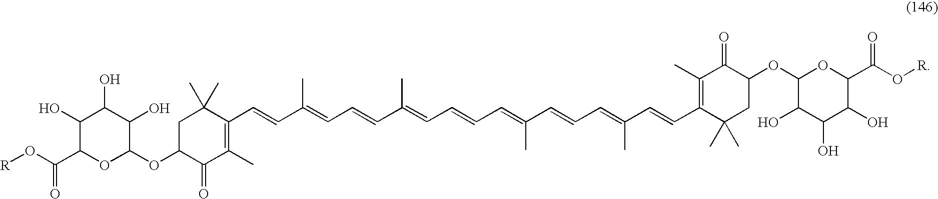

In an embodiment, a chemical compound may include a carotenoid derivative having the structure (148) 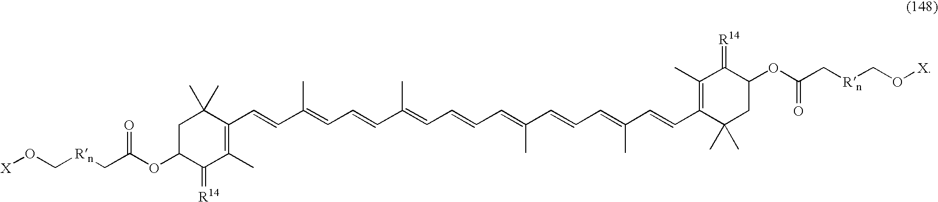
Each R¹⁴ may be independently O or H₂. Each R′ may be CH₂. n may range from 1 to 9. Each X may be independently 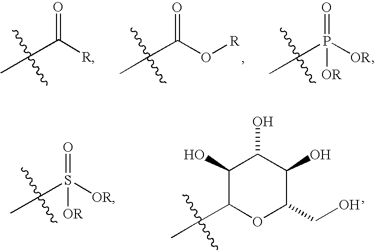
alkali metal, or co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives). Each R may be independently -alkyl-NR¹²₃⁺, -aromatic-NR¹²₃⁺, -alkyl-CO₂⁻, -aromatic-CO₂⁻, -amino acid-NH₃⁺, -phosphorylated amino acid-NH₃⁺, polyethylene glycol, dextran, H, alkyl, alkali metal, benzyl, co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives), or aryl. Each R¹² may be independently H, alkyl, or aryl. The carotenoid derivative may include at least one chiral center.

In a specific embodiment where R¹⁴ is H₂, the carotenoid derivative may have the structure (150) 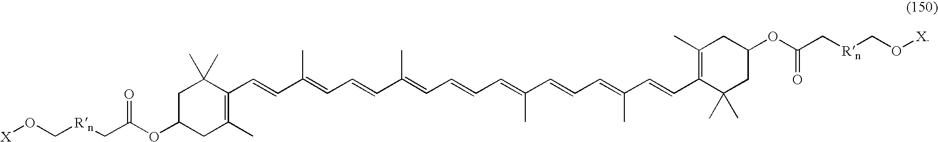
In a specific embodiment where R¹⁴ is O, the carotenoid derivative may have the structure (152)

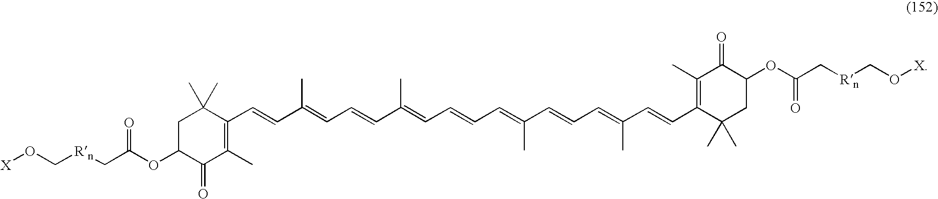

In an embodiment, a chemical compound may include a carotenoid derivative having the structure (148) 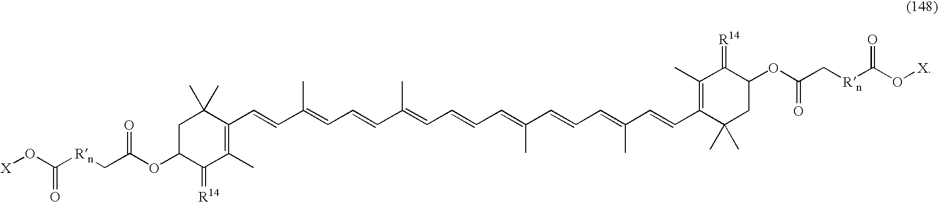
Each R¹⁴ may be independently O or H₂. Each R′ may be CH₂. n may range from 1 to 9. Each X may be independently 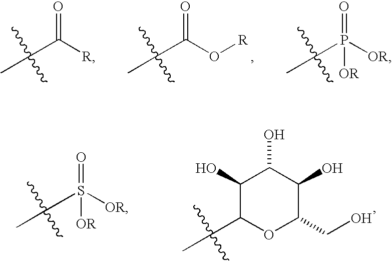
alkali metal, or co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives). Each R may be independently -alkyl-N R¹²₃⁺, -aromatic-N R¹²₃⁺, -alkyl-CO₂⁻, -aromatic-CO₂⁻, -amino acid-NH₃⁺, -phosphorylated amino acid-NH₃⁺, polyethylene glycol, dextran, H, alkyl, alkali metal, co-antioxidant (e.g. Vitamin C, Vitamin C analogs, Vitamin C derivatives, Vitamin E, Vitamin E analogs, Vitamin E derivatives, flavonoids, flavonoid analogs, or flavonoid derivatives), or aryl. Each R¹² may be independently H, alkyl or aryl. The carotenoid derivative may include at least one chiral center.

In a specific embodiment where R¹⁴ is H₂, the carotenoid derivative may have the structure (150) 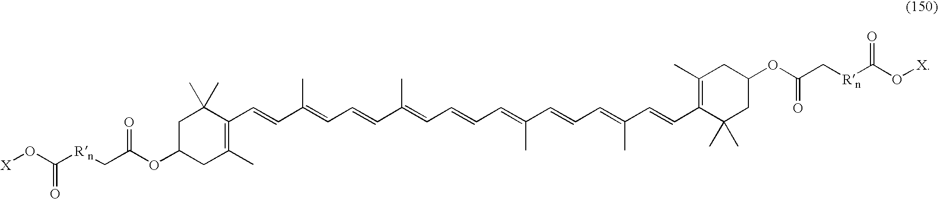
In a specific embodiment where R¹⁴ is O, the carotenoid derivative may have the structure (152)

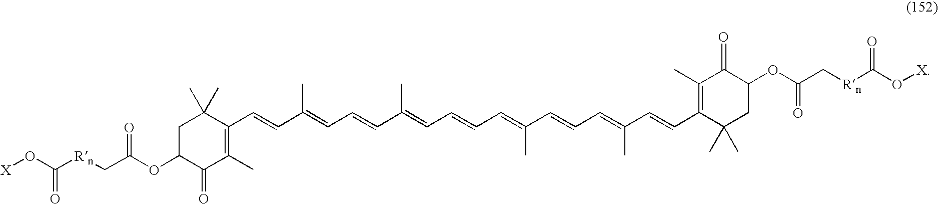

In an embodiment, a chemical compound may include a carotenoid derivative having the structure (154) 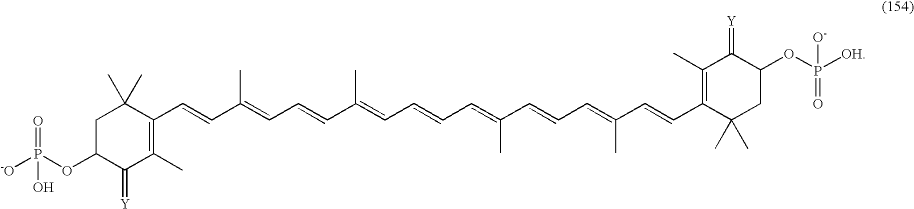
Each R¹⁴ may be independently O or H₂. The carotenoid derivative may include at least one chiral center. In a specific embodiment R¹⁴ may be H₂, the carotenoid derivative having the structure (156) 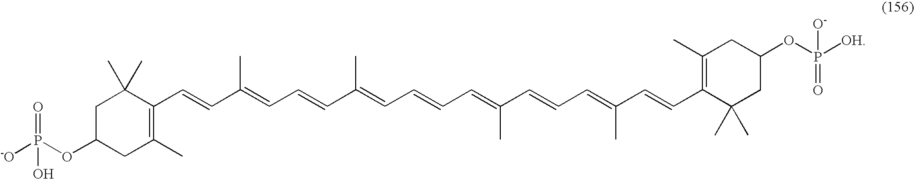
In a specific embodiment where R¹⁴ is O, the carotenoid derivative may have the structure (158)

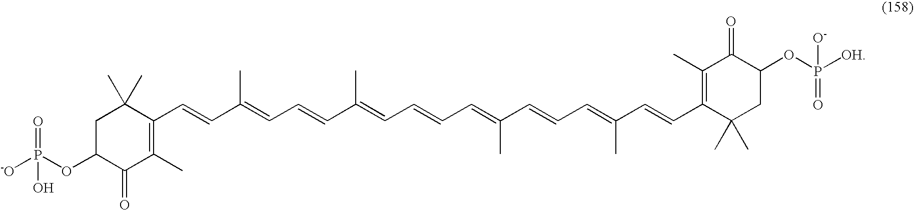

In some embodiments, a chemical compound may include a disuccinic acid ester carotenoid derivative having the structure (160)

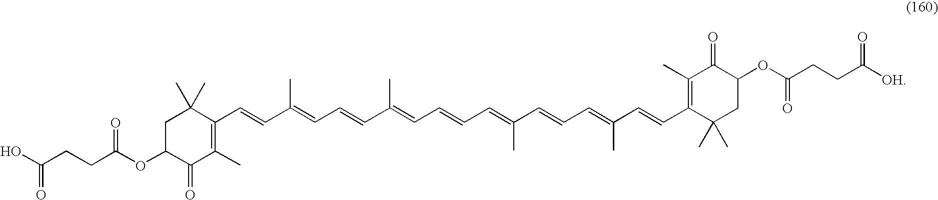

In some embodiments, a chemical compound may include a disodium salt disuccinic acid ester carotenoid derivative having the structure (162)

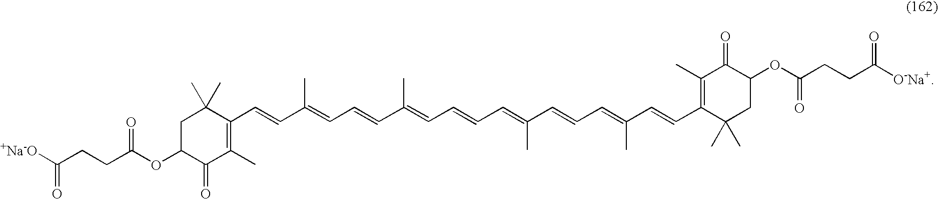

In some embodiments, a chemical compound may include a carotenoid derivative with a co-antioxidant, in particular one or more analogs or derivatives of vitamin C (i.e., L ascorbic acid) coupled to a carotenoid. Some 20 embodiments may include carboxylic acid and/or carboxylate derivatives of vitamin C coupled to a carotenoid (e.g., structure ( 164)) 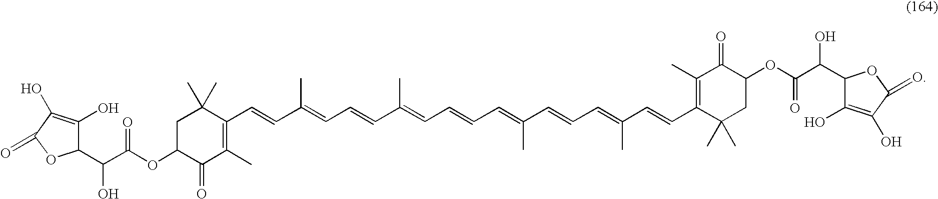
Carbohydr. Res. 1978, 60, 251-258, herein incorporated by reference, discloses oxidation at C-6 of ascorbic acid as depicted in EQN. 5. 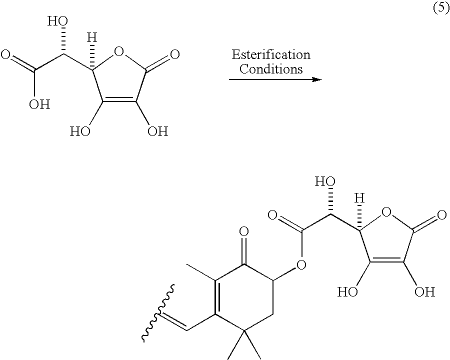
Some embodiments may include vitamin C and/or vitamin C analogs or derivatives coupled to a carotenoid. Vitamin C may be coupled to the carotenoid via an ether linkage (e.g., structure (166)) 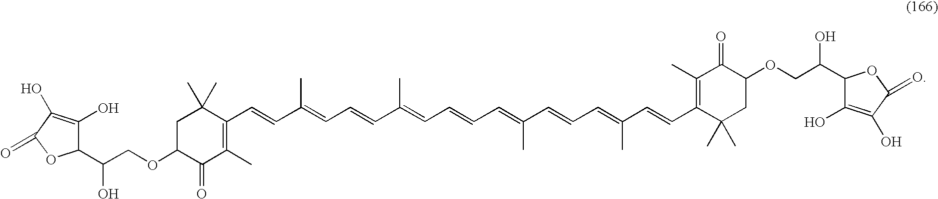
Some embodiments may include vitamin C disuccinate analogs or derivatives coupled to a carotenoid (e.g., structure (168)) 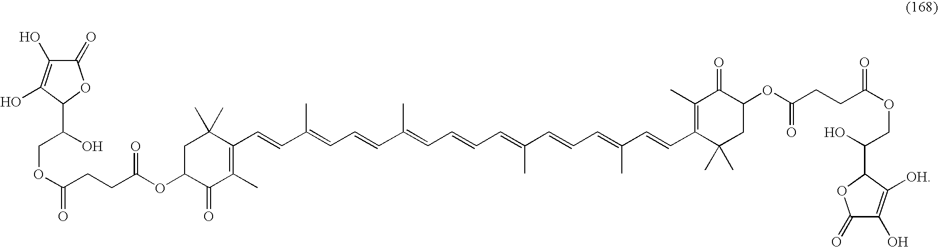
Some embodiments may include solutions or pharmaceutical preparations of carotenoids and/or carotenoid derivatives combined with co-antioxidants, in particular vitamin C and/or vitamin C analogs or derivatives. Pharmaceutical preparations may include about a 2:1 ratio of vitamin C to carotenoid respectively.

In some embodiments, co-antioxidants (e.g., vitamin C) may increase solubility of the chemical compound. In certain embodiments, co-antioxidants (e.g., vitamin C) may decrease toxicity associated with at least some carotenoid analogs or derivatives. In certain embodiments, co-antioxidants (e.g., vitamin C) may increase the potency of the chemical compound synergistically. Co-antioxidants may be coupled (e.g., a covalent bond) to the carotenoid derivative. Co-antioxidants may be included as a part of a pharmaceutically acceptable formulation.

In some embodiments, a carotenoid (e.g., astaxanthin) may be coupled to vitamin C forming an ether linkage. The ether linkage may be formed using the Mitsunobu reaction as in EQN. 1.

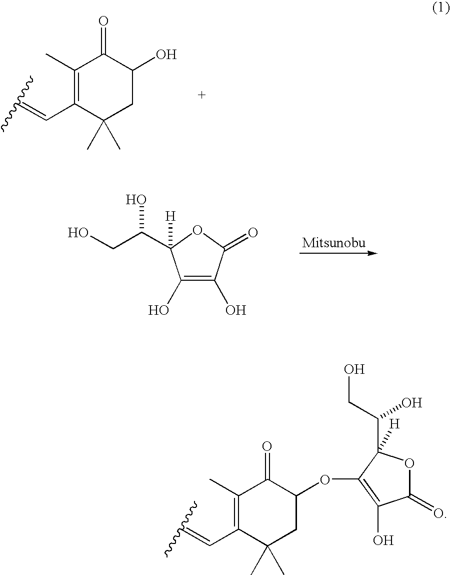

In some embodiments, vitamin C may be selectively esterified. Vitamin C may be selectively esterified at the C-3 position (e.g., EQN. 2). J. Org. Chem. 2000, 65, 911-913, herein incorporated by reference, discloses selective esterification at C-3 of unprotected ascorbic acid with primary alcohols.

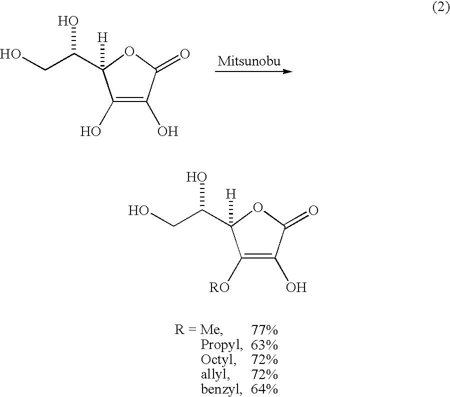

In some embodiments, a carotenoid may be coupled to vitamin C. Vitamin C may be coupled to the carotenoid at the C-6, C-5 diol position as depicted in EQNS. 3 and 4 forming an acetal.

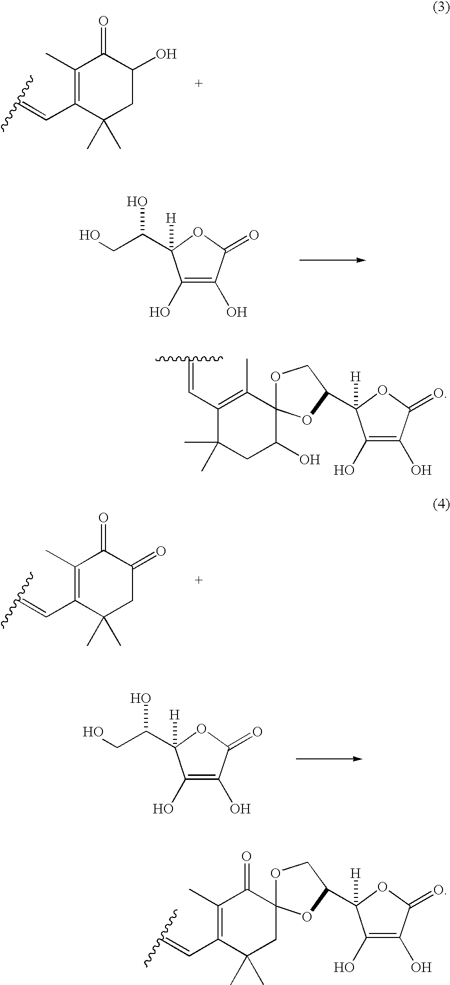

In some embodiments, a carotenoid may be coupled to a water-soluble moiety (e.g., vitamin C) with a glyoxylate linker as depicted in EQN. 6. Tetrahedron 1989, 22, 6987-6998, herein incorporated by reference, discloses similar acetal formations.

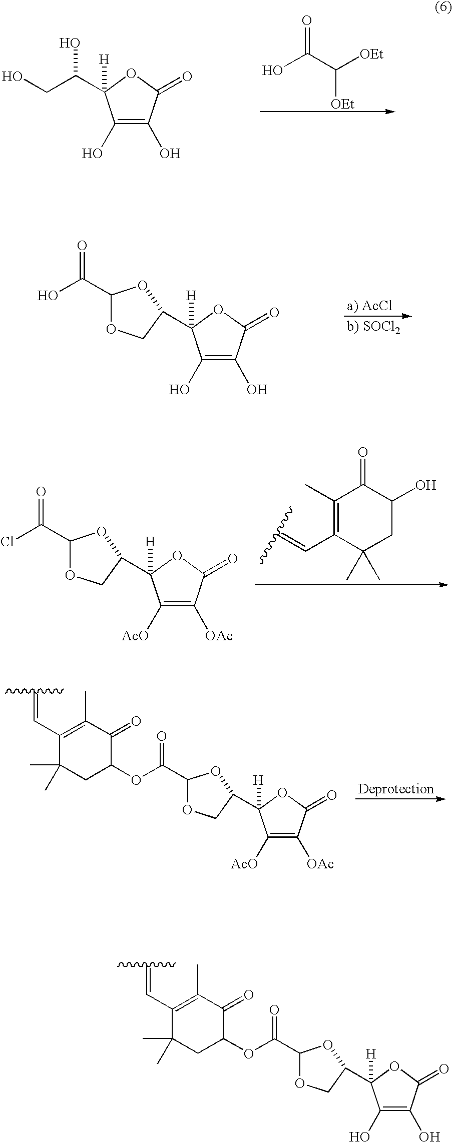

In some embodiments, a carotenoid may be coupled to a water-soluble moiety (e.g., vitamin C) with a glyoxylate linker as depicted in EQN. 7. J. Med. Chem. 1988, 31, 1363-1368, herein incorporated by reference, discloses the glyoxylic acid chloride.

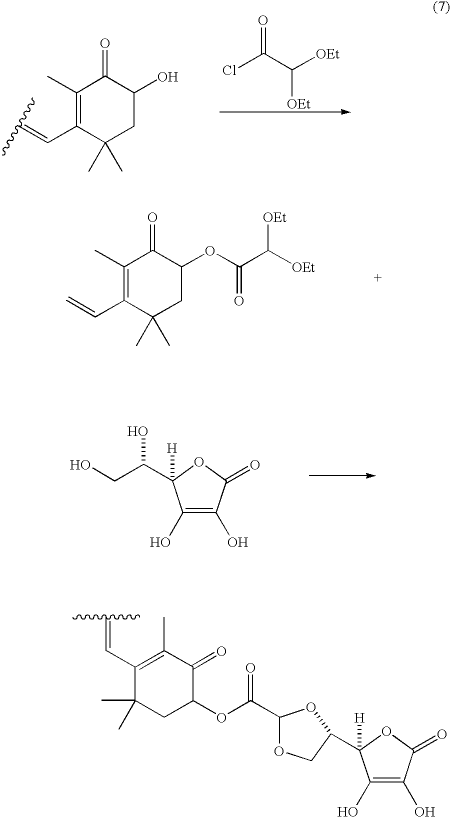

In some embodiments, a carotenoid may be coupled to a water-soluble moiety (e.g., vitamin C) with a phosphate linker as depicted in EQN. 8. Carbohydr. Res. 1988, 176, 73-78, herein incorporated by reference, discloses the L-ascorbate 6-phosphate.

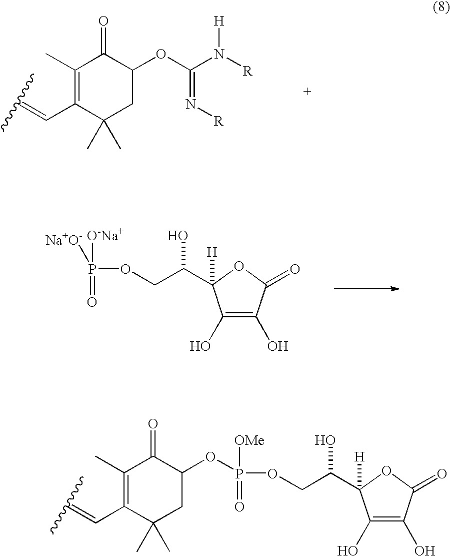

In some embodiments, a carotenoid may be coupled to a water-soluble moiety (e.g., vitamin C) with a phosphate linker as depicted in EQN. 9. Carbohydr. Res. 1979, 68, 313-319, herein incorporated by reference, discloses the 6-bromo derivative of vitamin C. Carbohydr. Res. 1988, 176, 73-78, herein incorporated by reference, discloses the 6-bromo derivative of vitamin C's reaction with phosphates.

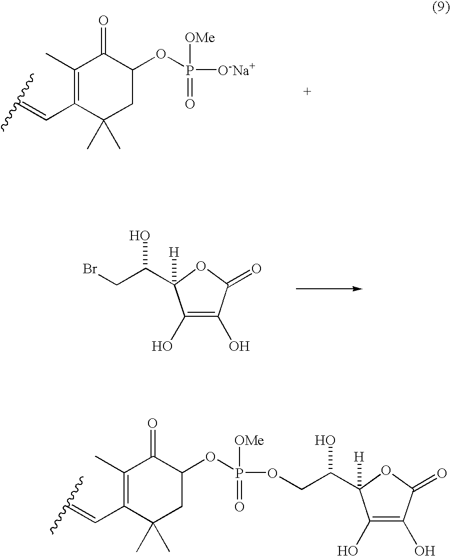

In some embodiments, a carotenoid may be coupled to a water-soluble moiety (e.g., vitamin C) with a phosphate linker as depicted in EQN. 10. J. Med Chem. 2001, 44, 1749-1757 and J Med Chem. 2001, 44, 3710-3720, herein incorporated by reference, disclose the allyl chloride derivative and its reaction with nucleophiles, including phosphates, under mild basic conditions.

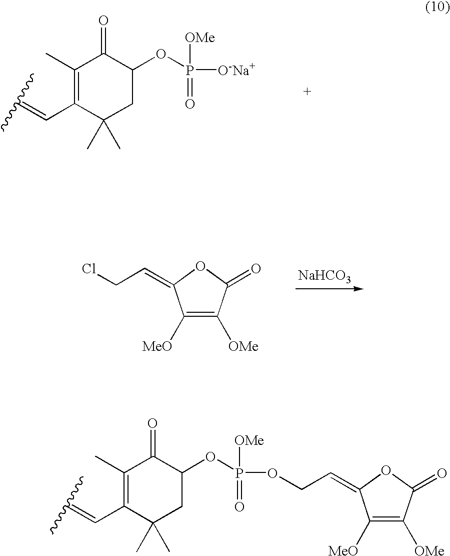

In some embodiments, a carotenoid may be coupled to a water-soluble moiety (e.g., vitamin C) with a phosphate linker as depicted in EQN. 11. Vitamin C may be coupled to the carotenoid using selective esterification at C-3 of unprotected ascorbic acid with primary alcohols.

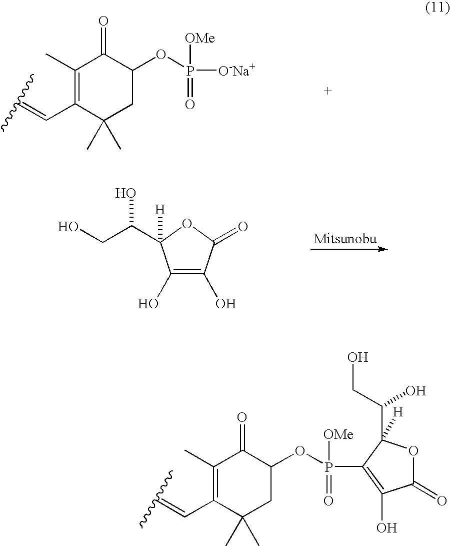

In some embodiments, a carotenoid may be coupled to a water-soluble moiety (e.g., vitamin C) with a phosphate linker as in 242. Structure 242 may include one or more counterions (e.g., alkali metals).

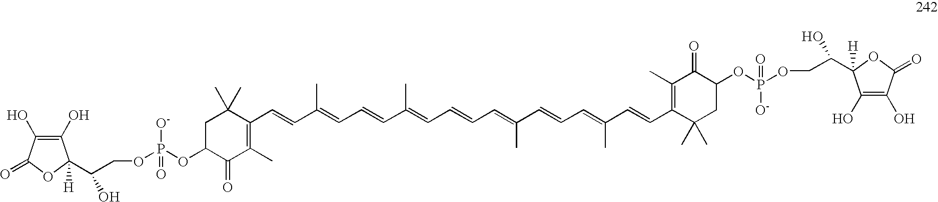

EQN. 12 depicts an example of a synthesis of a protected form of 242.

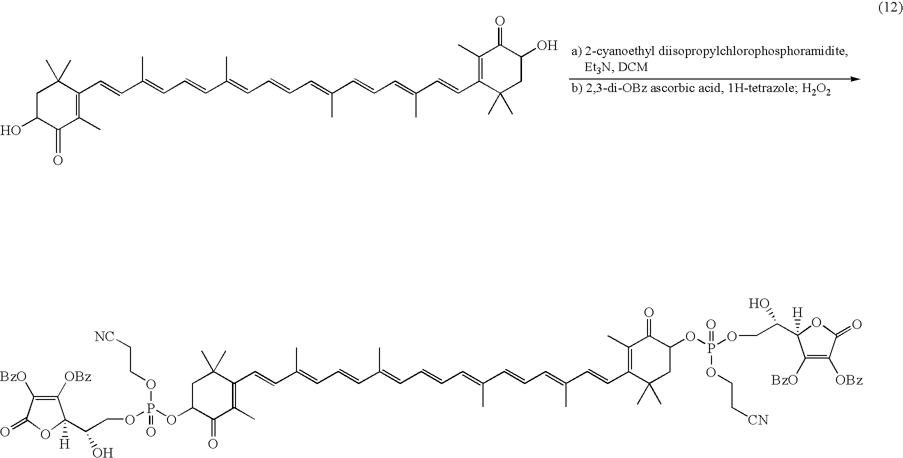

In some embodiments, a chemical compound may include a carotenoid derivative including one or more amino acids (e.g., lysine) and/or amino acid analogs or derivatives (e.g., lysine hydrochloric acid salt) coupled to a carotenoid (e.g., structure (170)).

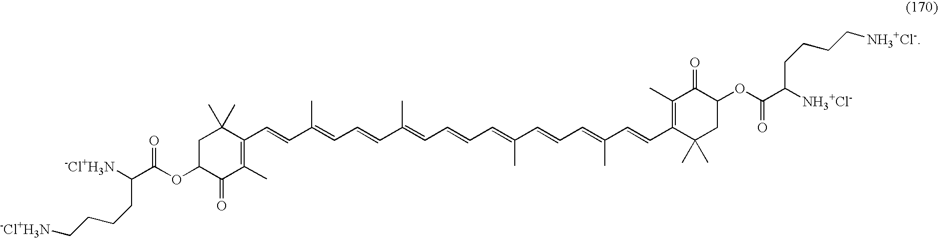

In some embodiments, a carotenoid analog or derivative may include:

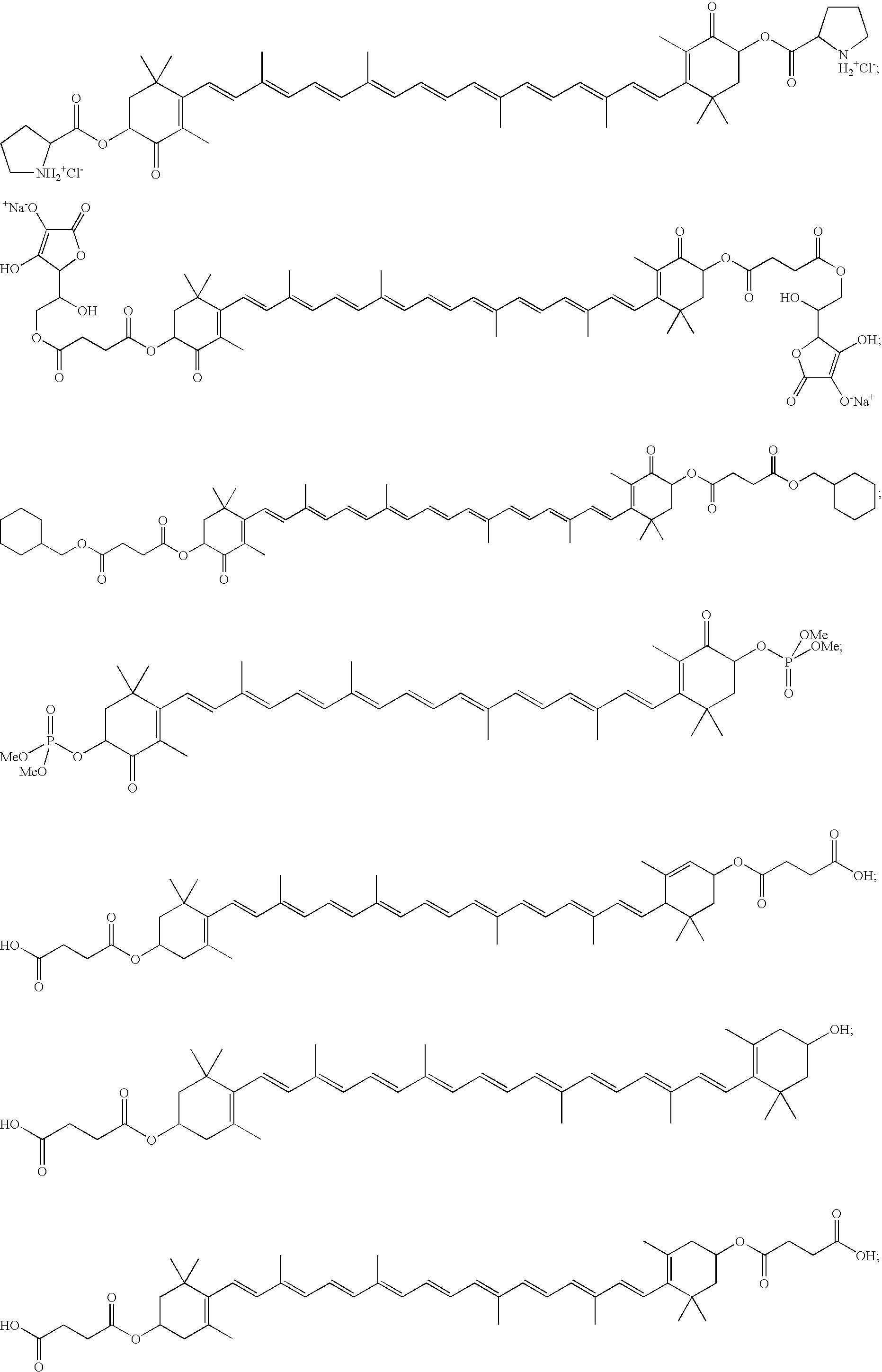

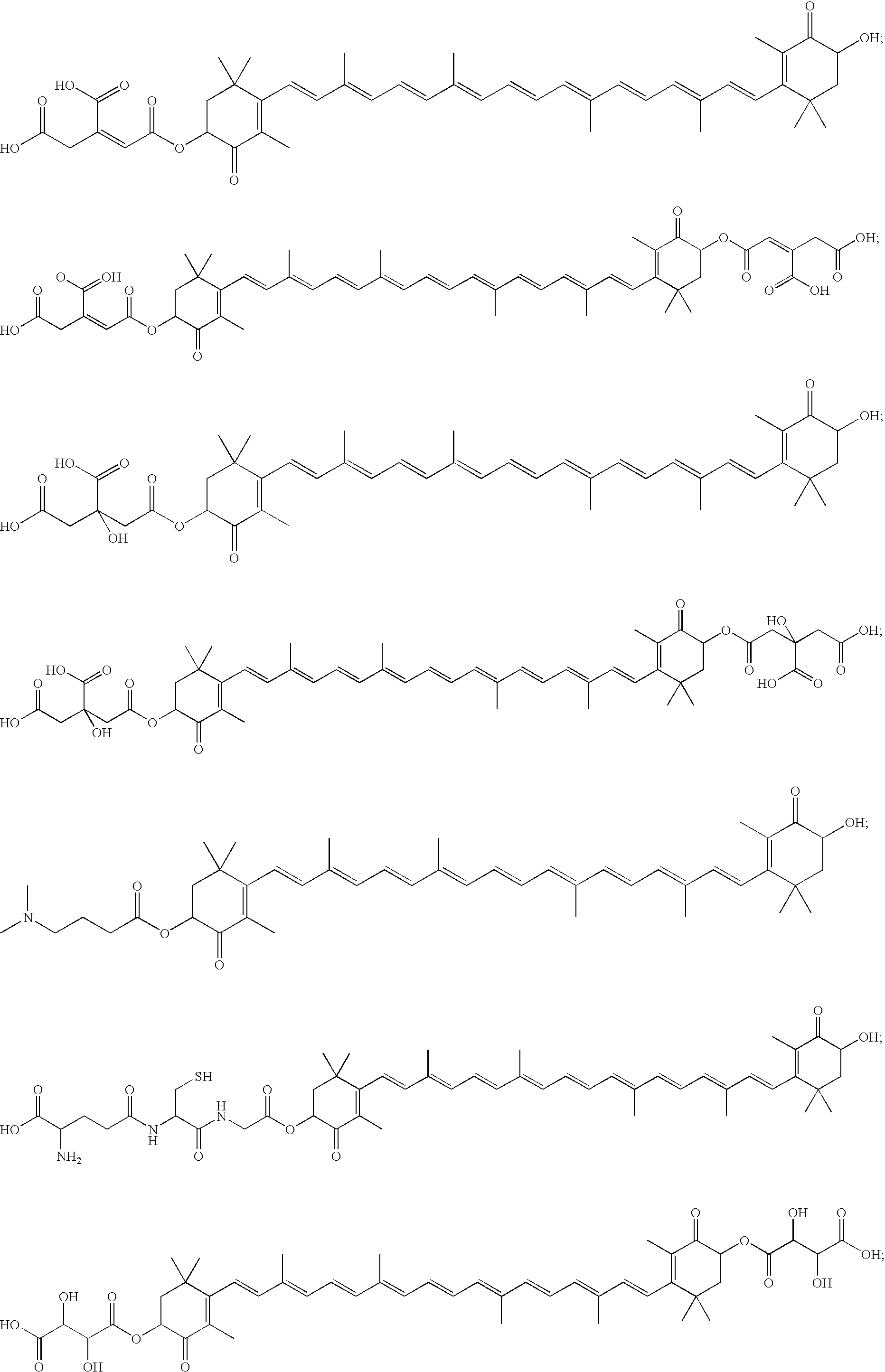

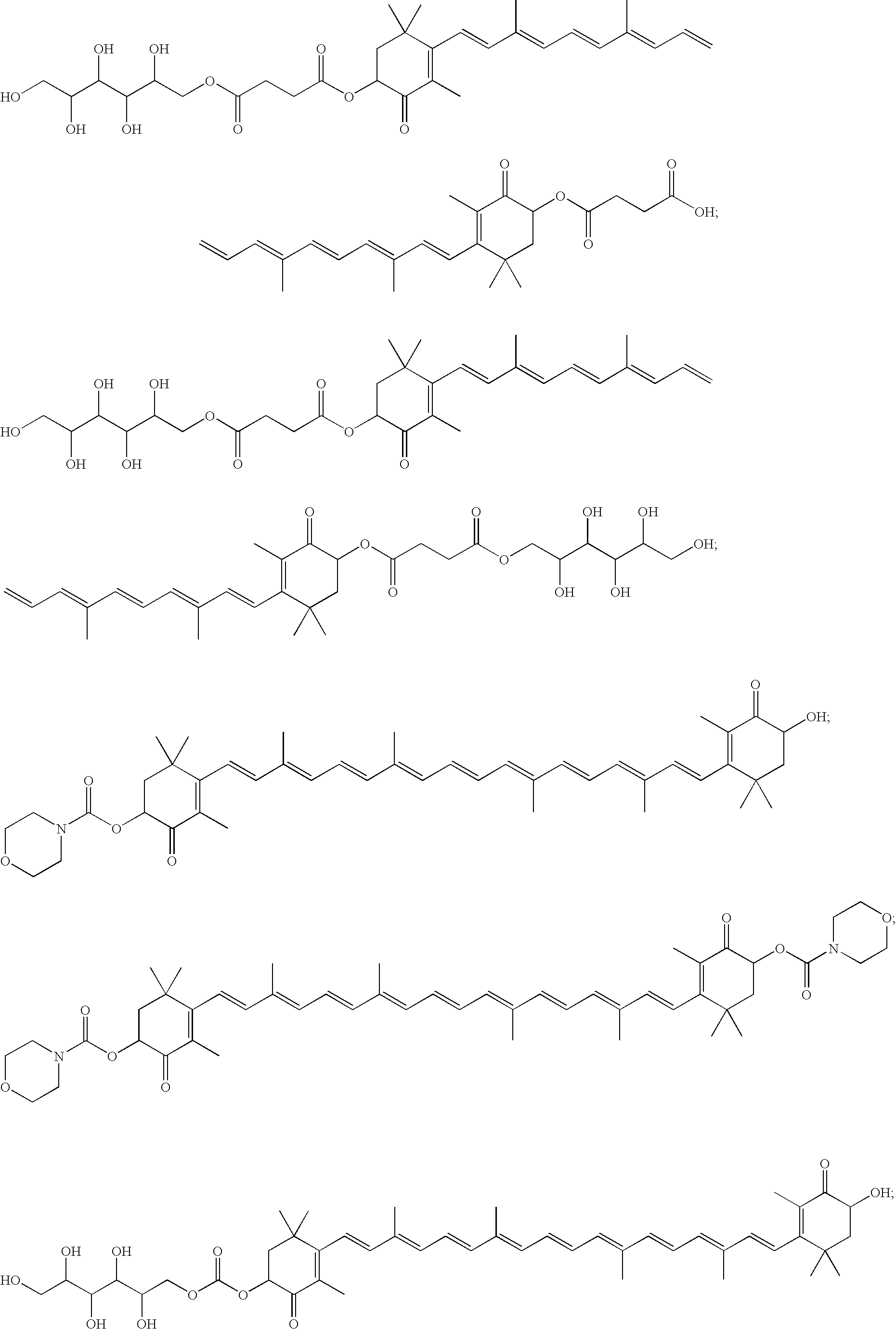

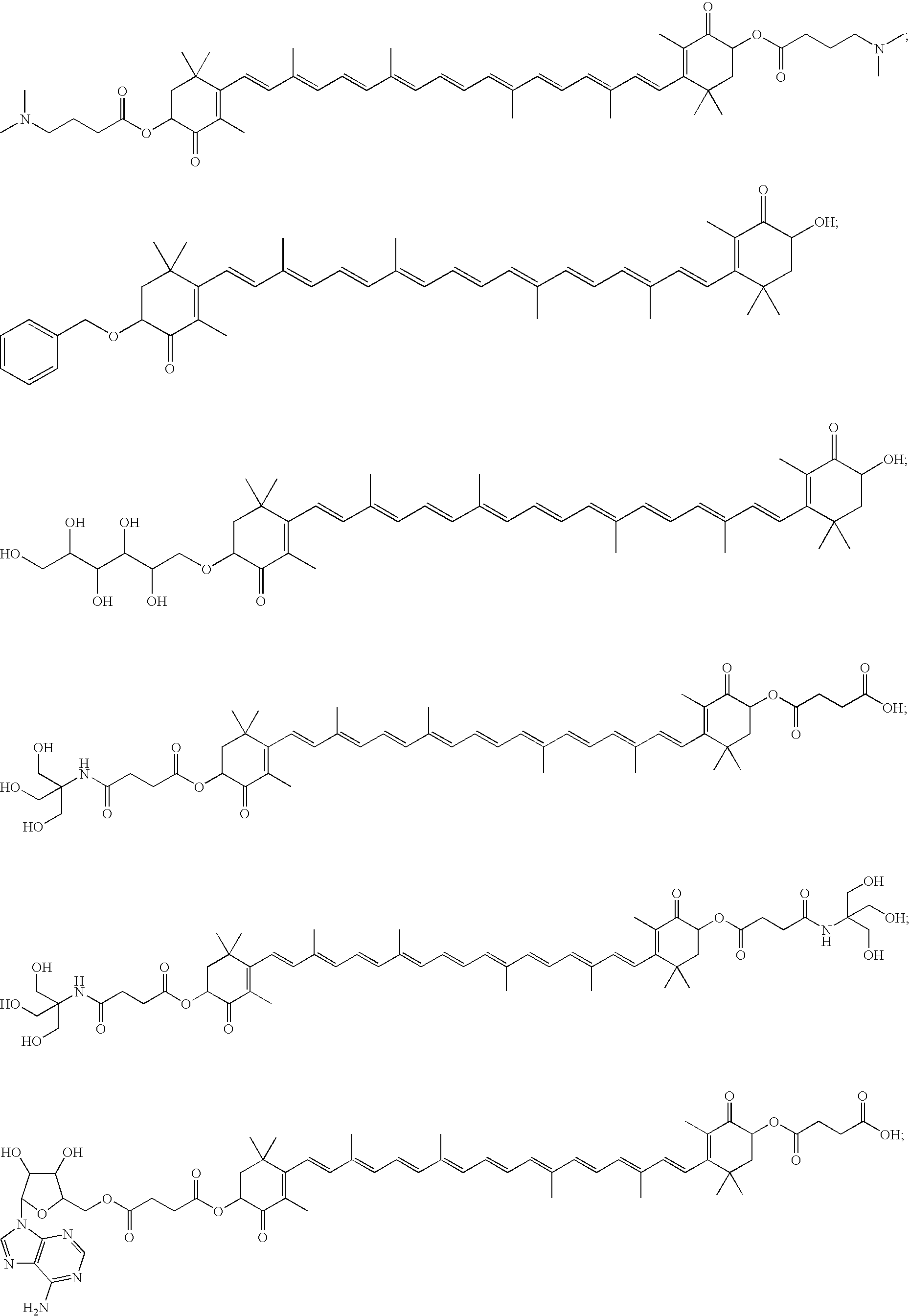

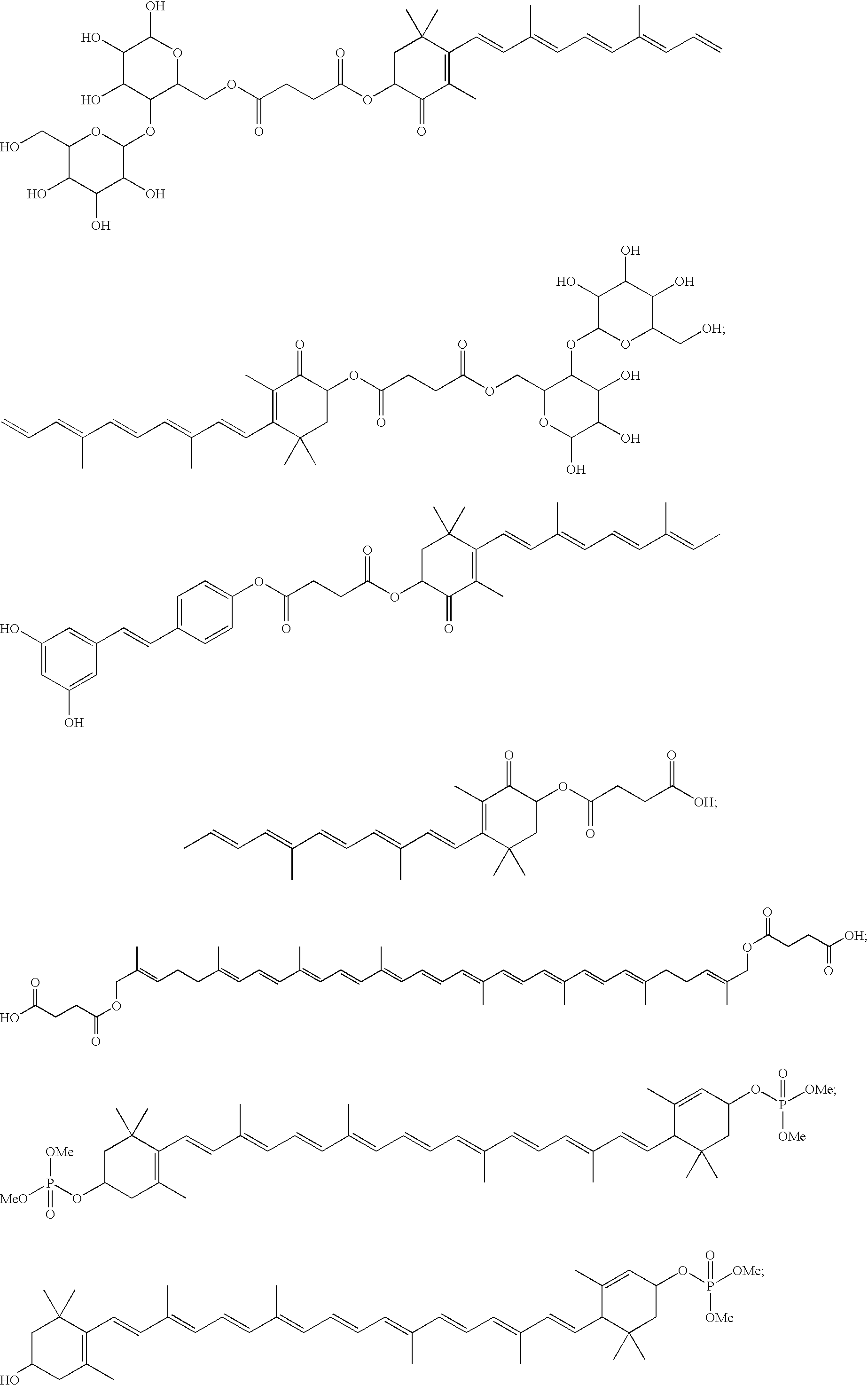

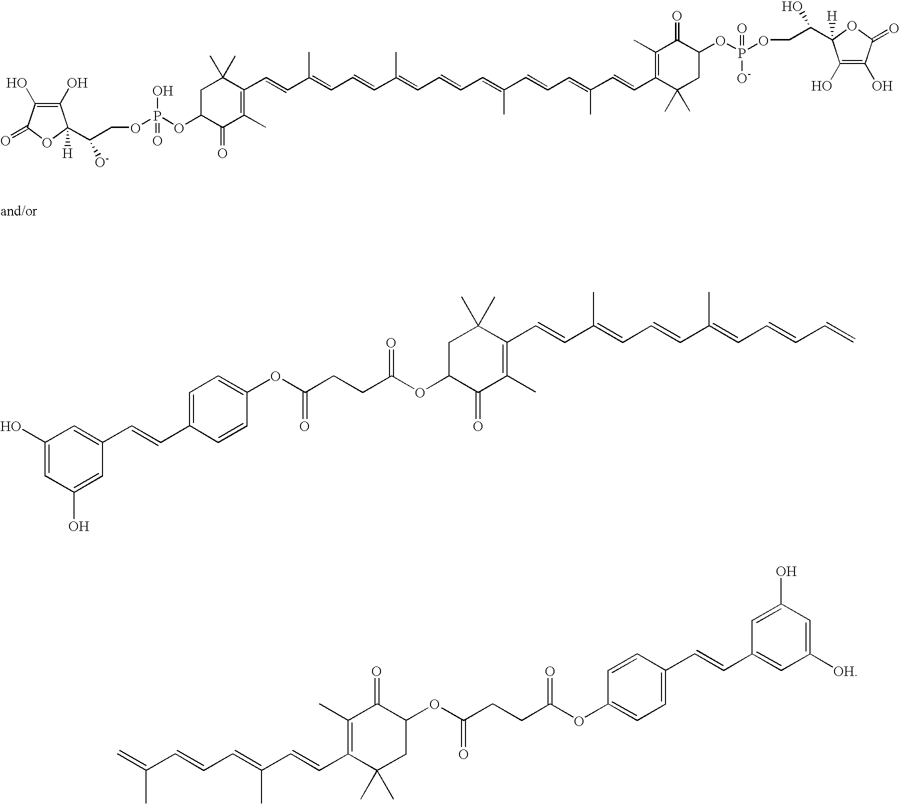

In some embodiments, a chemical compound may include a disuccinic acid ester carotenoid derivative having the structure ( 160)

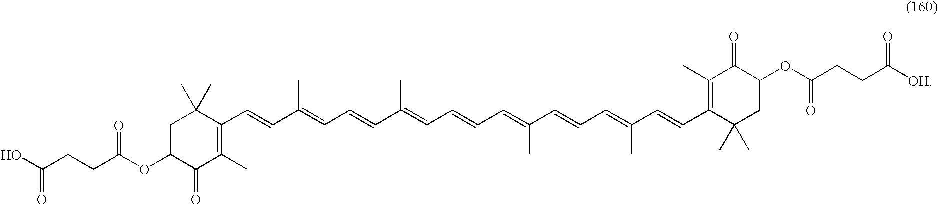

In some embodiments, a chemical compound may include a disodium salt disuccinic acid ester carotenoid derivative having the structure (162)

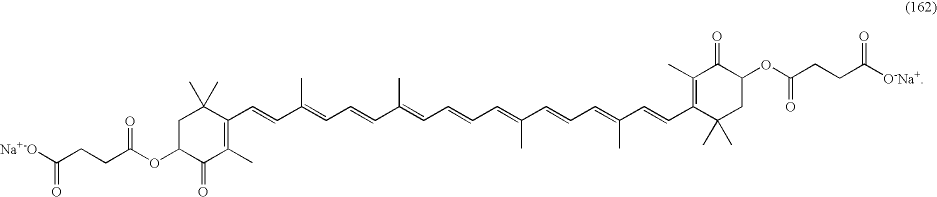

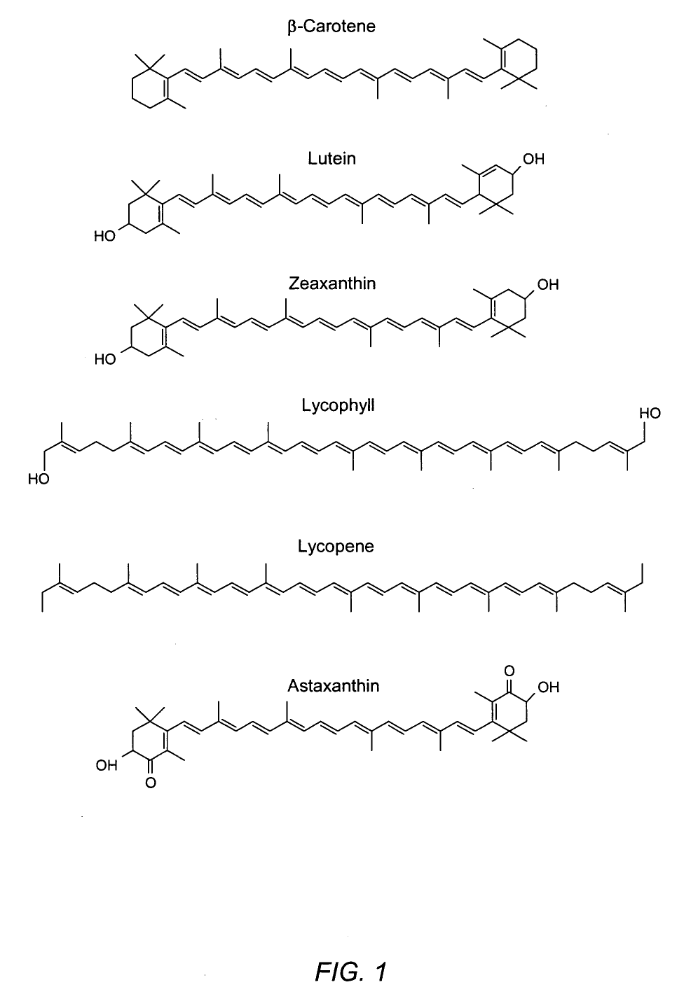

In an embodiment, the carotenoid derivatives may be synthesized from naturally-occurring carotenoids. The carotenoids may include structures 2A-2E depicted in FIG. 1. In some embodiments, the carotenoid derivatives may be synthesized from any naturally-occurring carotenoid including one or more alcohol substituents. In other embodiments, the carotenoid derivatives may be synthesized from a derivative of a naturally-occurring carotenoid including one or more alcohol substituents. The synthesis may result in a single stereoisomer. The synthesis may result in a single geometric isomer of the carotenoid derivative. The synthesis/synthetic sequence may include any prior purification or isolation steps carried out on the parent carotenoid.

In some embodiments, a synthesis may be a total synthesis using methods described herein to synthesize carotenoid derivatives and/or analogs. An example may include, but is not limited to, a 3S,3′S all-E carotenoid derivative, where the parent carotenoid is astaxanthin. The synthetic sequence may include protecting and subsequently deprotecting various functionalities of the carotenoid and/or substituent precursor. The alcohols may be deprotonated with a base. The deprotonated alcohol may be reacted with a substituent precursor with a good leaving group. The base may include any non-nucleophilic base known to one skilled in the art such as, for example, dimethylaminopyridine (DMAP). The deprotonated alcohol may act as a nucleophile reacting with the substituent precursor, displacing the leaving group. Leaving goups may include, but are not limited to, I, Cl, Br, tosyl, brosyl, mesyl, or trifyl. These are only a few examples of leaving groups that may be used, many more are known and would be apparent to one skilled in the art. In some embodiments, it may not even be necessary to deprotonate the alcohol, depending on the leaving group employed. In other examples the leaving group may be internal and may subsequently be included in the final structure of the carotenoid derivative, a non-limiting example may include anhydrides or strained cyclic ethers. For example, the deprotonated alcohol may be reacted with succinic anhydride. In an embodiment, the disuccinic acid ester of astaxanthin may be further converted to the disodium salt. Examples of synthetic sequences for the preparation of some of the specific embodiments depicted are described in the Examples section. The example depicted below is a generic non-limiting example of a synthetic sequence for the preparation of carotenoid derivatives.

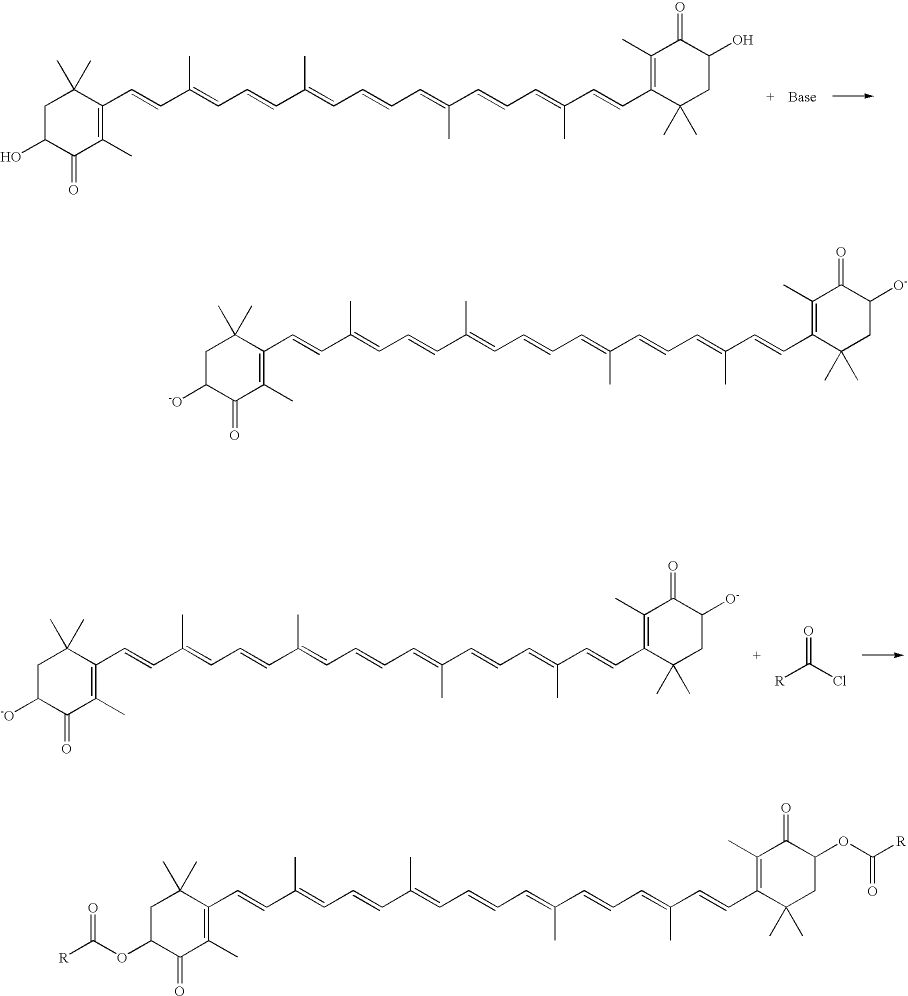

In some embodiments, the total synthesis of naturally-occurring as well as synthetic carotenoids as starting scaffolds for carotenoid analogs or derivatives may be a method of generation of said carotenoid analogs or derivatives.

In some embodiments, one or more of the conversions and/or reactions discussed herein may be carried out within one reaction vessel increasing the overall efficiency of the synthesis of the final product. In some embodiments, a product of one reaction during a total synthesis may not be fully worked up before continuing on with the following reaction. In general fully working up a reaction implies completely isolating and purify the product from a reaction. A reaction may instead only partially be worked up. For example, solid impurities which fall out of solution during the course of a reaction may be filtered off and the filtrate washed with solvent to ensure all of the resulting product is washed through and collected. In such a case the resulting collected product still in solution may not be isolated, but may then be combined with another reagent and further transformed. In some cases multiple transformations may be carried out in a single reaction flask simply by adding reagents one at a time without working up intermediate products. These types of “shortcuts” will improve the overall efficiency of a synthesis, especially when dealing with large scale reactions (e.g., along the lines of pilot plant scale and/or plant scale).

The synthetic preparation of carotenoid derivatives or analogs such as disodium disuccinate astaxanthin 162 at multigram scale (e.g., 200 g to 1 kg) is necessary if one wishes to produce these molecules commercially. Synthetic modifications of carotenoids, with the goal of increasing aqueous solubility and/or dispersibility, have been sparingly reported in the literature. At the time process development began, surveys of the peer-reviewed and patent literature indicated that neither a synthetic sequence nor an efficient process for the synthesis of 160 or 162 had been reported. Therefore, the bench-scale synthetic sequence and later the scale-up to multigram scale were optimized to improve both the yield and purity of the desired compound. Examples of synthetic preparation of carotenoids and carotenoid derivatives or analogs are illustrated in U.S. Patent Application Ser. No. 60/615,032 filed on Oct. 1, 2004, entitled “METHODS FOR SYNTHESIS OF CAROTENOIDS, INCLUDING ANALOGS, DERIVATIVES, AND SYNTHETIC AND BIOLOGICAL INTERMEDIATES” to Lockwood et al. which is incorporated by reference as if fully set forth herein.

The disodium disuccinate derivatives of synthetic astaxanthin were successfully synthesized in gram amounts and at high purity (>90%) area under the curve (AUC) by HPLC. The compound in “racemic” form demonstrated water “dispersibility” of 8.64 mg/mL, a significant improvement over the parent compound astaxanthin, which is insoluble in water. Initial biophysical characterization demonstrated that Cardax™ derivatives (as both the statistical mixture of stereoisomers and as individual stereoisomers) were potent direct scavengers of superoxide anion in the aqueous phase, the first such description in this model system for a C40 carotenoid. Plasma-protein binding studies in vitro revealed that the meso-(3R,3′S)-disodium disuccinate astaxanthin derivative bound immediately and preferentially to human serum albumin (HSA) at a binding site, suggesting that beneficial ligand-binding associations might take place in vivo after parenteral administration of the compound. The single- and multiple-dose pharmacokinetics of an oral preparation of the racemic compound (in lipophilic emulsion) were then investigated in a murine model, and significant plasma and tissue levels of nonesterified astaxanthin were achieved. Proof-of-concept studies in ischemia-reperfusion injury performed in rodents subsequently revealed that intravenous pretreatment with Cardax™ was significantly cardioprotective and achieved myocardial salvage in this experimental infarction model (e.g., up to 56% at the highest dose tested). The test material for three of the studies described above was obtained from a single pilot batch of compound (>200 g single batch at >97% purity by HPLC).

In some embodiments, it may be advantageous to be able to efficiently separate out individual stereoisomers of a racemic mixture of a chemical compound. Efficiently separating out individual stereoisomers on a relatively large scale may advantageously increase availability of starting materials.

In some embodiments, chromatographic separation techniques may be used to separate stereoisomers of a racemic mixture. In some embodiments pure optically active stereoisomers may be reacted with a mixture of stereoisomers of a chemical compound to form a mixture of diastereomers. Diastereomers may have different physical properties as opposed to stereoisomers, thus making it easier to separate diastereomers.

For example it may be advantageous to separate out stereoisomers from a racemic mixture of astaxanthin. In some embodiments, astaxanthin may be coupled to an optically active compound (e.g., dicamphanic acid). Coupling astaxanthin to optically active compounds produces diastereomers with different physical properties. The diastereomers produced may be separated using chromatographic separation techniques as described herein.

Bulk chromatographic separation of the diastereomeric dicamphanic acid ester(s) of synthetic astaxanthin at preparative chromatography scale was performed to subsequently make gram-scale quantities of each stereoisomer of disodium disuccinate ester astaxanthin.

As used herein the terms “structural carotenoid analogs or derivatives” may be generally defined as carotenoids and the biologically active structural analogs or derivatives thereof. “Derivative” in the context of this application is generally defined as a chemical substance derived from another substance either directly or by modification or partial substitution. “Analog” in the context of this application is generally defined as a compound that resembles another in structure but is not necessarily an isomer. Typical analogs or derivatives include molecules which demonstrate equivalent or improved biologically useful and relevant function, but which differ structurally from the parent compounds. Parent carotenoids are selected from the more than 700 naturally-occurring carotenoids described in the literature, and their stereo- and geometric isomers. Such analogs or derivatives may include, but are not limited to, esters, ethers, carbonates, amides, carbamates, phosphate esters and ethers, sulfates, glycoside ethers, with or without spacers (linkers).

As used herein the terms “the synergistic combination of more than one structural analog or derivative or synthetic intermediate of carotenoids” may be generally defined as any composition including one structural carotenoid analog or derivative or synthetic intermediate combined with one or more other structural carotenoid analogs or derivatives or synthetic intermediate or co-antioxidants, either as derivatives or in solutions and/or formulations.

In some embodiments, techniques described herein may be applied to the inhibition and/or amelioration of proiferative disorder, including but not limted to neoplastic transformation of one or more cells.

An embodiment may include the administration of structural carotenoid analogs or derivatives or synthetic intermediates alone or in combination to a subject such that the occurrence of a proliferative disorder is thereby inhibited and/or ameliorated. The structural carotenoid analogs or derivatives or synthetic intermediates may be water-soluble and/or water dispersible derivatives. The carotenoid derivatives may include any substituent that substantially increases the water solubility of the naturally-occurring carotenoid. The carotenoid derivatives may retain and/or improve the antioxidant properties of the parent carotenoid. The carotenoid derivatives may retain the non-toxic properties of the parent carotenoid. The carotenoid derivatives may have increased bioavailability, relative to the parent carotenoid, upon administration to a subject. The parent carotenoid may be naturally occurring.

Another embodiments may include the administration of a composition comprised of the synergistic combination of more than one structural analog or derivative or synthetic intermediate of carotenoids to a subject such that the occurrence of a proliferative disorder is thereby reduced. The composition may be a “racemic” (i.e. mixture of the potential stereoisomeric forms) mixture of carotenoid derivatives. Included as well are pharmaceutical compositions comprised of structural analogs or derivatives or synthetic intermediates of carotenoids in combination with a pharmaceutically acceptable carrier. In one embodiment, a pharmaceutically acceptable carrier may be serum albumin. In one embodiment, structural analogs or derivatives or synthetic intermediates of carotenoids may be complexed with human serum albumin (i.e., HSA) in a solvent. HSA may act as a pharmaceutically acceptable carrier.

In some embodiments, a single stereoisomer of a structural analog or derivative or synthetic intermediate of carotenoids may be administered to a human subject in order to ameliorate a pathological condition. Administering a single stereoisomer of a particular compound (e.g., as part of a pharmaceutical composition) to a human subject may be advantageous (e.g., increasing the potency of the pharmaceutical composition). Administering a single stereoisomer may be advantageous due to the fact that only one isomer of potentially many may be biologically active enough to have the desired effect.

In some embodiments, compounds described herein may be administered in the form of nutraceuticals. “Nutraceuticals” as used herein, generally refers to dietary supplements, foods, or medical foods that: 1. possess health benefits generally defined as reducing the risk of a disease or health condition, including the management of a disease or health condition or the improvement of health; and 2. are safe for human consumption in such quantity, and with such frequency, as required to realize such properties. Generally a nutraceutical is any substance that is a food or a part of a food and provides medical or health benefits, including the prevention and treatment of disease. Such products may range from isolated nutrients, dietary supplements and specific diets to genetically engineered designer foods, herbal products, and processed foods such as cereals, soups and beverages. It is important to note that this definition applies to all categories of food and parts of food, ranging from dietary supplements such as folic acid, used for the prevention of spina bifida, to chicken soup, taken to lessen the discomfort of the common cold. This definition also includes a bio-engineered designer vegetable food, rich in antioxidant ingredients, and a stimulant functional food or pharmafood. Within the context of the description herein where the composition, use and/or delivery of pharmaceuticals are described nutraceuticals may also be composed, used, and/or delivered in a similar manner where appropriate.

Dosage and Administration

The xanthophyll carotenoid, carotenoid derivative or analog may be administered at a dosage level up to conventional dosage levels for xanthophyll carotenoids, carotenoid derivatives or analogs, but will typically be less than about 2 gm per day. Suitable dosage levels may depend upon the overall systemic effect of the chosen xanthophyll carotenoids, carotenoid derivatives or analogs, but typically suitable levels will be about 0.001 to 50 mg/kg body weight of the patient per day, from about 0.005 to 30 mg/kg per day, or from about 0.05 to 10 mg/kg per day. The compound may be administered on a regimen of up to 6 times per day, between about 1 to 4 times per day, or once per day.

In the case where an oral composition is employed, a suitable dosage range is, e.g. from about 0.01 mg to about 100 mg of a xanthophyll carotenoid, carotenoid derivative or analog per kg of body weight per day, preferably from about 0.1 mg to about 10 mg per kg and for cytoprotective use from 0.1 mg to about 100 mg of a xanthophyll carotenoid, carotenoid derivative or analog per kg of body weight per day.

It will be understood that the dosage of the therapeutic agents will vary with the nature and the severity of the condition to be treated, and with the particular therapeutic agents chosen. The dosage will also vary according to the age, weight, physical condition and response of the individual patient. The selection of the appropriate dosage for the individual patient is within the skills of a clinician.

In some embodiments, compositions may include all compositions of 1.0 gram or less of a particular structural carotenoid analog, in combination with 1.0 gram or less of one or more other structural carotenoid analogs or derivatives or synthetic intermediates and/or co-antioxidants, in an amount which is effective to achieve its intended purpose. While individual subject needs vary, determination of optimal ranges of effective amounts of each component is with the skill of the art. Typically, a structural carotenoid analog or derivative or synthetic intermediates may be administered to mammals, in particular humans, orally at a dose of 5 to 100 mg per day referenced to the body weight of the mammal or human being treated for a particular disease. Typically, a structural carotenoid analog or derivative or synthetic intermediate may be administered to mammals, in particular humans, parenterally at a dose of between 5 to 1000 mg per day referenced to the body weight of the mammal or human being treated for a particular disease. In other embodiments, about 100 mg of a structural carotenoid analog or derivative or synthetic intermediate is either orally or parenterally administered to treat or prevent disease.

The unit oral dose may comprise from about 0.25 mg to about 1.0 gram, or about 5 to 25 mg, of a structural carotenoid analog. The unit parenteral dose may include from about 25 mg to 1.0 gram, or between 25 mg and 500 mg, of a structural carotenoid analog. The unit intracoronary dose may include from about 25 mg to 1.0 gram, or between 25 mg and 100 mg, of a structural carotenoid analog. The unit doses may be administered one or more times daily, on alternate days, in loading dose or bolus form, or titrated in a parenteral solution to commonly accepted or novel biochemical surrogate marker(s) or clinical endpoints as is with the skill of the art.

In addition to administering a structural carotenoid analog or derivative or synthetic intermediate as a raw chemical, the compounds may be administered as part of a pharmaceutical preparation containing suitable pharmaceutically acceptable carriers, preservatives, excipients and auxiliaries which facilitate processing of the structural carotenoid analog or derivative or synthetic intermediates which may be used pharmaceutically. The preparations, particularly those preparations which may be administered orally and which may be used for the preferred type of administration, such as tablets, softgels, lozenges, dragees, and capsules, and also preparations which may be administered rectally, such as suppositories, as well as suitable solutions for administration by injection or orally or by inhalation of aerosolized preparations, may be prepared in dose ranges that provide similar bioavailability as described above, together with the excipient. While individual needs may vary, determination of the optimal ranges of effective amounts of each component is within the skill of the art.

General guidance in determining effective dose ranges for pharmacologically active compounds and compositions for use in the presently described embodiments may be found, for example, in the publications of the International Conference on Harmonisation and in REMINGTON'S PHARMACEUTICAL SCIENCES, 8^{th} Edition Ed. Bertram G. Katzung, chapters 27 and 28, pp. 484-528 (Mack Publishing Company 1990) and yet further in BASIC & CLINICAL PHARMACOLOGY, chapters 5 and 66, (Lange Medical Books/McGraw-Hill, New York, 2001).

Pharmaceutical Compositions

Any suitable route of administration may be employed for providing a patient with an effective dosage of drugs of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. In certain embodiments, it may be advantageous that the compositions described herein be administered orally.

The compositions may include those compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the drugs used in the present invention are conveniently delivered in the form of an aerosol spray presentation from pressurized packs or nebulisers. The compounds may also be delivered as powders which may be formulated and the powder composition may be inhaled with the aid of an insufflation powder inhaler device.

Suitable topical formulations for use in the present embodiments may include transdermal devices, aerosols, creams, ointments, lotions, dusting powders, and the like.

In practical use, drugs used can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

The pharmaceutical preparations may be manufactured in a manner which is itself known to one skilled in the art, for example, by means of conventional mixing, granulating, dragee-making, softgel encapsulation, dissolving, extracting, or lyophilizing processes. Thus, pharmaceutical preparations for oral use may be obtained by combining the active compounds with solid and semi-solid excipients and suitable preservatives, and/or co-antioxidants. Optionally, the resulting mixture may be ground and processed. The resulting mixture of granules may be used, after adding suitable auxiliaries, if desired or necessary, to obtain tablets, softgels, lozenges, capsules, or dragee cores.

Suitable excipients may be fillers such as saccharides (e.g., lactose, sucrose, or mannose), sugar alcohols (e.g., mannitol or sorbitol), cellulose preparations and/or calcium phosphates (e.g., tricalcium phosphate or calcium hydrogen phosphate). In addition binders may be used such as starch paste (e.g., maize or corn starch, wheat starch, rice starch, potato starch, gelatin, tragacanth, methyl cellulose, hydroxypropylmethylcellulose, sodium carboxymethylcellulose, and/or polyvinyl pyrrolidone). Disintegrating agents may be added (e.g., the above-mentioned starches) as well as carboxymethyl-starch, cross-linked polyvinyl pyrrolidone, agar, or alginic acid or a salt thereof (e.g., sodium alginate). Auxiliaries are, above all, flow-regulating agents and lubricants (e.g., silica, talc, stearic acid or salts thereof, such as magnesium stearate or calcium stearate, and/or polyethylene glycol, or PEG). Dragee cores are provided with suitable coatings, which, if desired, are resistant to gastric juices. Softgelatin capsules (“softgels”) are provided with suitable coatings, which, typically, contain gelatin and/or suitable edible dye(s). Animal component-free and kosher gelatin capsules may be particularly suitable for the embodiments described herein for wide availability of usage and consumption. For this purpose, concentrated saccharide solutions may be used, which may optionally contain gum arabic, talc, polyvinyl pyrrolidone, polyethylene glycol (PEG) and/or titanium dioxide, lacquer solutions and suitable organic solvents or solvent mixtures, including dimethylsulfoxide (DMSO), tetrahydrofuran (THF), acetone, ethanol, or other suitable solvents and co-solvents. In order to produce coatings resistant to gastric juices, solutions of suitable cellulose preparations such as acetylcellulose phthalate or hydroxypropylmethyl-cellulose phthalate, may be used. Dye stuffs or pigments may be added to the tablets or dragee coatings or softgelatin capsules, for example, for identification or in order to characterize combinations of active compound doses, or to disguise the capsule contents for usage in clinical or other studies.

Other pharmaceutical preparations that may be used orally include push-fit capsules made of gelatin, as well as soft, thermally sealed capsules made of gelatin and a plasticizer such as glycerol or sorbitol. The push-fit capsules may contain the active compounds in the form of granules that may be mixed with fillers such as, for example, lactose, binders such as starches, and/or lubricants such as talc or magnesium stearate and, optionally, stabilizers and/or preservatives. In soft capsules, the active compounds may be dissolved or suspended in suitable liquids, such as fatty oils such as rice bran oil or peanut oil or palm oil, or liquid paraffin. In some embodiments, stabilizers and preservatives may be added.

In some embodiments, pulmonary administration of a pharmaceutical preparation may be desirable. Pulmonary administration may include, for example, inhalation of aerosolized or nebulized liquid or solid particles of the pharmaceutically active component dispersed in and surrounded by a gas.

Possible pharmaceutical preparations, which may be used rectally, include, for example, suppositories, which consist of a combination of the active compounds with a suppository base. Suitable suppository bases are, for example, natural or synthetic triglycerides, or paraffin hydrocarbons. In addition, it is also possible to use gelatin rectal capsules that consist of a combination of the active compounds with a base. Possible base materials include, for example, liquid triglycerides, polyethylene glycols, or paraffin hydrocarbons.

Suitable formulations for parenteral administration include, but are not limited to, aqueous solutions of the active compounds in water-soluble and/or water dispersible form, for example, water-soluble salts, esters, carbonates, phosphate esters or ethers, sulfates, glycoside ethers, together with spacers and/or linkers. Suspensions of the active compounds as appropriate oily injection suspensions may be administered, particularly suitable for intramuscular injection. Suitable lipophilic solvents, co-solvents (such as DMSO or ethanol), and/or vehicles including fatty oils, for example, rice bran oil or peanut oil and/or palm oil, or synthetic fatty acid esters, for example, ethyl oleate or triglycerides, may be used. Aqueous injection suspensions may contain substances that increase the viscosity of the suspension including, for example, sodium carboxymethyl cellulose, sorbitol, dextran, and/or cyclodextrins. Cyclodextrins (e.g., β-cyclodextrin) may be used specifically to increase the water solubility for parenteral injection of the structural carotenoid analog. Liposomal formulations, in which mixtures of the structural carotenoid analog or derivative with, for example, egg yolk phosphotidylcholine (E-PC), may be made for injection. Optionally, the suspension may contain stabilizers, for example, antioxidants such as BHT, and/or preservatives, such as benzyl alcohol.

The compounds of this invention can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They can be administered alone, but generally will be administered with a pharmaceutical carrier selected on the basis of the chosen route of administration and standard pharmaceutical practice.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired.

By way of general guidance, the daily oral dosage of each active ingredient, when used for the indicated effects, will range between about 0.001 to 1000 mg/kg of body weight, between about 0.01 to 100 mg/kg of body weight per day, or between about 1.0 to 20 mg/kg/day. Intravenously administered doses may range from about 1 to about 10 mg/kg/minute during a constant rate infusion. Compounds of this invention may be administered in a single daily dose, or the total daily dosage may be administered in divided doses of two, three, or four or more times daily.

The pharmaceutical compositions described herein may further be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compounds are typically administered in admixture with suitable pharmaceutical diluents, excipients, or carriers (collectively referred to herein as “pharmacologically inert carriers”) suitably selected with respect to the intended form of administration, that is, oral tablets, capsules, elixirs, syrups and the like, and consistent with conventional pharmaceutical practices.

For instance, for oral administration in the form of a tablet or capsule, the pharmacologically active component may be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Dosage forms (pharmaceutical compositions) suitable for administration may contain from about 1 milligram to about 100 milligrams or more of active ingredient per dosage unit. In these pharmaceutical compositions the active ingredient will ordinarily be present in an amount of about 0.5-95% by weight based on the total weight of the composition.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington's Pharmaceutical Sciences, Mack Publishing Company, a standard reference text in this field.

In some embodiments, compositions may include all compositions of 1.0 gram or less of a particular structural carotenoid analog, in combination with 1.0 gram or less of one or more other structural carotenoid analogs or derivatives or synthetic intermediates and/or co-antioxidants, in an amount which is effective to achieve its intended purpose. While individual subject needs vary, determination of optimal ranges of effective amounts of each component is with the skill of the art. Typically, a structural carotenoid analog or derivative or synthetic intermediates may be administered to mammals, in particular humans, orally at a dose of 5 to 100 mg per day referenced to the body weight of the mammal or human being treated for a particular disease. Typically, a structural carotenoid analog or derivative or synthetic intermediate may be administered to mammals, in particular humans, parenterally at a dose of between 5 to 1000 mg per day referenced to the body weight of the mammal or human being treated for a particular disease. In other embodiments, about 100 mg of a structural carotenoid analog or derivative or synthetic intermediate is either orally or parenterally administered to treat or prevent disease.

The unit oral dose may comprise from about 0.25 mg to about 1.0 gram, or about 5 to 25 mg, of a structural carotenoid analog. The unit parenteral dose may include from about 25 mg to 1.0 gram, or between 25 mg and 500 mg, of a structural carotenoid analog. The unit intracoronary dose may include from about 25 mg to 1.0 gram, or between 25 mg and 100 mg, of a structural carotenoid analog. The unit doses may be administered one or more times daily, on alternate days, in loading dose or bolus form, or titrated in a parenteral solution to commonly accepted or novel biochemical surrogate marker(s) or clinical endpoints as is with the skill of the art.

In addition to administering a structural carotenoid analog or derivative or synthetic intermediate as a raw chemical, the compounds may be administered as part of a pharmaceutical preparation containing suitable pharmaceutically acceptable carriers, preservatives, excipients and auxiliaries which facilitate processing of the structural carotenoid analog or derivative or synthetic intermediates which may be used pharmaceutically. The preparations, particularly those preparations which may be administered orally and which may be used for the preferred type of administration, such as tablets, softgels, lozenges, dragees, and capsules, and also preparations which may be administered rectally, such as suppositories, as well as suitable solutions for administration by injection or orally or by inhalation of aerosolized preparations, may be prepared in dose ranges that provide similar bioavailability as described above, together with the excipient. While individual needs may vary, determination of the optimal ranges of effective amounts of each component is within the skill of the art.

### EXAMPLES

Having now described the invention, the same will be more readily understood through reference to the following example(s), which are provided by way of illustration, and are not intended to be limiting of the present invention. General. Natural source lutein (90%) was obtained from ChemPacific, Inc. (Baltimore, Md.) as a red-orange solid and was used without further purification. All other reagents and solvents used were purchased from Acros (New Jersey, USA) and were used without further purification. All reactions were performed under N₂ atmosphere. All flash chromatographic purifications were performed on Natland International Corporation 230-400 mesh silica gel using the indicated solvents. LC/MS (APCI) and LC/MS (ESI) were recorded on an Agilent 1100 LC/MSD VL system; column: Zorbax Eclipse XDB-C18 Rapid Resolution (4.6×75 mm, 3.5 μm, USUT002736); temperature: 25° C.; starting pressure: 105 bar; flow rate: 1.0 mL/min; mobile phase (% A=0.025% trifluoroacetic acid in H₂O, % B=0.025% trifluoroacetic acid in acetonitrile) Gradient program: 70% A/30% B (start), step gradient to 50% B over 5 min, step gradient to 98% B over 8.30 min, hold at 98% B over 25.20 min, step gradient to 30% B over 25.40 min; PDA Detector: 470 nm. The presence of trifluoroacetic acid in the LC eluents acts to protonate synthesized lutein disuccinate and diphosphate salts to give the free di-acid forms, yielding M⁺=768 for the disuccinate salt sample and M⁺=728 for the diphosphate salt sample in MS analyses. LRMS: +mode; ESI: electrospray chemical ionization, ion collection using quadrapole; APCI: atmospheric pressure chemical ionization, ion collection using quadrapole. MS (ESI-IT) was recorded on a HCT plus Bruker Daltonics Mass Spectrometer system, LRMS: +mode; ESI-IT: electrospray chemical ionization, ion collection using ion trap. ¹H NMR analyses were attempted on Varian spectrometers (300 and 500 MHz). NMR analyses of natural source lutein as well as synthesized lutein derivatives yielded only partially discernable spectra, perhaps due to the presence of interfering impurities (natural source lutein), or due to aggregation (natural source lutein and derivatives). In attempts to circumvent the problems associated with NMR analyses, samples were prepared using mixtures of deuterated solvents including methanol/chloroform, methanol/water, methyl sulfoxide/water, and chloroform/methanol/water. However, such attempts failed to give useful data.

Natural source lutein (β,ε-carotene-3,3′-diol), 1. LC/MS (ESI): 9.95 min (2.78%), λₘₐₓ 226 nm (17%), 425 nm (100%); 10.58 min (3.03%), λₘₐₓ 225 nm (21%), 400 nm (100%); 11.10 min (4.17%), λₘₐₓ 225 nm (16%), 447 nm (100%); 12.41 min (90.02%), λₘₐₓ 269 nm (14%), 447 nm (100%), m/z 568 M⁺ (69%), 551 [M−H₂O+H]⁺ (100%), 533 [M−2H₂O+H]⁺ (8%).

β,ε-carotenyl 3,3′-disuccinate, 2. To a solution of natural source lutein (1) (0.50 g, 0.879 minol) in CH₂Cl₂ (8 mL) was added N,N-diisopropylethylamine (3.1 mL, 17.58 mmol) and succinic anhydride (0.88 g, 8.79 mmol). The solution was stirred at RT overnight and then diluted with CH₂Cl₂ and quenched with water/1 M HCl (5/1). The aqueous layer was extracted two times with CH₂Cl₂ and the combined organic layer was washed three times with cold water/1 M HCI (5/1), dried over Na₂SO₄, and concentrated. The resulting red-orange oil was washed (slurried) three times with hexanes to yield disuccinate 2 (0.433 g, 64%) as a red-orange solid; LC/MS (APCI): 10.37 min (4.42%), λₘₐₓ 227 nm (56%), 448 nm (100%), m/z 769 [M+H]⁺ (8%), 668 [M−C₄O₃H₄]⁺ (9%), 637 (36%), 138 (100%); 11.50 min (92.40%), λₘₐₓ 269 nm (18%),447 nm (100%), m/z 769 [M+H]⁺ (7%), 668 [M−C₄O₃H₄]⁺ (9%), 651 (100%); 12.03 min (3.18%) λₘₐₓ 227 nm (55%), 446 nm (100%), m/z 668 [M−C₄O₃H₄]⁺ (15%), 550 (10%), 138 (100%)

β,ε-carotenyl 3,3′-disuccinate sodium salt, 3. To a solution of disuccinate 2 (0.32 g, 0.416 mmol) in CH₂CL₂/methanol (5 mL/1 mL ) at 0° C. was added drop-wise sodium methoxide (25% wt in methanol; 0.170 mL, 0.748 mmol). The solution was stirred at RT overnight and then quenched with water and stirred for 5 min. The solution was then concentrated and the aqueous layer was washed four times with Et₂O. Lyophilization of the clear, red-orange aqueous solution yielded 3 (0.278 g, 91%) as an orange, hygroscopic solid; LC/MS (APCI): 11.71 min (94.29%), λₘₐₓ 269 nm (18%), 446 nm (100%), m/z 769 [M−2Na+3H]⁺ (8%), 668 [M−2Na+2H−C₄O₃H₄]⁺ (6%), 651 (100%); 12.74 min (5.71%), λₘₐₓ 227 nm (30%), 269 nm (18%), 332 nm (39%), 444 nm (100%), m/z 768 [M−2Na+2H]⁺ (2%), 668 [M−2Na+2H−C₄O₃H₄]⁺ (3%), 651 (12%), 138 (100%)

Tribenzyl phosphite, 4. To a well-stirred solution of phosphorus trichioride (1.7 mL, 19.4 mmol) in Et₂O (430 nL) at 0° C. was added dropwise a solution of triethylamine (8.4 mL, 60.3 mmol) in Et₂O (20 mL) a solution of benzyl alcohol (8.1 mL, 77.8 mmol) in Et₂O (20 mL). The mixture was stirred at 0° C. for 30 min and then at RT overnight. The mixture was filtered and the filtrate concentrated to give a colorless oil. Silica chromatography (hexanes/Et₂O/triethylamine, 4/1/1%) of the crude product yielded 4 (5.68 g, 83%) as a clear, colorless oil that was stored under N₂ at −20° C.; ¹H NMR: δ 7.38 (15H, m), 4.90 (6H, d)

Dibenzyl phosphoroiodidate, 5. To a solution of tribenzyl phosphite (5.43 g, 15.4 mmol) in CH₂Cl₂ (8 mL) at 0° C. was added I₂ (3.76 g, 14.8 mmol). The mixture was stirred at 0° C. for 10 min or until the solution became clear and colorless. The solution was then stirred at RT for 10 min and used directly in the next step.

3-(Bis benzyl-phosphoryloxy)-3′-(phosphoryloxy)-β,ε-carotene, 6. To a solution of natural source lutein (1) (0.842 g, 1.48 mmol) in CH₂Cl₂ (8 mL) was added pyridine (4.8 mL, 59.2 mmol). The solution was stirred at 0° C. for 5 min and then freshly prepared 5 (14.8 mmol) in CH₂Cl₂ (8 mL) was added drop-wise to the mixture at 0° C. The solution was stirred at 0° C. for 1 h and then diluted with CH₂Cl₂ and quenched with brine. The aqueous layer was extracted twice with CH₂Cl₂ and the combined organic layer was washed once with brine, then dried over Na₂SO₄ and concentrated. Pyridine was removed from the crude red oil by azeotropic distillation using toluene. The crude product was alternately washed (slurried) twice with hexanes and Et₂O to yield 6 as a red oil, used in the next step without further purification; LC/MS (ESI): 9.93 min (44.78%), λₘₐₓ 267 nm (33%), 444 nm (100%), m/z 890 [M−H₂O]⁺ (8%), 811 [M−PO₃H−H₂O+H]⁺ (73%), 533 (100%); 9.99 min (29.0%), λₘₐₓ 268 nm (24%), 446 nm (100%), m/z 890 [M−H₂O]⁺ (6%), 811 [M−PO₃H−H₂O+H]⁺ (72%), 533 (100%); 10.06 min (26.23%), λₘₐₓ 266 nm (15%), 332 nm (22%), 444 nm (100%), m/z 890 [M−H₂O]⁺ (5%), 811 [M−PO₃H−H₂O+H]⁺ (90%), 533 (100%)

3-(Bis benzyl-phosphoryloxy)-3′-hydroxy-β,ε-carotene, 7. To a solution of 6 (0.033 nmmol) in tetrahydrofuran/water (1 mL/0.5 mL) at 0° C. was added LiOH—H₂O (0.003 g, 0.073 mmol). The solution was stirred at RT for 1 h and then quenched with methanol. The crude reaction mixture was analyzed by LC/MS; LC/MS (ESI): 10.02 min (40.60%), λₘₐₓ 266 nm (12%), 333 nm (25%), 445 nm (100%), m/z 890 [M−H₂O]⁺ (33%), 811 [M−PO₃H−H₂O+H]⁺ (50%), 533 (100%); 16.37 min (49.56%) λₘₐₓ 267 nm (16%), 332 nm (27%), 446 nm (100%), m/z 828 M⁺ (55%), 550 (44%)

3,340 -Diphosphoryloxy-β,ε-carotene, 8. To a solution of 6 (1.48 mmol) in CH₂Cl₂ (10 mL) at 0° C. was added drop-wise N,O-bis(trimethylsilyl)acetamide (3.7 mL, 14.8 mmol) and then bromotrimethylsilane (1.56 mL, 11.8 mmol). The solution was stirred at 0° C. for 1 h, quenched with methanol, diluted with CH₂Cl₂, and then concentrated. The resulting red oil was alternately washed (slurried) three times with ethyl acetate and CH₂Cl₂ to yield crude phosphate 8 (2.23 g) as a dark orange oil, used in the next step without further purification; LC/MS (ESI): 8.55 min (45.67%), λₘₐₓ 214 nm (25%), 268 nm (28%), 447 nm (100%), m/z 631 [M−PO₃H−H₂O+H]⁺ (30%), 533 (18%), 279 (13%), 138 (87%); 8.95 min (35.0%), λₘₐₓ 217 nm (14%), 268 nm (23%), 448 nm (100%), m/z 631 [M−PO₃H−H₂O+H]⁺ (26%), 533 (32%), 279 (18%), 138 (100%); 9.41 min (9.70%), λₘₐₓ 225 nm (37%), 269 nm (23%), 335 nm (19%), 447 nm (100%), m/z 631 [M−PO₃H−H₂O+H]⁺ (6%), 533 (18%), 279 (13%), 138 (100%)

3,3′-Diphosphoryloxy-β,ε-carotene sodium salt, 9. To a solution of crude 8 (ca 50%; 2.23 g, 3.06 mmol) in methanol (20 mL) at 0° C. was added drop-wise sodium methoxide (25%; 3.5 mL, 15.3 mmol). The solution was stirred at RT for 2 h and the resulting orange solid was washed (slurried) three times with methanol. Water was added to the moist solid and the resulting aqueous layer was extracted with CH₂Cl₂, ethyl acetate, and again with CH₂Cl₂. Lyophilization of the clear, red-orange aqueous solution yielded 9 (0.956 g, 80% over 3 steps) as an orange, hygroscopic solid; LC/MS (ESI): 7.81 min (22.34%), λₘₐₓ 215 nm (34%), 268 nm (30%), 448 nm (100%), m/z 711 [M−-4Na−H₂O+5H]⁺ (9%), 533 (13%), 306 (100%); 8.33 min (39.56%), λₘₐₓ 217 nm (14%), 268 nm (20%), 448 nm (100%), m/z 711 [M−4Na−H₂O+5H]⁺ (10%), 533 (11%), 306 (100%); 8.90 min (38.09%), λₘₐₓ 223 nm (45%), 269 nm (30%), 336 nm (26%), 448 nm (100%), m/z 711 [M−4Na−H₂O+5H]⁺ (8%), 631 [M−4Na−PO₃H−H−H₂O+5H]⁺ (18%), 533 (20%), 306 (100%); MS (ESI-IT): m/z 816 M⁺ (55%), 772 [M−2Na+2H]⁺ (37%), 728 [M−4Na+4H]⁺ (74%)

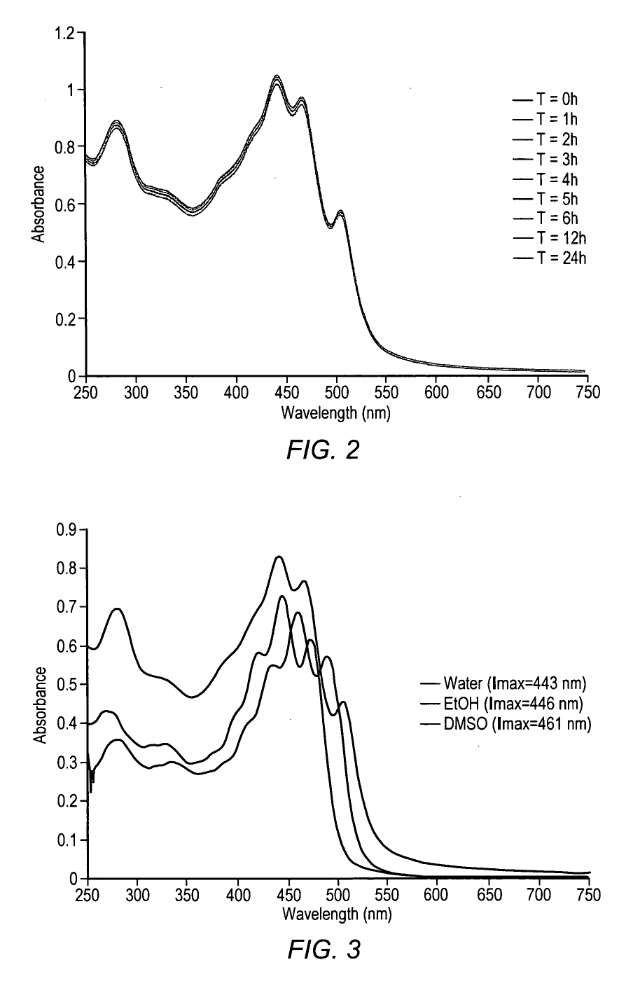

UV/Visible spectroscopy. For spectroscopic sample preparations, 3 and 9 were dissolved in the appropriate solvent to yield final concentrations of approximately 0.01 mM and 0.2 mM, respectively. The solutions were then added to a rectangular cuvette with 1 cm path length fitted with a glass stopper. The absorption spectrum was subsequently registered between 250 and 750 nm. All spectra were accumulated one time with a bandwidth of 1.0 nm at a scan speed of 370 nm/min. For the aggregation time-series measurements, spectra were obtained at baseline (immediately after solvation; time zero) and then at the same intervals up to and including 24 hours post-solvation (see FIG. 2-FIG. 7). Concentration was held constant in the ethanolic titration of the diphosphate lutein sodium salt, for which evidence of card-pack aggregation was obtained (FIG. 5-FIG. 7).

Determination of aqueous solubility/dispersibility. 30.13 mg of 3 was added to 1 mL of USP-purified water. The sample was rotated for 2 hours, then centrifuged for 5 minutes. After centrifuging, solid was visible in the bottom of the tube. A 125-μL aliquot of the solution was then diluted to 25 mL. The sample was analyzed by UV/Vis spectroscopy at 436 nm, and the absorbance was compared to a standard curve compiled from 4 standards of known concentration. The concentration of the original supernatant was calculated to be 2.85 mg/mL and the absorptivity was 36.94 AU*mL/cm*mg. Slight error may have been introduced by the small size of the original aliquot.

Next, 30.80 mg of 9 was added to 1 mL of USP-purified water. The sample was rotated for 2 hours, then centrifuged for 5 minutes. After centrifuging, solid was visible in the bottom of the tube. A 125-μL aliquot of the solution was then diluted to 25 mL. The sample was analyzed by UViis spectroscopy at 411 nm, and the absorbance was compared to a standard curve compiled from 4 standards of known concentration. The concentration of the original supernatant was calculated to be 29.27 mg/mL and the absorptivity was 2.90 AU*mL/cm*mg. Slight error may have been introduced by the small size of the original aliquot.

Leukocyte Isolation and Preparation. Human polymorphonuclear leukocytes (PMNs) were isolated from freshly sampled venous blood of a single volunteer (S.F.L.) by Percoll density gradient centrifugation as described previously. Briefly, each 10 mL of whole blood was mixed with 0.8 mL of 0.1 M EDTA and 25 mL of saline. The diluted blood was then layered over 9 mL of Percoll at a specific density of 1.080 g/mL. After centrifugation at 400×g for 20 min at 20° C., the plasma, mononuclear cell, and Percoll layers were removed. Erythrocytes were subsequently lysed by addition of 18 mL of ice-cold water for 30 s, followed by 2 mL of 10×PIPES buffer (25 mM PIPES, 110 mM NaCl, and 5 mM KCl, titrated to pH 7.4 with NaOH). Cells were then pelleted at 4° C., the supernatant was decanted, and the procedure was repeated. After the second hypotonic cell lysis, cells were washed twice with PAG buffer [PIPES buffer containing 0.003% human serum albumin (HSA) and 0.1% glucose]. Afterward, PMNs were counted by light microscopy on a hemocytometer. The isolation yielded PMNs with a purity of >95%. The final pellet was then suspended in PAG-CM buffer (PAG buffer with 1 mM CaCl₂ and 1 mM MgCl₂).

EPR Measurements. All EPR measurements were performed using a Bruker ER 300 EPR spectrometer operating at X-band with a TM₁₁₀ cavity as previously described. The microwave frequency was measured with a Model 575 microwave counter (EIP Microwave, Inc., San Jose, Calif.). To measure superoxide anion (O₂) generation from phorbol-ester (PMA)-stimulated PMNs, EPR spin-trapping studies were performed using the spin trap DEPMPO (Oxis, Portland, Oreg.) at 10 mM. 1×10⁶ PMNs were stimulated with PMA (1 ng/mL) and loaded into capillary tubes for EPR measurements. To determine the radical scavenging ability of 3 and 9 in aqueous and ethanolic formulations, PMNs were pre-incubated for 5 minutes with test compound, followed by PMA stimulation.

Instrument settings used in the spin-trapping experiments were as follows: modulation amplitude, 0.32 G; time constant, 0.16 s; scan time, 60 s; modulation frequency, 100 kHz; microwave power, 20 milliwatts; and microwave frequency, 9.76 GHz. The samples were placed in a quartz EPR flat cell, and spectra were recorded. The component signals in the spectra were identified and quantified as reported previously.

UV/V is Spectral Properties in Organic and Aqueous Solvents.

UV-V is spectral evaluation of the disuccinate lutein sodium salt is depicted in FIG. 2-FIG. 4. FIG. 2 depicts a time series of the UV/V is absorption spectra of the disodium disuccinate derivative of natural source lutein in water. The λₘₐₓ (443 nm) obtained at time zero did not appreciably blue-shift over the course of 24 hours, vibrational fine structure was maintained (% III/II=35%), and the spectra became only slightly hypochromic (i.e. decreased in absorbance intensity) over time, indicating minimal time-dependent supramolecular assembly (aggregation) of the card-pack type during this time period. Existence of head-to-tail (J-type) aggregation in solution cannot be ruled out.

FIG. 3 depicts a UV/V is absorption spectra of the disodium disuccinate derivative of natural source lutein in water (λₘₐₓ =443 nm), ethanol (λₘₐₓ =446 nm), and DMSO (λₘₐₓ =461 nm). Spectra were obtained at time zero. A prominent cis peak is seen with a maximum at 282 nm in water. The expected bathochromic shift of the spectrum in the more polarizable solvent (DMSO) is seen (461 nm). Only a slight hypsochromic shift is seen between the spectrum in water and that in ethanol, reflecting minimal card-pack aggregation in aqueous solution. Replacement of the main visible absorption band observed in EtOH by an intense peak in the near UV region—narrow and displaying no vibrational fine structure—is not observed in the aqueous solution of this highly water-dispersible derivative, in comparison to the spectrum of pure lutein in an organic/water mixture.

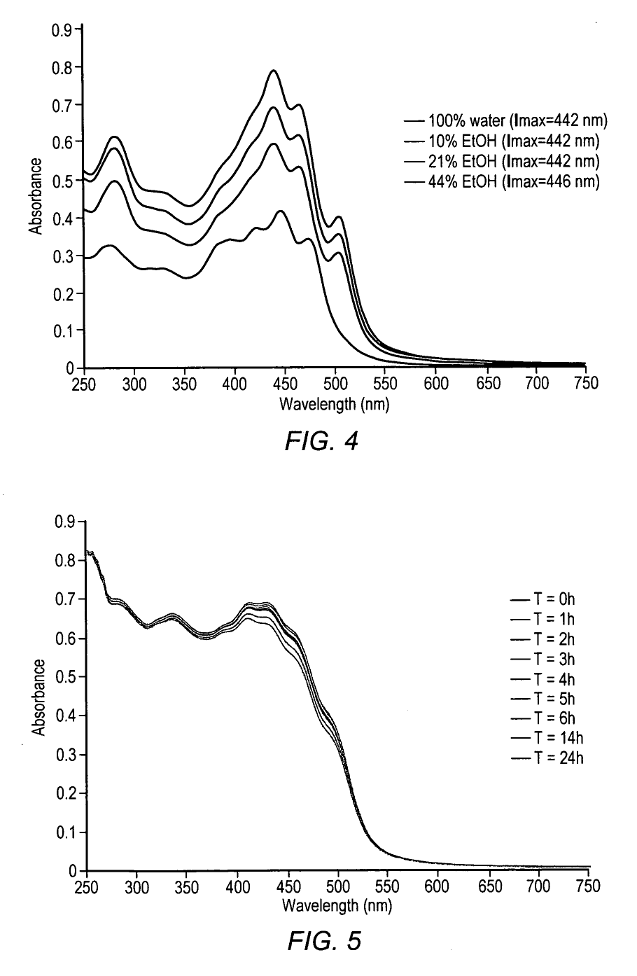

FIG. 4 depicts a UV/V is absorption spectra of the disodium disuccinate derivative of natural source lutein in water (λₘₐₓ =442 nm) with increasing concentrations of ethanol. The λₘₐₓ increases to 446 nm at an EtOH concentration of 44%, at which point no fturther shift of the absorption maximum occurs (i.e. a molecular solution has been achieved), identical to that obtained in 100% EtOH (See FIG. 3).

UV-V is spectral evaluation of the diphosphate lutein sodium salt is depicted in FIG. 5-FIG. 7. FIG. 5 depicts a time series of the UV/V is absorption spectra of the disodium diphosphate derivative of natural source lutein in water. Loss of vibrational fine structure (spectral distribution beginning to approach unimodality) and the blue-shifted lambda max relative to the lutein chromophore in EtOH suggested that card-pack aggregation was present immediately upon solvation. The λₘₐₓ (428 nm) obtained at time zero did not appreciably blue-shift over the course of 24 hours, and the spectra became slightly more hypochromic over time (i.e. decreased in absorbance intensity), indicating additional time-dependent supramolecular assembly (aggregation) of the card-pack type during this time period. This spectrum was essentially maintained over the course of 24 hours (compare with FIG. 2, disuccinate lutein sodium salt).

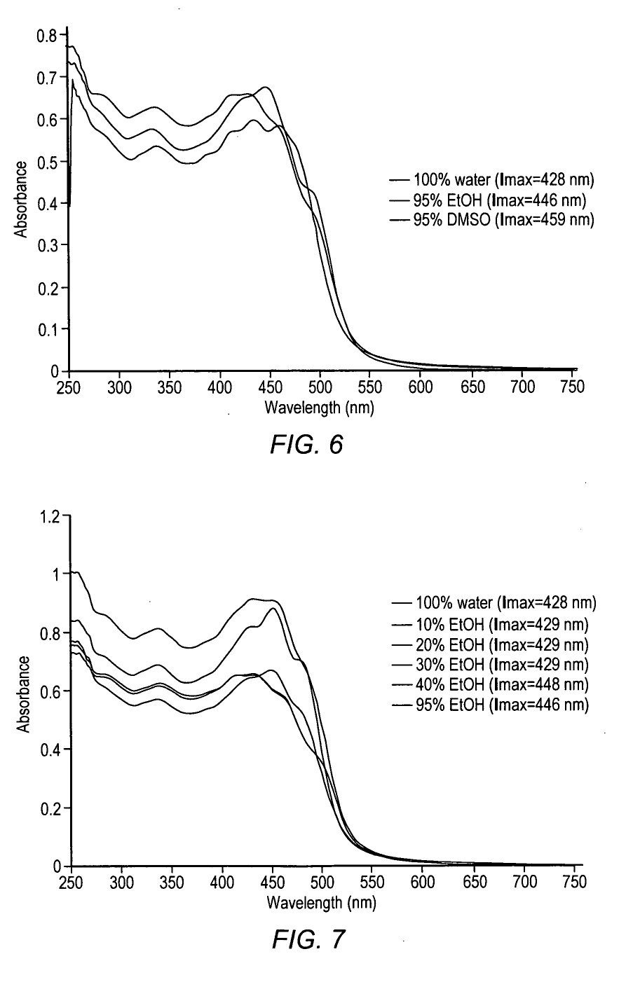

FIG. 6 depicts a UV/V is absorption spectra of the disodium diphosphate derivative of natural source lutein in 95% ethanol (λₘₐₓ =446 nm), 95% DMSO (λₘₐₓ =459 nm), and water (λₘₐₓ =428 Mn). A red-shift was observed (λₘₐₓ to 446 nm), as was observed with the disuccinate derivate. Wetting of the diphosphate lutein derivative with a small amount of water was required to obtain appreciable solubility in organic solvent (e.g. EtOH and DMSO). Spectra were obtained at time zero. The expected bathochromic shift (in this case to 459 nm) of the spectrum in the more polarizable solvent (95% DMSO) is seen. Increased vibrational fine structure and red-shifting of the spectra were observed in the organic solvents.

FIG. 7 depicts a UV/V is absorption spectra of the disodium diphosphate derivative of natural source lutein in water (λₘₐₓ =428 nm) with increasing concentrations of ethanol. Concentration of the derivative was held constant for each increased concentration of EtOH in solution. The λₘₐₓ increases to 448 nm at an EtOH concentration of 40%, at which no further shift of the absorption maximum occurs (i.e. a molecular solution is reached).

Direct superoxide Anion Scavenging by EPR Spectroscopy

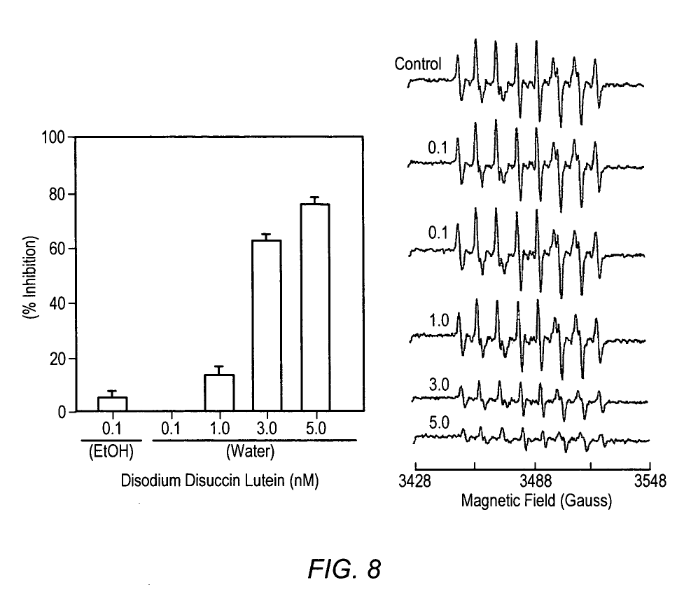

The mean percent inhibition of superoxide anion signal (±SEM) as detected by DEPMPO spin-trap by the disodium disuccinate derivative of natural source lutein (tested in water) is shown in FIG. 8. A 100 μM formulation (0.1 mM) was also tested in 40% EtOH, a concentration shown to produce a molecular (i.e. non-aggregated) solution. As the concentration of the derivative increased, inhibition of superoxide anion signal increased in a dose-dependent manner. At 5 mM, approximately ¾ (75%) of the superoxide anion signal was inhibited. No significant scavenging (0% inhibition) was observed at 0.1 mM in water. Addition of 40% EtOH to the derivative solution at 0.1 mM did not significantly increase scavenging over that provided by the EtOH vehicle alone (5% inhibition). The millimolar concentration scavenging by the derivative was accomplished in water alone, without the addition of organic co-solvent (e.g., acetone, EtOH); heat, detergents, or other additives. This data suggested that card-pack aggregation for this derivative was not occurring in aqueous solution (and thus limiting the interaction of the aggregated carotenoid derivative with aqueous superoxide anion).

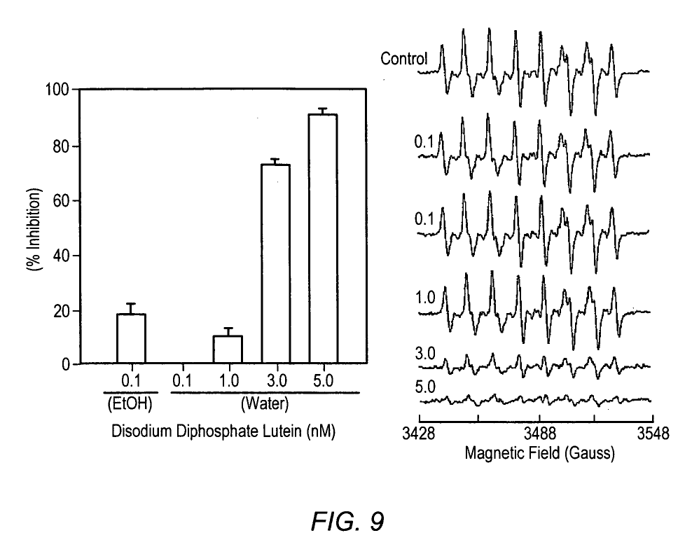

The mean percent inhibition of superoxide anion signal (±SEM) as detected by DEPMPO spin-trap by the disodium diphosphate derivative of natural source lutein (tested in water) is shown in FIG. 9. A 100 μM formulation (0.1 mM) was also tested in 40% EtOH, a concentration also shown to produce a molecular (i.e. non-aggregated) solution of this derivative. As the concentration of the derivative increased, inhibition of the superoxide anion signal increased in a dose-dependent manner. At 5 mM, slightly more than 90% of the superoxide anion signal was inhibited (versus 75% for the disuccinate lutein sodium salt). As for the disuccinate lutein sodium salt, no apparent scavenging (0% inhibition) was observed at 0.1 mM in water. However, a significant increase over background scavenging by the EtOH vehicle (5%) was observed after the addition of 40% EtOH , resulting in a mean 18% inhibition of superoxide anion signal. This suggested that disaggregation of the compound lead to an increase in scavenging ability by this derivative, pointing to slightly increased scavenging ability of molecular solutions of the more water-dispersible diphosphate derivative relative to the disuccinate derivative. Again, the millimolar concentration scavenging by the derivative was accomplished in water alone, without the addition of organic co-solvent (e.g., acetone, EtOH), heat, detergents, or other additives.

TABLE I
Descriptive statistics of mean % inhibition of superoxide anion signal for aqueous and ethanolic (40%)
formulations of disodium disuccinate derivatives of natural source lutein tested in the current study.
Sample sizes of 3 were evaluated for each formulation, with the exception of natural source lutein in
40% EtOH stock solution (N = 1). Mean % inhibition did not increase over background levels until
sample concentration reached 1 mM in water; likewise, addition of 40% EtOH at the 0.1 mM
concentration did not increase scavenging over background levels attributable to the EtOH vehicle
(mean = 5% inhibition).

| | | | | Mean (% | | | | | |
|---|---|---|---|---|---|---|---|---|---|
| Sample | Solvent | Concentration | N | inhibition) | S.D. | SEM | Min | Max | Range |
| Lutein Disuccinate | 40% | 0.1 mM | 3 | 5.0 | 4.4 | 2.5 | 0 | 8 | 8 |
| Sodium Salt | EtOH | | | | | | | | |
| Lutein Disuccinate | Water | 0.1 mM | 1 | 0.0 | ND | ND | 0 | 0 | 0 |
| Sodium Salt | | | | | | | | | |
| Lutein Disuccinate | Water | 1.0 mM | 3 | 13.0 | 5.6 | 3.2 | 8 | 19 | 11 |
| Sodium Salt | | | | | | | | | |
| Lutein Disuccinate | Water | 3.0 mM | 3 | 61.7 | 4.0 | 2.3 | 58 | 66 | 8 |
| Sodium Salt | | | | | | | | | |
| Lutein Disuccinate | Water | 5.0 mM | 3 | 74.7 | 4.5 | 2.6 | 70 | 79 | 9 |
| Sodium Salt | | | | | | | | | |

TABLE II
Descriptive statistics of mean % inhibition of superoxide anion signal for aqueous and ethanolic
(40%) formulations of disodium diphosphate derivatives of natural source lutein tested in the current
study. Sample sizes of 3 were evaluated for each formulation, with the exception of lutein diphosphate
in water at 100 μM (0.1 mM) where N = 1. Mean % inhibition of superoxide anion signal increased
in a dose-dependent manner as the concentration of lutein diphosphate was increased in the test assay.
At 100 μM in water, no inhibition of scavenging was seen. The molecular solution in 40% EtOH
(mean % inhibition = 18%) was increased above background scavenging (5%) by the ethanolic
vehicle, suggesting that disaggregation increased scavenging at that concentration. Slightly increased
scavenging (on a molar basis) may have been obtained with the diphosphate derivative in
comparison to disuccinate derivative (see Table 1 and FIG. 8).

| | | | | Mean (% | | | | | |
|---|---|---|---|---|---|---|---|---|---|
| Sample | Solvent | Concentration | N | inhibition) | S.D. | SEM | Min | Max | Range |
| Lutein Diphosphate | 40% | 0.1 mM | 3 | 18.0 | 7.0 | 4.0 | 11 | 25 | 14 |
| Sodium Salt | EtOH | | | | | | | | |
| Lutein Diphosphate | Water | 0.1 mM | 1 | 0.0 | ND | ND | 0 | 0 | 0 |
| Sodium Salt | | | | | | | | | |
| Lutein Diphosphate | Water | 1.0 mM | 3 | 9.3 | 3.5 | 2.0 | 6 | 13 | 7 |
| Sodium Salt | | | | | | | | | |
| Lutein Diphosphate | Water | 3.0 mM | 3 | 72.3 | 3.1 | 1.8 | 69 | 75 | 6 |
| Sodium Salt | | | | | | | | | |
| Lutein Diphosphate | Water | 5.0 mM | 3 | 91.0 | 2.6 | 1.5 | 88 | 93 | 5 |
| Sodium Salt | | | | | | | | | |

In the current study, facile preparatsion of the disodium disuccinate and tetrasodium phosphate esters of natural source (RRR) latein are described. These asymmetric C40 carotenoid derivates exhibited aqueous dispersibility of 2.85 and 29.27 mg/mL, respectively. Evidence for bothe card-pack (H-type) and head-to-tail (J-type) supramolecular assembly was obtained with UV-V is spectroscopy for the aqueous solution of these compounds. Electronic paramagnetic spectroscopy for the aqueous superoxide scavenging by these derivatives demonstrated nearly identical dose-dependent scavenging profiles, with slightly increased scavenging noted for the diphosphate derivative. In each case, scavenging in the millimolar range was observed. These results show that as parenteral soft drugs with aqeous radical scavenging activity, both compounds are useful in those clinical applications in which rapid and/or intravenous delivery is desired for the desired therapeutic effect(s).

Tetrasodium Diphosphate Astaxanthin Derivatives General. The racemic tetrasodium diphosphate derivative of astaxanthin (pAST; >97% purity by HPLC) was synthesized from commercial astaxanthin and its structure verified (see below for synthetic methodology). The chemical structures of the three stereoisomers, (3R,3′R)—, (3S,3—S)—, and (3R,3′S; meso)-tetrasodium diphosphate astaxanthin are shown in FIG. 10. The racemic pAST used in this study is comprised of the statistical mixture of the above stereoisomers in a 1:1:2 ratio. Non-esterified, all-E synthetic astaxanthin utilized for biological tests (AST; >96% purity by HPLC) was obtained from Sigma (St. Louis, Mo.). Canthaxanthin (CTX) and the synthetic retinoid tetrahydrotetramethylnaphalenylpropenyl benzoic acid (TTNPB) were gifts from Hoffman-LaRoche (Nutley, N.J.). TTNPB, canthaxanthin and astaxanthin concentrations were confirmed by comparing their UV absorption and their published extinction coefficients. Due to the sensitivity of carotenoids to light, heat and oxygen, special precautions were taken throughout the study. All compounds were stored under nitrogen at −70° C. and care was taken to ensure minimal exposure to direct sunlight or UV light.

Synthesis of tetrasodium diphosphate astaxanthin (pAST). Unless otherwise noted, all reagents and solvents were purchased from commercial suppliers and used as received without further purification. Proton and carbon nuclear magnetic resonance (NMR) spectra were obtained on a Bruker AMX 500 spectrometer at 500 MHz for proton NMR (¹H NMR) and 202 MHz for phosphorous NMR (³¹P NMR). Chemical shifts are given in ppm (δ) and coupling constants, J, are reported in Hertz (Hz). Tetramethylsilane (TMS) was used as an internal standard for proton spectra. High performance liquid chromatography (HPLC) analysis for in-process control (IPC) was performed on a Varian Prostar Series 210 liquid chromatograph with a PDA detector using methods A and B. Method A: Waters Symmetry C18, 3.5 μm, 4.6×150 mm column; 25° C.; Mobile phase: [A=water (pH 4.5, 20 mM ammonium acetate), B=acetonitrile], 95% A/5% B (start); hold for 5 min; linear gradient to 100% B over 25 min; hold for 8 min; linear gradient to 5% B over 2 min; flow rate: 1.0 ml/min; detector wavelength: 474 nm (astaxanthin 30.5 min). Method B: Agilent Zorbax SB CN 5 μm, 4.6×150 mm column; 25° C.; Mobile phase: [A=water (pH 6.8, 20 mM ammonium acetate), B=acetonitrile], 80% A/20% B (start); hold for 5 min; linear gradient to 100% B over 20 min; hold for 10 min; linear gradient to 20% B over 5 min; flow rate: 1.0 ml/min; detector wavelength: 474 nm (astaxanthin, 30.5 min).

The intermediate rac-3,3′-dihydroxy-β,β-carotene4,4′-dione diphosphoric acid bis-(2-cyano-ethyl) ester was synthesized as follows: a 100 ml round bottom flask wrapped with aluminum foil was equipped with a stir bar under nitrogen at room temperature. Racemic astaxanthin (Buckton Scott, India) (0.440 g, 0.74 mmol) was dissolved in methylene chloride (13.2 ml) then reacted with bis (2-cyanoethyl)-N,N-diisopropyl phosphoramidite (0.80 g, 2.95 mmol), and tetrazole (0.21 g, 2.95 mmol). After 14 h, the reaction was complete by HPLC and 7.4 ml 0.4 M iodine in a solution of pyridine-dichloromethane (DCM)-water (3:1:1) was added dropwise over 15 min. The reaction was diluted with DCM (10 ml) and washed with aqueous sodium thiosulfate (1 M, 2×10 ml) and brine (10 ml). The solution was concentrated to dryness to afford 590 mg of dried red solid (83% yield). ¹H NMR (CDCl₃) δ 6.42-6.17 (14 H, m), 5.12-5.06 (2 H, m), 4.59-4.31 (m, 8 H), 2.93-2.83 (8 H, m), 2.07-1.98 (16 H,m), 1.89 (6 H, s), 1.35 (6 H, s), 1.24 (6 H, s); ³¹P NMR (CDCl₃) δ −2.62; Anal. Calculated for [C₅₂H₆₆O₁₀P₂]: 969.05, ESI MS m/z 969.29 [C₅₂H₆₆O₁₀P₂]⁺; HPLC (Method B) 94.0% area under the curve (AUC), t_{R}=26.0 min.

rac-3,3′-dihydroxy-β,β-carotene-4,4′-dione diphosphoric acid bis-(2-cyano-ethyl) (500 mg, 0.52 mmol) and 10 ml of 50% dimethylamine in water was added to a 250 ml flask under nitrogen (N₂) with a stir bar and heated to 40° C. The reaction was complete by HPLC after 4d and the reaction mixture was concentrated to dryness. The red solid was re-dissolved in 50 ml of water and then eluted through a sodium ion exchange resin (50 g, Amberlite IR-120 Na+). The solution was concentrated using acetonitrile to form an azeotrope with water. The red solid was then re-dissolved in a minimum amount of water (−20 ml) and precipitated with the addition of ethanol (20 ml). The precipitate was filtered through a 2 μm filter and dried under vacuum to afford 66 mg of red solid. The solid was again re-dissolved in 2 ml of water and lyophilized to afford 42 mg of red solid (28% yield). ¹H NMR (CD₃OD) δ 6.81-6.35 (14 H, m), 4.87-4.83 (2 H, m), 2.07-1.98 (16 H, m), 1.90 (6 H, s), 1.30 (6 H, s), 1.14 (6 H, s); ³¹P NMR (CDCl₃) δ 3.28; Anal. Calculated for [C₄₀H₅₄O₁₀P₂]: 756.80, ESI MS m/z 755.2 [C₄₀H₅₃O₁₀P₂]-; HPLC (Method B) 97.7% AUC, t_{R}=14.05 min.

Determination of aqueous solubility/dipsersibility of pAST. 23.70 mg of tetrasodium diphosphate astaxanthin was added to 1 ml of USP purified water. The mixture was stirred for 2 hours, and centrifuged for 5 minutes. The solution was diluted in water and analyzed by UV/vis spectroscopy at 480 nm and absorbance was compared to a standard curve compiled from 4 standards of known concentration. The solubility was calculated to be 25.21 mg/ml with an extinction coefficient of 0.0187 AU*ml/cm*mg.

Cell lines and culture conditions. Mouse embryonic fibroblast 10T1/2 cells were cultured in Eagle's basal medium with Earle's salts supplemented with 4% fetal calf serum (Atlanta Biologicals, Norcross, Ga.), 25 μg/ml gentamicin sulfate (Sigma, St. Louis, Mo.), and passaged by trypsin/EDTA (Gibco Invitrogen, Carlsbad, Calif.) and maintained at 37° C. in a 5% CO₂ atmosphere. The cells were allowed to grow until a monolayer was formed. The confluent cells were treated 7 days after seeding, unless otherwise indicated. dAST was prepared in a formulation of 20% EtOH and sterile water (0.2% final EtOH ) to minimize supramolecular aggregation, and the final concentration of EtOH in culture medium was 0.2%. CTX was dissolved in THF and added to media. TTNPB was dissolved in acetone (Sigma, St. Louis, Mo.) and cultures received a final acetone concentration of 0.1%.

Treatments/Compounds._TTNPB (Biomol, Plymouth Meeting, Pa.); stock 5×10⁻⁶M in acetone, diluted 1:500 in culture media (final 10⁻⁸ M in 0.2% acetone). Lycophyll (All-trans, 95% pure, Hawaii Biotech, Inc., Aiea, Hi.); stocks 10⁻² M and 10⁻³ M in tetrahydroftiran (THF; Sigma, St. Louis, Mo.) diluted 1:1000 and stirred into culture media immediately before treatment (final 10⁻⁵ M and 10⁻⁶ M in 0.1% THF). Lycopene (92.7% pure, Chromadex, Inc., Santa Ana, Calif.); stocks 10⁻² M and 10⁻³ M in tetrahydrofuran (Sigma, St. Louis, Mo.) diluted 1:1000 and stir into culture media immediately before treatment (final 10⁻⁵ M and 10⁻⁶ M in 0.1% THF). Homochiral 3S,3 ′S-astaxanthin (95% pure, Hawaii Biotech, Inc., Aiea, Hi.); stocks 10⁻² M and 10⁻³ M in THF (Sigma, St. Louis, Mo.) diluted 1:1000 and stirred into culture media immediately before treatment (final 10⁻⁵ M and 10⁻⁶ M in 0.1% THF).

SDS-PAGE Electrophoresis, Transfer and Immunodetection. Cell were trypsinized and pelleted briefly. Pellets were lysed in phosphate buffered saline (PBS) containing protease inhibitor cocktail (Roche, Nutley, N.J.; 1 tablet/10 mL), 10 mM sodium fluoride, 0.5 mM sodium vanadate, 4 mM para-methyl-sulfonyl fluoride and 0.5% sodium dodecylsulfate. Lysates were sonicated and protein concentrations quantified using the BCA protein determination assay (Pierce, Rockford, Ill.). Equal amounts of total protein were boiled in sample buffer (Fisher, Fairlawn, N.J.) containing 10% β-mercaptoethanol, loaded onto 10% Tris-Glycine gels (Cambrex, East Rutherford, N.J.) and run at 115 V for 1.5 hours using Tris (25 mM)/Glycine (192 mM)/SDS (0.1%) running buffer. Protein standards were utilized to confirm molecular weight of detected protein (SeeBlue, Invitrogen, Carlsbad, Calif.). Protein was transferred from SDS-PAGE gels to PVDF membranes (Invitrogen, Carlsbad, Calif.) using Tris (19 mM)/Glycine (144 mM)/10% Methanol/0.1% SDS buffer at 33 volts for 2 hours. Cx43 protein was detected according to the manufacturer's instructions using the WesternBreeze Immunodetection Kit (Invitrogen, Carlsbad, Calif.) and a rabbit primary antibody reactive against the cytoplasmic tail of Cx43 (1:2000, Sigma, St. Louis, Mo.).

Quantification of Relative Western Blot Cx43 Protein Levels. Digital scans of immunodetected membranes were analyzed for relative intensity of Cx43 protein bands using the public domain densitometry program NIH Image J and presented as relative fold inductions standardized to THF only control levels.

### Example 1

Analysis of CX43 protein expression. Expression of CX43 protein in 10T1/2 cells was assessed by Western blotting. 10T1/2 cell monolayers were treated with the indicated carotenoid derivatives or with retinoids (as a positive control for the modulation of CX43 expression) 7 days after seeding in 100 mm dishes (Fisher Scientific, Pittsburgh, Pa.). Fours days after the drug was added to the cells, the cells were harvested, total cellular protein was isolated and the total protein concentration thereof was determined using a commercially available Protein Assay Reagent kit (Pierce Chemical Co., Rockford, Ill.). 40 μg of total cellular protein was resolved on an SDS-PAGE gel, transferred to a nitrocellulose membrane, and analyzed by Western blotting using the NuPage Western blotting kit (Invitrogen, Carlsbad, Calif.). CX43 was detected using a rabbit polyclonal antibody (Zymed, San Francisco, Calif.) raised against a synthetic polypeptide corresponding to the C-terminal domain common to mouse, human and rat CX43. Equal protein loafing was verified by immunoblotting for GAPDH, the expression of which is unaffected by treatment with retinoids or carotenoids, using a rabbit polyclonal GAPDH antibody was also used (Zymed, San Francisco, Calif.). CX43 and GAPDH immunoreactive bands were visualized by chemiluminescence using an anti-rabbit HRP-conjugated secondary antibody (Pierce Chemical Co., Rockford, Ill.). Images were obtained by exposure to X-ray film as previously described [26] and scanned for digital analysis on the Fluoro-S Imager (Bio-Rad, Richmond, Calif.).

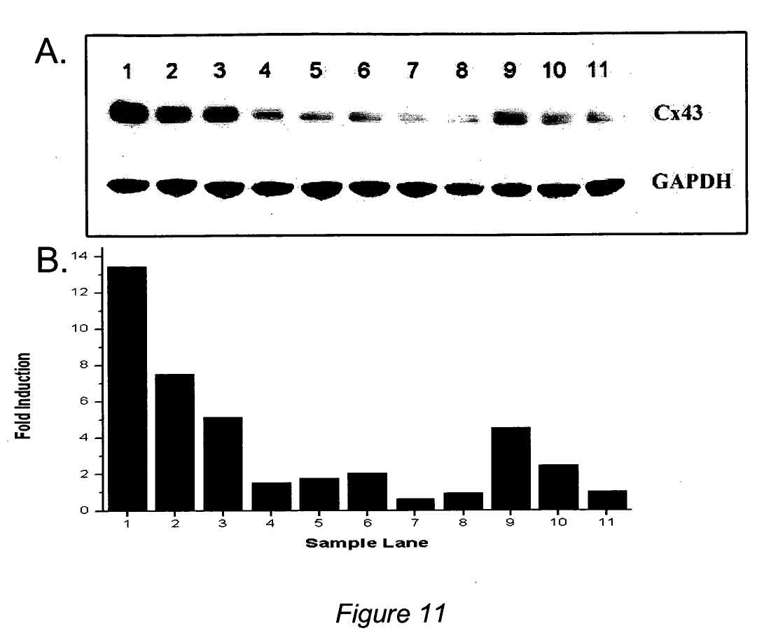

Results. Turning to FIG. 11, racemic pAST increased the level of detectable CX43 protein in cells in comparison with solvent-treated controls at concentrations of 10⁻⁶ and 10⁻⁷ M, and was equipotent to CTX at these concentrations (about 5- and 2-fold induction, respectively). CTX, included as a positive carotenoid control, was active at 10⁻⁵ M as had been previously observed (≈7-fold induction). No change in CX43 protein levels were detectable in cells treated with identical concentrations of AST. Surprisingly, no change in protein levels was observed in cells treated with 10⁻⁵ M of either compound, suggesting potential toxicity of the compounds at high concentrations. As expected, CX43 expression was increased about 13-fold by the synthetic retinoid TTNPB at 10⁻⁸ M included as positive control. These results demonstrate that the novel water-soluble carotenoids delivered in an aqueous ethanol formulation are superior to AST itself, delivered in THF, in modulating CX43 protein levels.

### Example 2

Analysis of CX43 protein by indirect immunofluorescence. Expression and assembly of CX43 into plaques was assessed by immunofluorescence staining essentially as described in Rogers et al, 1990, which is incorporated herein by reference. Briefly, confluent cultures of 10T1/2 cells were grown on Permanox plastic 4-chamber slides (Nalge Nunc International, Naperville, Ill.) and treated for 4 days as described above. Cells were fixed with −20° C. methanol overnight, washed in buffer, blocked in 1% bovine serum albumin (Sigma, St. Louis, Mo.) in PBS, incubated with the rabbit anti-CX43 antibody, and visualized with Alexa568 conjugated anti-rabbit secondary antibody (Molecular Probes, Eugene, Oreg.). Images were acquired with a Zeiss Axioplan microscope and a Roper Scientific cooled CCD camera.

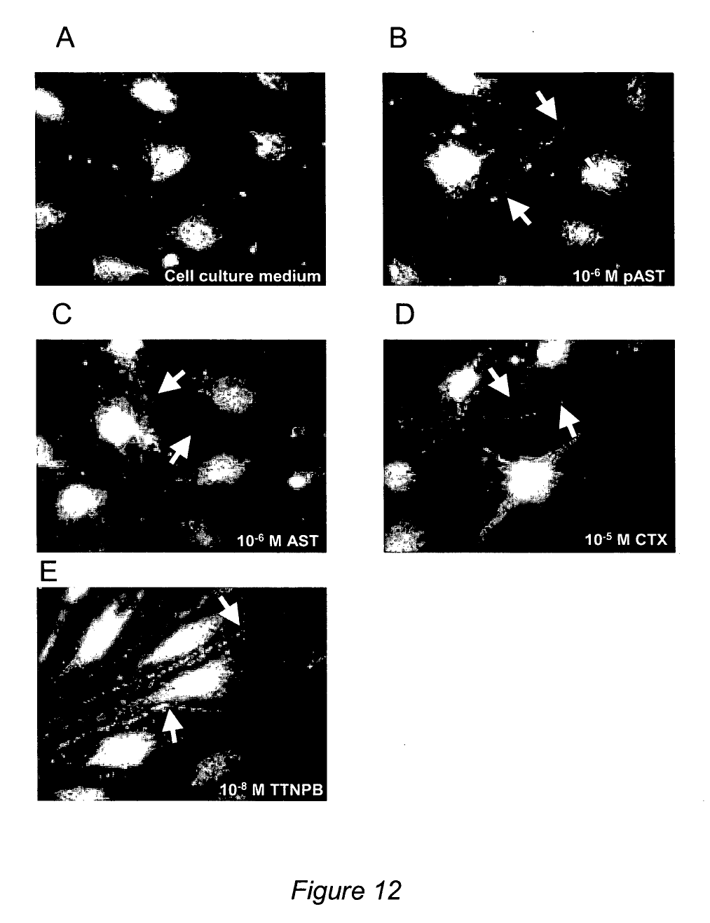

Results. It has previously been demonstrated that monolayer cultures of 10T1/2 cells have relatively low levels of CX43 protein. Consequently, CX43 immunoreactive plaques, corresponding to assembled gap junctions, are infrequent. Turning to FIG. 12, treatment 10T1/2 cell monolayers with racemic pAST at 10⁻⁶ M (panel B) increased the prevalence of CX43 immunoreactive plaques in regions of cell/cell apposition when compared to cells in untreated control cultures (panel A). In contrast, few immunoreactive plaques were observed in cultures treated with AST at 10⁻⁶ M (panel C); the frequency of which is lower than in untreated monolayers. At the lowest concentration of AST (10⁻⁸ M; not shown) tested, gap junction assembly was comparable to untreated cultures. Cells treated with TTNPB at 10⁻⁸ M (panel E), as expected, exhibited extensive CX43 immunoreactive plaques, while treatment with CTX at 10⁻⁵ M (panel D) resulted in a prevalence of CX43 immunoreactive plaques that was roughly equivalent to that achieved in cells treated with pAST at 10⁻⁶ M, indicating that pAST is more efficient than CTX or AST at modulating the number of gap junctions in a cell.

Gap junctional communication assay. Junctional permeability was assayed by the scrape-loading dye transfer assay essentially as described in El-Fouly et al., 1987, which is incorporated herein by reference. Briefly, confluent cultures of 10T1/2 cells grown in 60 mm dishes were treated with the indicated compounds for 7 days. The treated cells were washed with Ca⁺²-free phosphate-buffered saline (PBS). 1.5 ml of Lucifer Yellow CH (Sigma, St. Louis, Mo.) 0.2% in PBS was then added, and linear cuts were made on the monolayer using a surgical scalpel. The cultures were incubated for 2 minutes at 37° C. then rinsed thoroughly with PBS. The cultures were then fixed with 2 ml of 5% formaldehyde in PBS. Images were digitally quantitated by intensity thresholding using the SigmaScan software program (Jandel Scientific, San Rafael, Calif.).

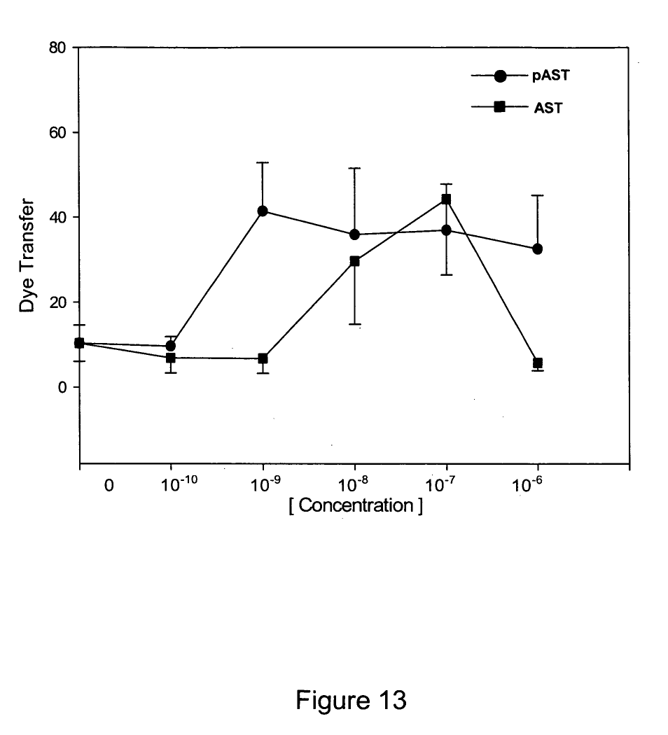

Results. Turning to FIG. 13, racemic pAST (●-●) or AST (▪-▪) was added to monolayer cultures at concentrations ranging from 10⁻¹⁰ to 10⁻⁶ M as indicated. At a concentration of 10⁻⁹ M, pAST increased the level of GJIC approximately 4-fold over that seen in untreated controls cultures, or cultures treated with AST. The relative amount of GJIC in pAST-treated cells remained constant over a 4-log concentration range, indicating that maximal GJIC induction by pAST can be achieved at low concentration. At a concentration of 10⁻⁶ M AST, dye-transfer in monolayer cultures is below that observed in untreated control cultures. At a concentration of 10⁻⁷ M AST, dye transfer roughly equivalent to that achieved in cultures treated with pAST. Therafter, communication decreased in a dose-responsive manner. The positive controls CTX 10⁻⁵ M and TTNPB 10⁻⁸ M increased dye transfer 4-fold and 6-fold respectively (not shown).

Inhibition of MCA-induced neoplastic transformation in 10T1/2 cells. Cells were initiated with methylcholanthrene (MCA) 5 μpg/ml (Sigma, St. Louis, Mo.) in acetone for 24 hours. Potential inhibitors of neoplastic transformation were added 7 days after removal of carcinogen as indicated and were renewed weekly by adding fresh medium supplemented with the appropriate drug for four weeks after removal of the carcinogen. The cultures were fixed and stained as described above. A total of 24 dishes of cells were used per treatment group. Type II and III foci were identified and then quantitated as described in Bertram et al., 1990, which is incorporated herein by reference.

Results. In each case, AST or pAST was added to monolayer cultures 7 days after the removal of the carcinogen MCA so as not to potentially interfere with the production of carcinogen-initiated cells. Results are presented as the mean number of foci per dish (Table 3). In control cultures treated with acetone only, about 1 focus/6 dishes was observed (mean 0.17 foci/dish); this was increased to 1.67 foci/dish in cultures exposed to MCA (P>0.0002). No foci were observed in MCA-induced cells treated with 10⁻⁶ M pAST. Cell treated with 10⁻⁷ M and 10⁻⁸ M pAST showed substantially lowered levels of neoplastic transformation compared to untreated cells (P<0.04). AST at all concentrations tested inhibited transformation to about 40% of control (0.98 foci/dish) in a dose-independent manner. Transformation was strongly inhibited in cells treated with either pAST or AST at 10⁻⁵ M for 4 weeks, however these cultures failed to form complete monolayers, potentially indicating cumulative toxicity at this concentration.

Selective cytotoxicity. Determination of plating efficiencies and growth rates were performed as described in Pung et al., 1988, which is incorporated herein by reference. Briefly, normal 10T1/2 cells and MCA-transformed 10T1/2 cells were plated onto 100 mm plates at a density of 10⁴/dish and treated 24 h later with either pAST, AST or EtOH-only as controls. Cells from duplicate cultures were trypsinized after 1, 2, 6 and 8 days, and the density of cells in the cultures was determined using a Coulter counter (Coulter Electronics, Inc., Hialeah, Fla.).

Cellular uptake of carotenoid derivatives. Cellular levels of astaxanthin in pAST- and AST-treated cells were determined in confluent 10T1/2 cells treated with pAST or AST at 10⁻⁶ M or cells treated with media-alone as a control. Duplicate cultures of cells were treated as described above. After 1, 4 and 7 days after treatment with the indicated compounds, the cells were harvested by trypsinization, pelleted by centrifugation and snap frozen in liquid nitrogen. The frozen cell pellets subjected to HPLC analysis to determine the cytosolic concentration of the compound. Astaxanthin was extracted from the samples as essentially as described in Showalter et al., 2004, with slight modifications. Methanol (1.0 ml) and water (1.0 ml) were added to each sample weighed in advance, and then mixed with an Ultraturax® mixer for 20 seconds. Chloroform (3 ml) was added to the samples, and the samples were mixed for 20 seconds. Finally, a saturated sodium chloride (NaCl) solution (1 ml) was added to each sample, after which the samples were mixed for an additional 20 seconds. The samples were allowed to sit for 5 min to allow particulate matter to settle to the bottom of the tube. The samples were then centrifuged (1700×g, 10 min). The chloroform phase containing astaxanthin (2 ml was transferred to a clean test tube and the chloroform was evaporated on a heating block (40° C.) with a gentle flow of N₂ gas. The residue was then dissolved in n-hexane:acetone (86:14; 75 μl) and transferred directly into sample vials. Total astaxanthin, including all-E-, 9Z- and 13Z-astaxanthin, was quantified by HPLC using a phosphoric acid-modified silica gel column, with all-E-astaxanthin as an external standard. The flow was 1 ml/min and the detection wavelength was set at 470 nm. The employed extinction coefficients (E_{1 cm, 1%}) at 472 nm in hexane containing 4% chloroform were 2100 for all-E-astaxanthin, and 1350 and 1750 for 13Z- and 9Z-astaxanthin, respectively.

TABLE 3

| Compound | Concentration | Foci/dish^{a} | % Inhibition^{b} |
|---|---|---|---|
| MCA | 5 μg/ml | 1.63 | N/A |
| +THF | 0.1% | 1.92 | N/A |
| 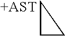 | 10⁻⁶ M 10⁻⁷ M 10⁻⁸ M | 0.96 1.00 0.96 | 41* 39 41* |
| 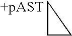 | 10⁻⁶ M 10⁻⁷ M 10⁻⁸ M | 0 0.91 1.04 | 100** 44* 36* |

Inhibitory effects of carotenoids on MCA-induced transformation 10T1/2 cells were exposed to MCA as carcinogen, then 7 days after removal, received the indicated carotenoids for a 4 week period.
^{a}Mean number of Type II + Type III foci in a total of 24 dishes. 0.17 foci/dish were observed in dishes not receiving MCA.
^{b}Calculated with respect to THF controls. Statistical Significance:
**denotes highly significant (P-value < 0.000006);
*(P < 0.04). There was no significant difference between MCA-alone and MCA then THF-treated controls.

Statistical analysis. Transformation data was analyzed by one-tailed, two-sample t-tests that incorporated unequal variances. The scrape-loading data was analyzed by paired t-tests.

### Example 3

Previous studies have demonstrated that treatment of C3H10T1/2 immortalized embryonic mouse fibroblast cells and normal human fibroblasts with several carotenoids including lycopene and ‘racemic’ (i.e. the statistical mixture of stereoisomers) astaxanthin results in elevated protein levels of the gap junction protein, Connexin43 (Cx43) (Bertram 1999). Here, we show that treatment of the same mouse fibroblast cell line with 10⁻⁵ M, 10⁻⁶ M and 10⁻⁷ M lycophyll for seven days also resulted in increased Cx43 protein levels. Lycophyll at 10⁻⁵ M appeared to induce Cx43 protein increases equivalently to 10⁻⁵ M homochiral (3S,3′S) astaxanthin and 10⁻⁵ M, 10⁻⁶ M lycopene. This is the second such study utilizing these compounds in the mouse fibroblast system for which upregulation of Cx43 has been reported, with slight methods modifications as summarized below. Lycophyll from total synthesis in this case was tested as a mixture of geometric isomers (cis and trans), and the utility here for upregulation of Cx43 supports the previous demonstration of activity for all-trans lycophyll prepared by semi-preparative chromatrography (Jackson et al. 2005).

Immortalized mouse fibroblast cells (C3H10T1/2) were cultured in Dulbecco's Modification of Eagle's Medium (DMEM) containing 5% calf serum (Mediatech Inc.) and Penicillin (200 i.u.)/Streptomycin (200 μg/ml, Mediatech, Inc.) and incubated at 37 degrees C. (° C.) in 5% CO₂/air atmosphere. Cell dissociation was performed utilizing trypsin:EDTA (0.25%: 2.21 mM, Mediatech Inc.).

TTNPB (Biomol, Plymouth Meeting, Pa.); stock 5×10⁻⁶M in acetone, diluted 1:500 in culture media (final 10⁻⁸ M in 0.2% acetone). Lycophyll (All-trans, 95% pure, Hawaii Biotech, Inc., Aiea, Hi.); stocks 10⁻² M and 10⁻³ M in tetrahydrofuran (THF; Sigma, St. Louis, Mo.) diluted 1:1000 and stirred into culture media immediately before treatment (final 10⁻⁵M and 10⁻⁶M in 0.1% THF). Lycopene (92.7% pure, Chromadex, Inc., Santa Ana, Calif.); stock 10⁻² M and 10⁻³ M in tetrahydrofuran (Sigma, St. Louis, Mo.) diluted 1:1000 and stirred into culture media immediately before treatment (final 10⁻⁵ M and 10⁻⁶ M in 0.1% THF). Homochiral 3S,340 S-astaxanthin (95% pure, Hawaii Biotech, Inc., Aiea, Hi.); stocks 10⁻² M and 10⁻³ M in THF (Sigma, St. Louis, Mo.) diluted 1:1000 and stirred into culture media immediately before treatment (final 10⁻⁵ M and 10⁻⁶ M in 0.1% THF).

Cell were trypsinized and pelleted briefly. Pellets were lysed in phosphate buffered saline (PBS) containing protease inhibitor cocktail (Roche, Nutley, N.J.; 1 tablet/10 mL), 10 mM sodium fluoride, 0.5 mM sodium vanadate, 4 mM para-methyl-sulfonyl fluoride and 0.5% sodium dodecylsulfate. Lysates were sonicated and protein concentrations quantified using the BCA protein determination assay (Pierce, Rockford, Ill.). Equal amounts of total protein were boiled in sample buffer (Fisher, Fairlawn, N.J.) containing 10% β-mercaptoethanol, loaded onto 10% Tris-Glycine gels (Cambrex, East Rutherford, N.J.) and run at 115 V for 1.5 hours using Tris (25 mM)/Glycine (192 mM)/SDS (0.1%) running buffer. Protein standards were utilized to confirm molecular weight of detected protein (SeeBlue, Invitrogen, Carlsbad, Calif.). Protein was transferred from SDS-PAGE gels to PVDF membranes (Invitrogen, Carlsbad, Calif.) using Tris (19 mM)/Glycine (144 mM)/10% Methanol/0.1% SDS buffer at 33 volts for 2 hours. Cx43 protein was detected according to the manufacturer's instructions using the WesternBreeze Immunodetection Kit (Invitrogen, Carlsbad, Calif.) and a rabbit primary antibody reactive against the cytoplasmic tail of Cx43 (1:2000, Sigma, St. Louis, Mo.).

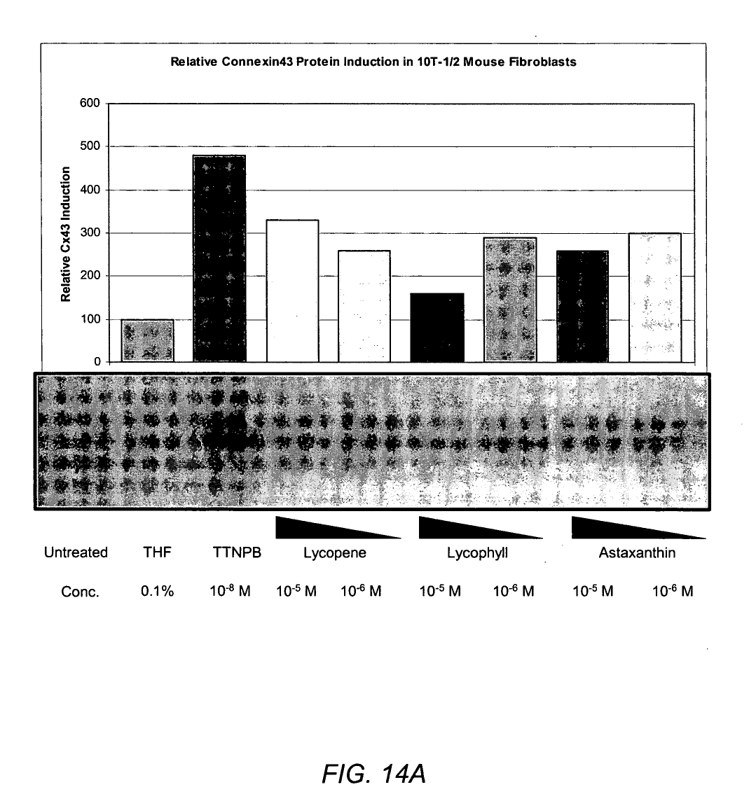

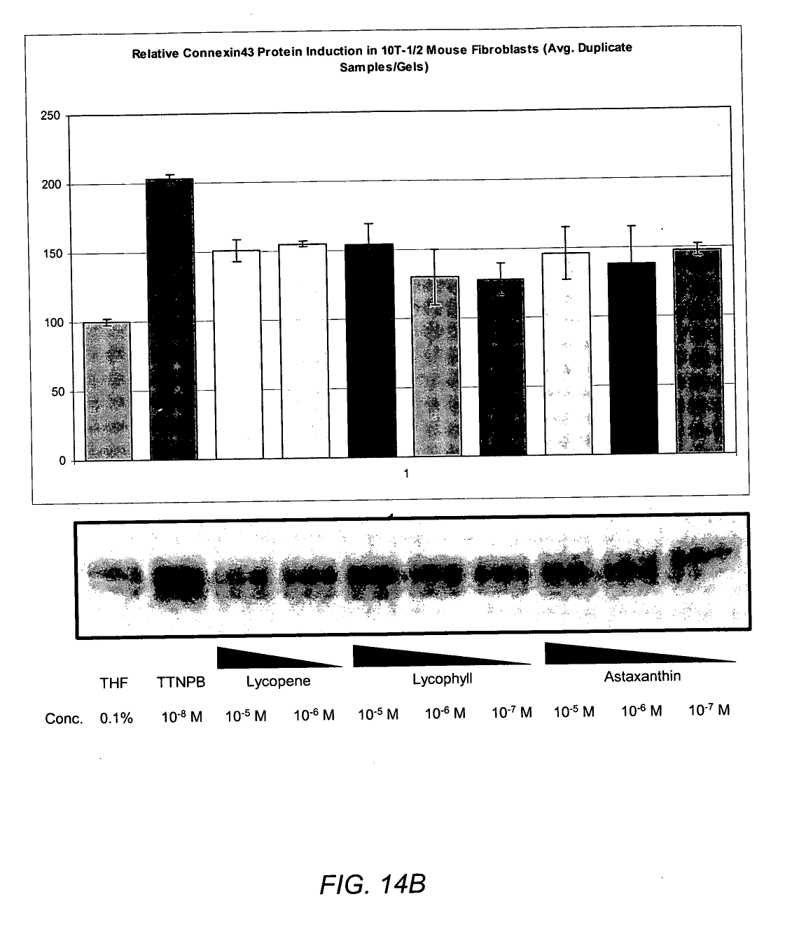

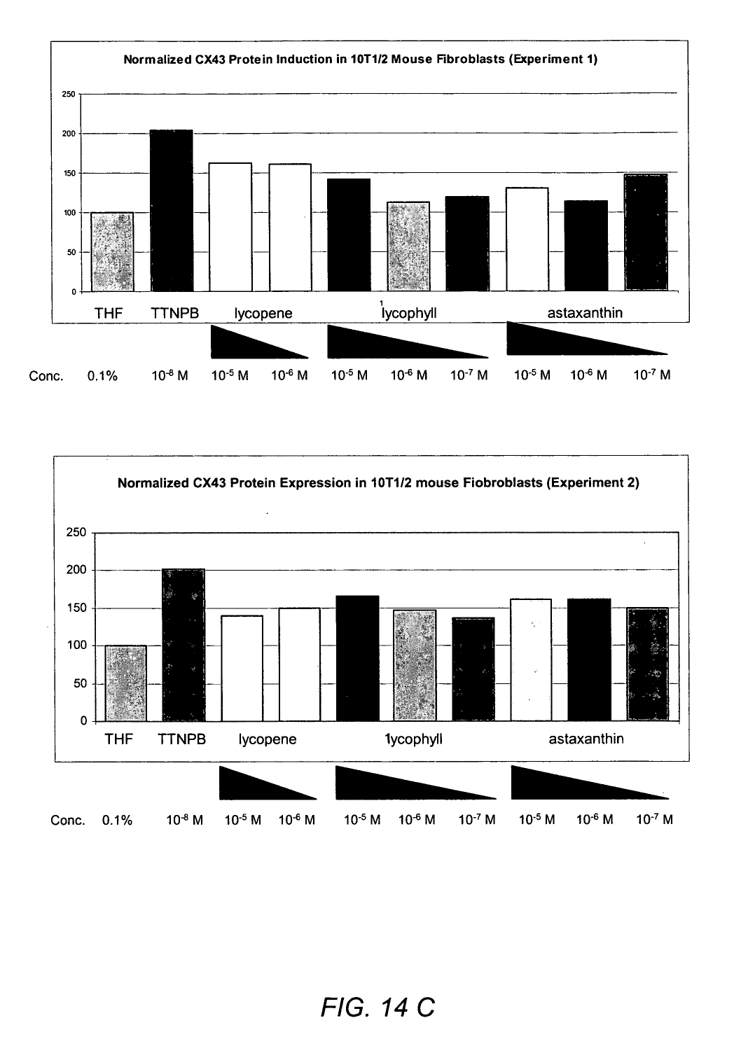

Digital scans of immunodetected membranes were analyzed for relative intensity of Cx43 protein bands using the public domain densitometry program NIH Image J and presented as relative fold inductions standardized to THF only control levels. The results of individual experiments are shown in FIG. 14A and FIG. 14B. The results were normalized to the expression of Cx43 in 10T1/2 cells treated with vehicle alone and are summarized graphically in FIG. 14C.

Results: The results presented in FIGS. 14A-14C demonstrate once again that lycophyll (in this case a mixture of geometric isomers) is capable of upregulating Cx43 expression in mouse embryonic fibroblast cells. The relative 5 inductions (in duplicate) are consistent with induction by the comparable carotenoids lycopene (positive control) and homochiral (3S,3′S) astaxanthin. The synthetic retinoid TTNPB demonstrates characteristic strong induction of Cx43 in this system. Cx43 is a tumor suppressor gene that has utility in cancer chemoprevention, and its modulation by the naturally-occurring lycophyll compound is novel and suggests potential clinical utility in the setting of cancer chemoprevention and treatment. Results are also summarized in Table 4.

TABLE 4

| Compound | Concentration | Fold induction^{a} |
|---|---|---|
| THF | 0.1% | 1.0 |
| +TTNPB | 10⁻⁸ M | 2.03 |
| 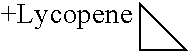 | 10⁻⁶ M 10⁻⁵ M | 1.55 1.51 |
| 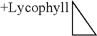 | 10⁻⁷ M 10⁻⁶ M 10⁻⁵ M | 1.28 1.30 1.54 |
| 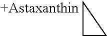 | 10⁻⁷ M 10⁻⁶ M 10⁻⁵ M | 1.48 1.38 1.46 |

Increased expression of Cx43 in transformed cells after treatment with the indicated synthetic carotenoid analog or derivative.
^{a}Taken from an average of two independent experiments and normalized using Cx43 expresssion in the presence of THF as baseline.

### Example 4

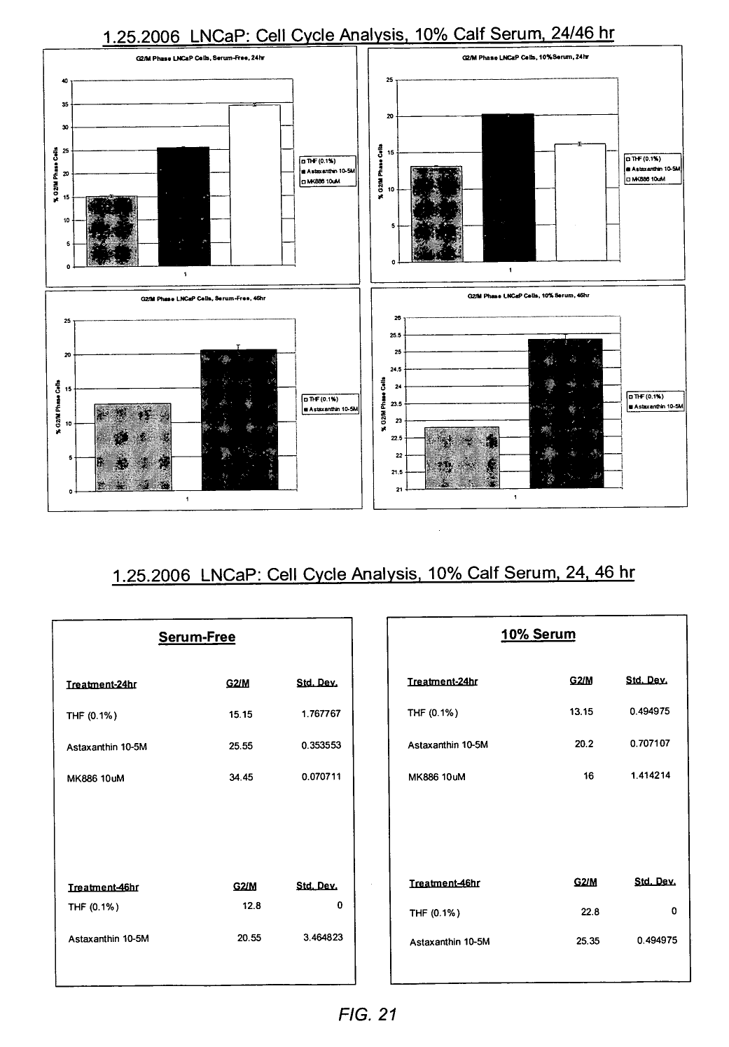

In order to evaluate the efficacy of certain carotenoid analogs and derivatives for induction of apoptosis in cancer cells, the compounds were applied in various concentrations to a transformed human cell line derived from a malignant prostate tumor that had metastasized to the lymph nodes of a prostate cancer patient (LNCaP cells). The cells were cultured in vitro using standart art-recognized methods. The cells were treated with doses of lycophyll, lycopene or astaxanthin at concentrations ranging from 10⁻⁵-10⁻⁷ M for 1-72 hours. As a negative control to detect were left untreated, or were treated with the the pharmacologic vehicle THF. As a positive control to detect apoptosis, cells were administered 10⁻⁸ M of the 5-LO/FLAP inhibitor MK-866. Cells were harvested and the DNA was stained with propidium iodide according to methods widely known in the art. The stained cells were subjected to flow cytometry and the percentage of cells having sub G1 amounts levels of DNA (corresponding to apoptotic bodies) as well as those cells that were growth arrested in G2/M were determined. The results obtained from these experiments are shown in FIGS. 15-21. All carotenoids tested were able to induce apoptosis in at least a portion of the cells that were administered the compound.

These data therefore demonstrate that the subject carotenoid analogs or derivatives are are effective agents for use in the treatment of proliferative disorders. These data further demonstrate that the subject carotenoid analogs or derivatives affect the growth and survival of neoplastic cells through at least two two distinct mechanisms, namely, through upregulation of Cx43 and GJIC, and by inducing apoptosis, likely through inhibition of the 5-LO pathway. As such, they may be considered as both “chemopreventive” (i.e. used for the prevention of neoplastic disease) and “chemotherapeutic” (i.e. used for the amelioration of disease once established) agents.

In this patent, certain U.S. patents, U.S. patent applications, and other materials (e.g., non-U.S. patents and journal articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description to the invention. Changes may be made in the elements described herein without departing from the spirit and scope of the invention as described in the following claims. In addition, it is to be understood that features described herein independently may, in certain embodiments, be combined.

1-39. (canceled)

40. A method of treating a proliferative disorder in a subject comprising administering to a subject who would benefit from such treatment a therapeutically effective amount of a pharmaceutical composition that facilitates induction of apoptosis in cancer cells, wherein the pharmaceutical composition comprises at least one carotenoid analog or derivative having the structure; 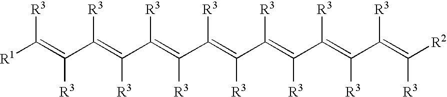
where each R³ is independently hydrogen or methyl, and where each R¹ and R² are independently: 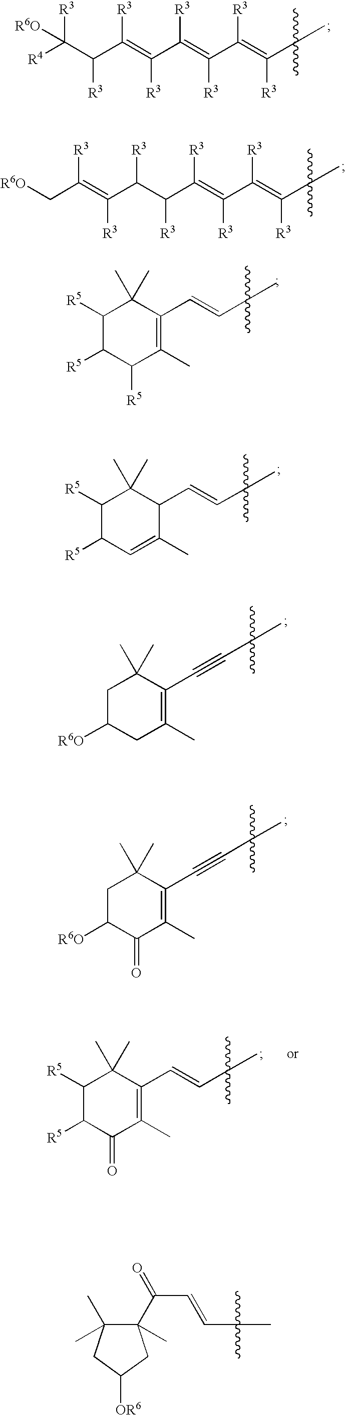
where R⁴ is hydrogen or methyl; where each R⁵ is independently hydrogen, —OH, or —OR⁶ wherein at least one R⁵ group is —OR⁶; wherein each R⁶ is independently: alkyl; aryl; -alkyl-N(R⁷)₂; -aryl-N(R⁷)₂; —N⁺(R⁷)₃; -aryl-N⁺(R⁷)₃; -alkyl-CO₂R⁷; -aryl-CO₂R⁷; -alkyl-CO₂⁻; -aryl-CO₂⁻; —O—C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl or a co-antioxidant; where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

41. The method of claim 40, wherein the proliferative disorder is a cancer.

42. The method of claim 40, wherein the proliferative disorder is selected from the list consisting of pancreatic cancer; bladder cancer; colorectal cancer; breast cancer; metastatic breast cancer; prostate cancer; androgen-dependent prostate cancer; androgen-independent prostate cancer; renal cancer; metastatic renal cell carcinoma; hepatocellular cancer; lung cancer, non-small cell lung cancer (NSCLC); bronchioloalveolar carcinoma (BAC); adenocarcinoma of the lung; ovarian cancer; progressive epithelial cancer; primary peritoneal cancer; cervical cancer; gastric cancer; esophageal cancer; head and neck cancer; squamous cell carcinoma of the head and neck; melanoma; neuroendocrine cancer; metastatic neuroendocrine tumor; brain cancer; glioma, anaplastic oligodendroglioma; adult glioblastoma multiforme; adult anaplastic astrocytoma; bone cancer; and soft tissue sarcoma.

43. (canceled)

44. The method of claim 40, further comprising administering to the subject an anti-cancer agent.

45. The method of claim 44, wherein the anticancer agent is a DNA-damaging agent, an agent that disrupts cell replication, a proteasome inhibitor, an NF-κB inhibitor, an IKK inhibitor, a topoisomerase I inhibitor, irinotecan, topotecan, camptothecin, doxorubicin, topoisomerase II inhibitor, etoposide, teniposide, daunorubicin, an alkylating agent, melphalan, chlorambucil, busulfan, thiotepa, ifosfamide, carmustine, lomustine, semustine, streptozocin, decarbazine, methotrexate, mitomycin C, and cyclophosphamide, a DNA intercalator, cisplatin, oxaliplatin, carboplatin, a free-radical generator, bleomycin, a nucleoside mimetics, 5-fluorouracil, capecitibine, gemcitabine, fludarabine, cytarabine, mercaptopurine, thioguanine, pentostatin, and hydroxyurea, paclitaxel, docetaxel, vincristine, vinblastin, thalidomide, CC-5013, CC-4047, a protein tyrosine kinase inhibitor, imatinib mesylate and gefitinib, an antibody that binds specifically to antigens expressed on the surface of cancer cells, trastuzumab, rituximab, cetuximab, bevacizumab, or analogs, derivatives or metabolite thereof.

46. The method of claim 44, wherein the carotenoid analog or derivative and the anticancer agent are administered concurrently.

47. The method of claim 44, wherein the carotenoid analog or derivative and the anticancer agent are administered separately.

48-50. (canceled)

51. The method of claim 40, wherein one or more carotenoid derivatives or analogs have the structure: 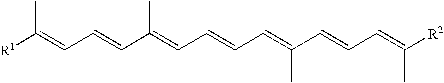
where each R¹ and R² are independently: 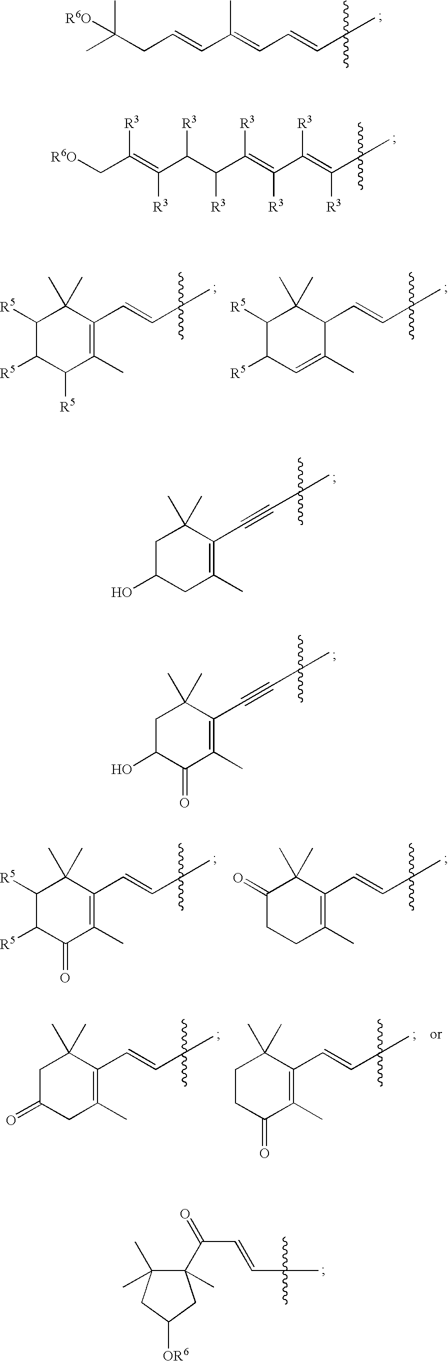
where each R⁵ is independently hydrogen, —OH, or —OR⁶ wherein at least one R⁵ group is —OR⁶; wherein each R⁶ is independently: allyl; aryl; -alkyl-N(R⁷)₂; -aryl-N(R⁷)₂; -alkyl-N⁺(R⁷)₃; -aryl-N⁺(R⁷)₃; -alkyl-CO₂R⁷; -aryl-CO₂R⁷; -alkyl-CO₂⁻; -aryl-CO₂⁻; —O—C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl, or a co-antioxidant; and where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

52. The method of claim 40, wherein one or more carotenoid derivatives or analogs have the structure: 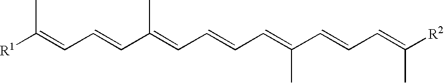
where each R¹ and R² are independently: 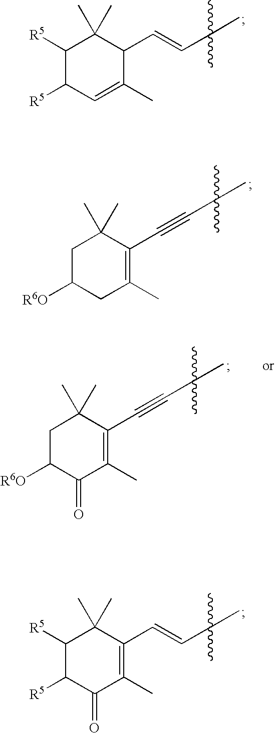
where each R⁵is independently hydrogen, —OH, or —OR⁶ wherein at least one R⁵ group is —OR⁶;
wherein each R⁶ is independently: 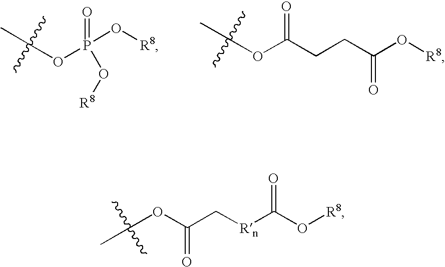
or a co-antioxidant; wherein R⁸ is hydrogen, alkyl, aryl, benzyl, Group IA metal, or a co-antioxidant; wherein R′ is CH₂; and where n is 1 to 9.

53. The method of claim 40, wherein one or more carotenoid derivatives or analogs have the structure: 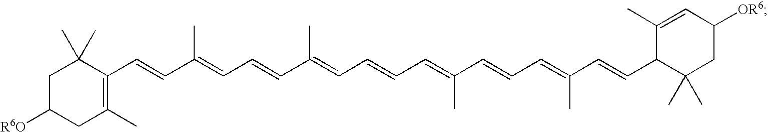
wherein each —OR⁶ is independently: 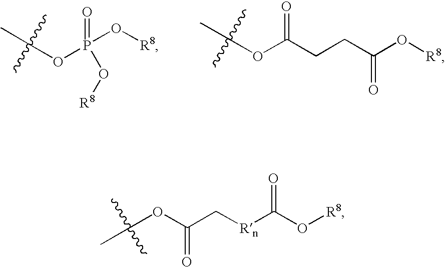
or a co-antioxidant; wherein R⁸ is hydrogen, alkyl, aryl, benzyl, Group IA metal, or a co-antioxidant; wherein R′ is CH₂; and where n is 1 to 9.

54. The method of claim 40, wherein one or more carotenoid derivatives or analogs have the structure: 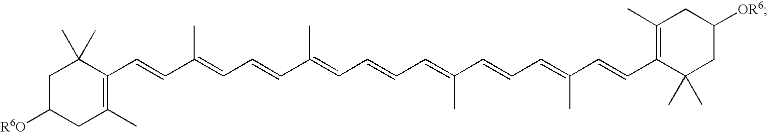
wherein each —OR⁶ is independently: 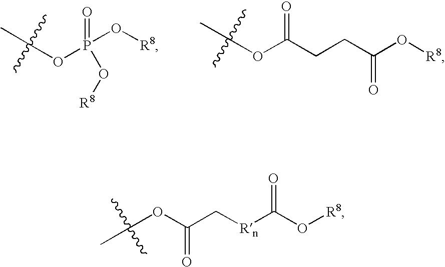
or a co-antioxidant; wherein R⁸ is hydrogen, alkyl, aryl, benzyl, Group IA metal, or a co-antioxidant; wherein R′ is CH₂; and where n is 1 to 9.

55. The method of claim 40, wherein the composition comprises two or more carotenoid derivatives or analogs having the structures: 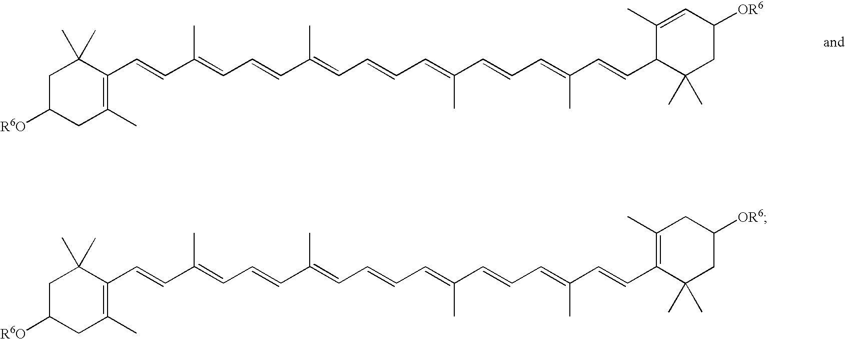
wherein each —OR⁶ is independently: 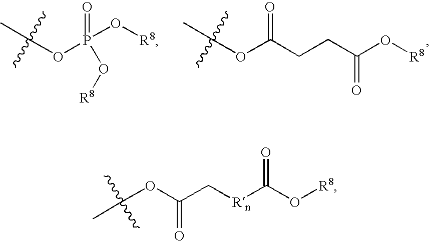
or a co-antioxidant; wherein R⁸ is hydrogen, alkyl, aryl, benzyl, Group IA metal, or a co-antioxidant; wherein R′ is CH₂; and where n is 1 to 9.

56. The method of claim 40, wherein each —OR⁶ independently comprises: 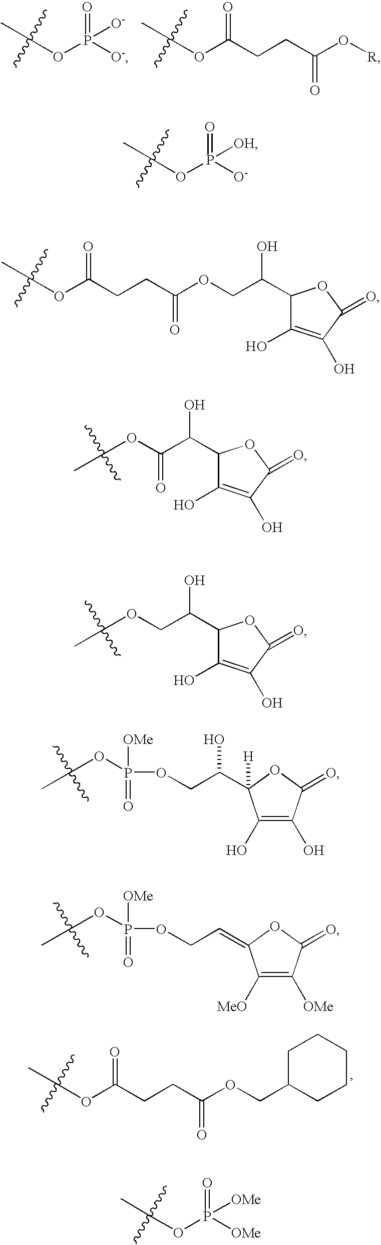
and wherein each R is independently H, alkyl, aryl, benzyl, Group IA metal, or co-antioxidant.

57. The method of claim 40, wherein each —OR⁶ independently comprises: 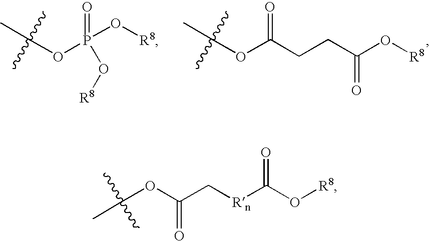
or a co-antioxidant; wherein R⁸ is hydrogen, alkyl, aryl, benzyl, Group IA metal, or a co-antioxidant; wherein R′ is CH₂; and where n is 1 to 9.

58-61. (canceled)

62. The method of claim 40, wherein one or more carotenoid derivatives or analogs have the structures: 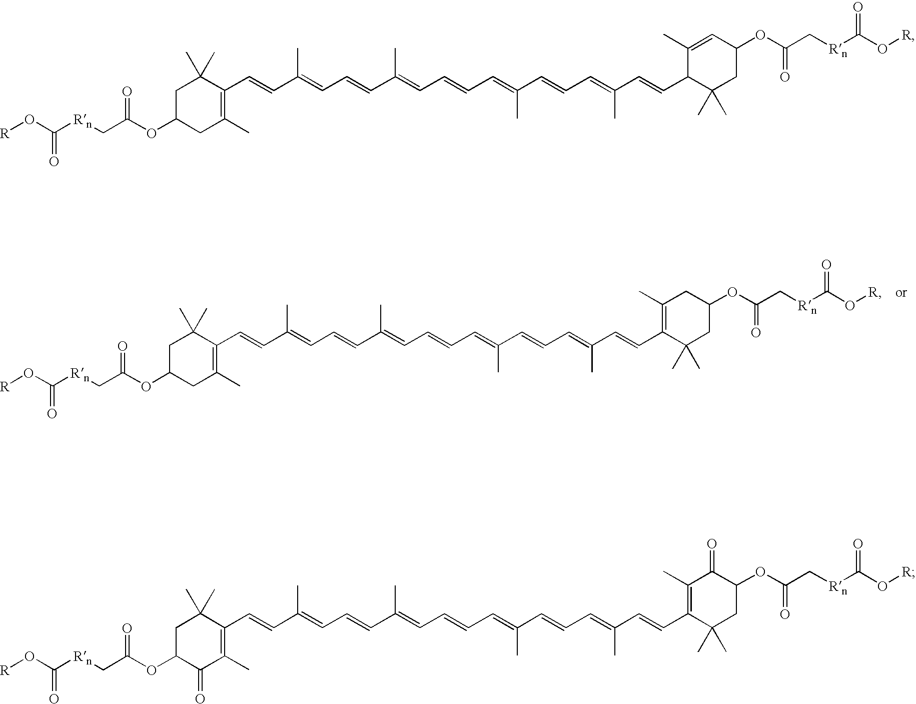
where each R is independently H, alkyl, aryl, benzyl, Group IA metal, or a co-antioxidant.

63. The method of claim 40, wherein one or more carotenoid derivatives or analogs have the structures: 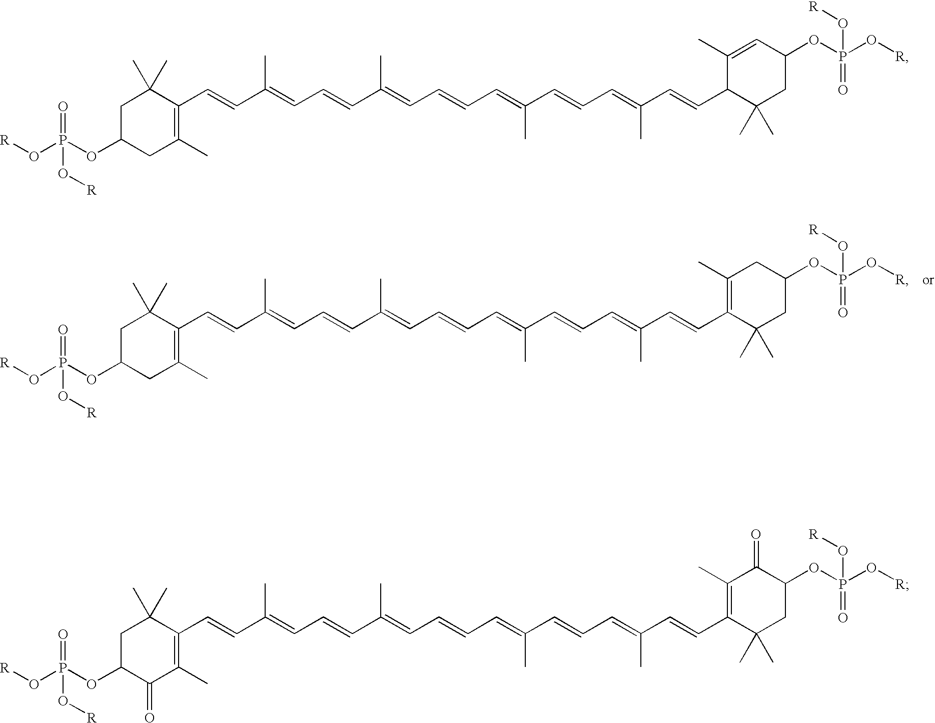
where each R is independently H, alkyl, aryl, benzyl, Group IA metal, or a co-antioxidant.

64. (canceled)

65. (canceled)

66. The method of claim 40, wherein one or more carotenoid derivatives or analogs have the structures: 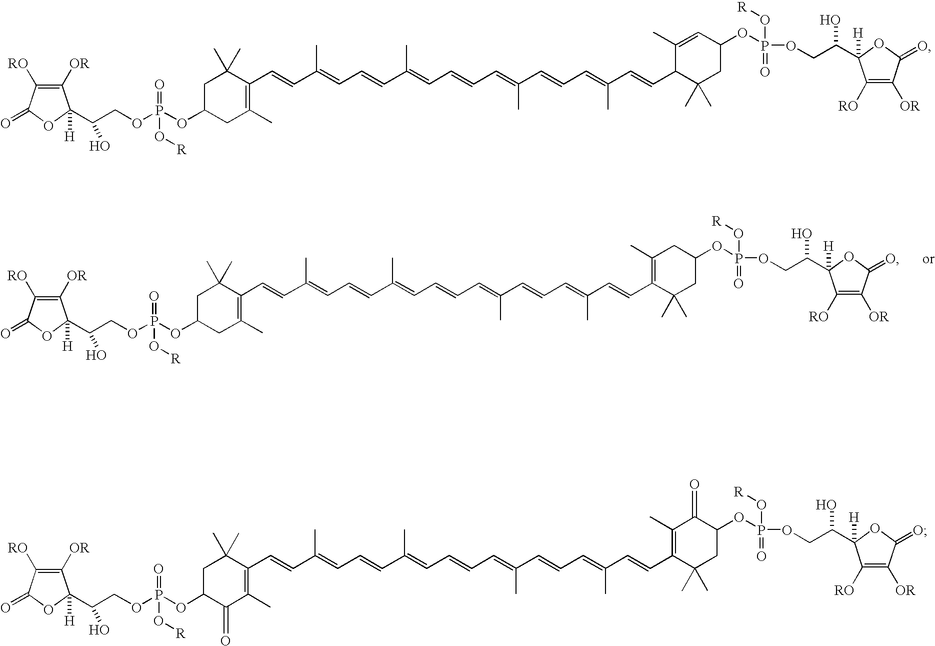
where each R is independently H, alkyl, aryl, benzyl, or a Group IA metal.

67. (canceled)

68. (canceled)

69. (canceled)

70. The method of claim 40, wherein the subject is human.

71. The method of claim 40, wherein the pharmaceutical composition is administered to the subject orally.

72. The method of claim 40, wherein the pharmaceutical composition is administered to the subject parenterally.

73. (canceled)

74. (canceled)

75. The method of claim 40, wherein the pharmaceutical composition is administered to the subject intravenously.

76-79. (canceled)

80. A method treating cancer in a subject comprising:
administering to a subject who would benefit from such treatment a therapeutically effective amount of a pharmaceutical composition comprising a carotenoid analog or derivative; and
administering to the subject a pharmaceutical composition comprising at least one anti-cancer agent;
wherein the carotenoid analog or derivative has the structure; 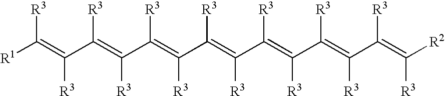
where each R³ is independently hydrogen or methyl, and where each R¹ and R² are independently: 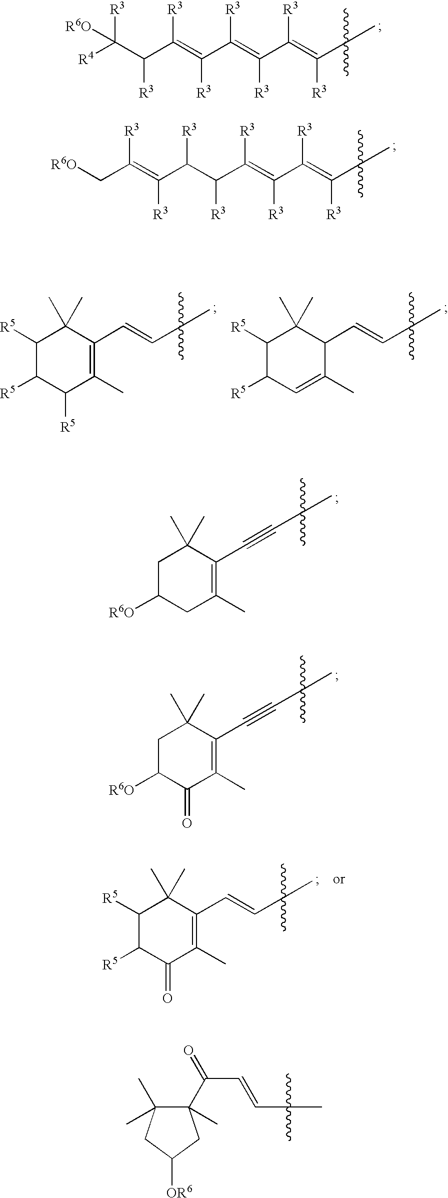
where R⁴ is hydrogen or methyl; where each R⁵ is independently hydrogen, —OH, or —OR⁶ wherein at least one R⁵ group is —OR⁶; wherein each R⁶ is independently: alkyl; aryl; -alkyl-N(R⁷)₂; -aryl-N(R⁷)₂; -alkyl-N⁺(R⁷)₃; -aryl-N⁺(R⁷)₃; -alkyl-CO₂R⁷; -aryl-CO₂R⁷; -alkyl-CO₂⁻; -aryl-CO₂⁻; —O—C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R8 is hydrogen, alkyl, aryl, benzyl or a co-antioxidant; where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

81. The method of claim 80, wherein the anticancer agent is a DNA-damaging agent, an agent that disrupts cell replication, a proteasome inhibitor, an NF-κB inhibitor, an IKK inhibitor, a topoisomerase I inhibitor, irinotecan, topotecan, camptothecin, doxorubicin, topoisomerase II inhibitor, etoposide, teniposide, daunorubicin, an alkylating agent, melphalan, chlorambucil, busulfan, thiotepa, ifosfamide, carmustine, lomustine, semustine, streptozocin, decarbazine, methotrexate, mitomycin C, and cyclophosphamide, a DNA intercalator, cisplatin, oxaliplatin, carboplatin, a free-radical generator, bleomycin, a nucleoside mimetics, 5-fluorouracil, capecitibine, gemcitabine, fludarabine, cytarabine, mercaptopurine, thioguanine, pentostatin, and hydroxyurea, paclitaxel, docetaxel, vincristine, vinblastin, thalidomide, CC-5013, CC-4047, a protein tyrosine kinase inhibitor, imatinib mesylate and gefitinib, an antibody that binds specifically to antigens expressed on the surface of cancer cells, trastuzumab, rituximab, cetuximab, bevacizumab, or analogs, derivatives or metabolite thereof.

82-85. (canceled)

86. A method of reducing the risk of occurrence of a proliferative disorder in a subject comprising administering to a subject who would benefit from chemopreventive therapy a prophylactically effective amount of pharmaceutical composition comprising a of a carotenoid analog or derivative having the structure; 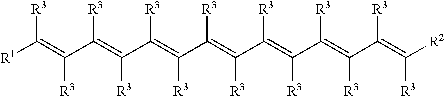
where each R³ is independently hydrogen or methyl, and where each R¹ and R² are independently: 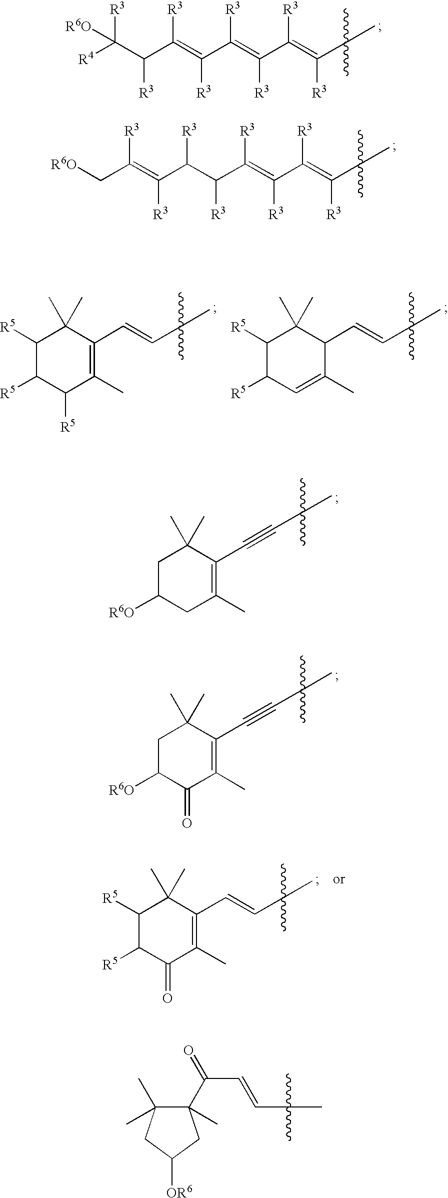
where R⁴ is hydrogen or methyl; where each R⁵ is independently hydrogen, —OH, or —OR⁶ wherein at least one R⁵ group is —OR⁶; wherein each R⁶ is independently: alkyl; aryl; -alkyl-N(R⁷)₂; -aryl-N(R⁷)₂; -alkyl-N⁺(R⁷)₃; -aryl-N⁺(R⁷)₃; -alkyl-CO₂R⁷; -aryl-CO₂R⁷; -alkyl-CO₂⁻; -aryl-CO₂⁻; —O—C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl or a co-antioxidant; where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

87. The method of claim 86, wherein pharmaceutical composition is adapted to be administered orally.

88. A pharmaceutical composition suitable for cancer chemotherapy comprising:
an amount of a carotenoid analog or derivative effective for cancer chemotherapy;
a delivery vehicle; and
one or more pharmacologically inert carriers,
wherein the carotenoid analog or derivative has the structure 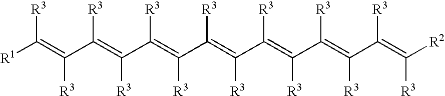
where each R³ is independently hydrogen or methyl, and where each R¹ and R² are independently: 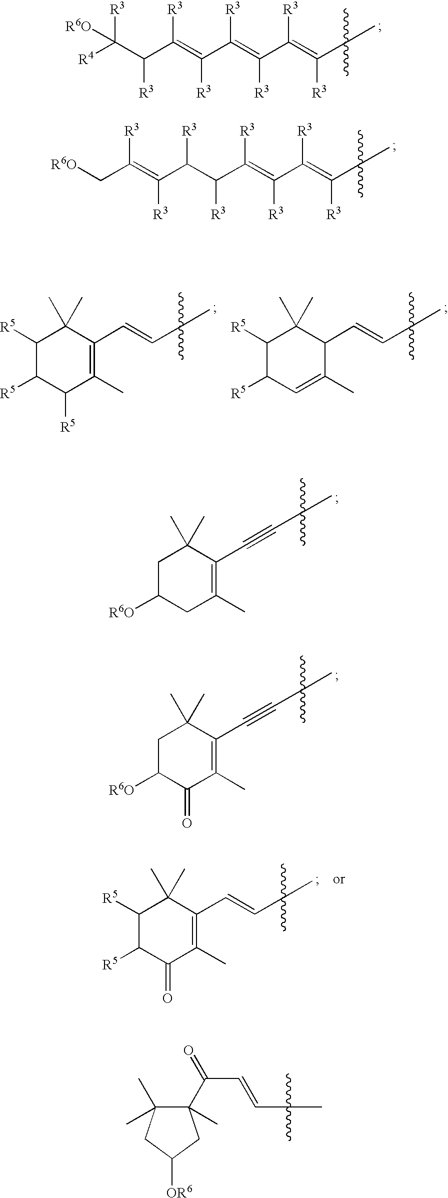
where R⁴ is hydrogen or methyl; where each R⁵ is independently hydrogen, —OH, or —OR⁶ wherein at least one R⁵ group is —OR⁶; wherein each R⁶ is independently: alkyl; aryl; -alkyl-N(R⁷)₂; -aryl-N(R⁷)₂; -alkyl-N⁺(R⁷)₃; -aryl-N⁺(R⁷)₃; -alkyl-CO₂R⁷; -aryl-CO₂R⁷; -alkyl-CO₂⁻; -aryl-CO₂⁻; —O—C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷is hydrogen, alkyl, or aryl; wherein R⁸ is hydrogen, alkyl, aryl, benzyl or a co-antioxidant; where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

89. The pharmaceutical composition of claim 88, further comprising an effective amount of at least one anticancer or chemotherapy agent.

90. The pharmaceutical composition of claim 89, wherein the anticancer agent is a DNA-damaging agent, an agent that disrupts cell replication, a proteasome inhibitor, an NF-κB inhibitor, an IKK inhibitor, a topoisomerase I inhibitor, irinotecan, topotecan, camptothecin, doxorubicin, topoisomerase II inhibitor, etoposide, teniposide, daunorubicin, an alkylating agent, melphalan, chlorambucil, busulfan, thiotepa, ifosfamide, carmustine, lomustine, semustine, streptozocin, decarbazine, methotrexate, mitomycin C, and cyclophosphamide, a DNA intercalator, cisplatin, oxaliplatin, carboplatin, a free-radical generator, bleomycin, a nucleoside mimetics, 5-fluorouracil, capecitibine, gemcitabine, fludarabine, cytarabine, mercaptopurine, thioguanine, pentostatin, and hydroxyurea, paclitaxel, docetaxel, vincristine, vinblastin, thalidomide, CC-5013, CC-4047, a protein tyrosine kinase inhibitor, imatinib mesylate and gefitinib, an antibody that binds specifically to antigens expressed on the surface of cancer cells, trastuzumab, rituximab, cetuximab, bevacizumab, or analogs, derivatives or metabolite thereof.
